(19) United States
(12) Patent Application Publication
(10) Pub. No.: US 2023/0340048 A1
(43) Pub. Date: Oct. 26, 2023

(54) TRANSFORMING GROWTH FACTOR BETA (TGFBETA) BINDING AGENTS AND USES THEREOF

(71) Applicant: Bristol-Myers Squibb TGF BETA Inc., Montreal (CA)

(72) Inventors: Maureen O’Connor, Beaconsfield (CA); Gilles Tremblay, La Prairie (CA); Jean-Francois Denis, Dollard-des-Ormeaux (CA); Vannakambadi K. Ganesh, Pearland, TX (US)

(21) Appl. No.: 18/009,506

(22) PCT Filed: Jun. 11, 2021
(86) PCT No.: PCT/CA2021/050795

§ 371 Date: Dec. 9, 2022

Related U.S. Application Data
(60) Provisional application No. 63/038,290, filed on Jun. 12, 2020.

(51) Int. Cl. C07K 14/495

(52) U.S. Cl. CPC C07K 14/495; A61K 38/00

(57) ABSTRACT

There are provided tetravalent TGFβ receptor-ectodomain based traps having a tailored isoform-specificity profile for neutralization of TGFβ ligands, and methods of use thereof in the treatment of diseases and conditions associated with TGFβ, particularly TGFβ1 and TGFβ3. In particular, there are provided TGFβ binding agents designed to tailor TGFβ isoform specificity, in order to maximize therapeutic efficacy in specific disease indications while minimizing adverse effects. The TGFβ binding agents comprise two polypeptides assembled via a multimerization domain, each polypeptide having two TGFβII receptor (TGFβR) ligand-binding domains linked as a doublet, wherein the linkers are selected to tailor isoform specificity.

### CROSS-REFERENCE TO RELATED APPLICATIONS

This application claims the benefit of U.S. Provisional Application No. 63/038,290, filed Jun. 12, 2020, the content of which is incorporated by reference herein in its entirety.

### REFERENCE TO SEQUENCE LISTING SUBMITTED ELECTRONICALLY

This application incorporates by reference in its entirety the Computer Readable Form (CRF) of a Sequence Listing submitted herewith. The Sequence Listing text file submitted herewith, entitled “14247-632-228_SEQ_LISTING.txt”, was created on Jun. 7, 2021, and is 247,915 bytes in size.

### FIELD

The present disclosure relates to TGFβ binding agents comprising TGFβ receptor ectodomain (TGFβR-ECD)-derived fusion molecules and uses thereof for binding and neutralizing TGFβ ligands, particularly for the treatment of diseases or conditions associated with TGFβ.

### BACKGROUND

Transforming growth factor beta (TGFβ) is part of a superfamily of over 30 ligands that regulate several physiological processes, including cell proliferation, migration and differentiation. Perturbation of their levels and/or signaling gives rise to significant pathological effects. TGFβ has been implicated in the pathogenesis of multiple human disorders (Akhurst, R.J. and Hata, A., 2012; Akhurst, R.J., 2017). For instance, TGFβ and activin ligands play critical pathogenic roles in many diseases including fibrosis and cancer. Examples of TGFβ-associated disorders include hematologic malignancies, solid tumors, bone marrow failure states, and a wide variety of disorders characterized by uncontrolled fibrosis such as pulmonary, liver, renal and vascular fibrosis, pulmonary arterial hypertension, and systemic sclerosis (SSc; also called scleroderma) (Nanthakumar, D.B. et al., 2015; Meng, X.-M. et al., 2016).

Persistent activation of TGFβ signaling plays a central role in the pathogenesis of fibrosis (Varga, J. and Whitfield, M.L., 2009). TGFβ canonical signaling stimulates the transition of fibroblasts to myofibroblasts (Desmouliere, A. et al., 1993; Midgley, A.C. et al., 2013) and plays a critical role in the production and deposition of collagen and other components of the extracellular matrix (ECM) (Prud’homme, G.J., 2007) as well as the induction of other mediators involved in fibrosis (Todd, N.W. et al., 2015). In patients with fibrotic disease such as scleroderma and idiopathic pulmonary fibrosis (IPF), TGFβ increases collagen deposition in the skin and/or lung, and stimulates fibroblast activation into myofibroblasts in the skin (Prud’homme, G.J., 2007; Lafyatis, R., 2014; Kissin, E.Y. et al., 2006). In addition, non-canonical TGFβ pathways also contribute to the maintenance of the fibrotic phenotype (Leask, A., 2008). Hence, the TGFβ signaling pathway has emerged as the most obvious target for therapeutic intervention in fibrosis (Varga, J. and Whitfield, M.L., 2009; Hunzelmann, N. and Krieg, T., 2010; Varga, J. and Pasche, B., 2008).

TGFβ is also considered as a critical regulator of tumor progression and is overexpressed by most tumor types. It favors tumorigenesis in part by inducing an epithelial-mesenchymal transition (EMT) in epithelial tumor cells, leading to aggressive metastasis. TGFβ also promotes tumorigenesis by acting as a powerful suppressor of the immune response in the tumor microenvironment. In fact, TGFβ is recognized to be one of the most potent immunosuppressive factors present in the tumor microenvironment. TGFβ interferes with the differentiation, proliferation and survival of many immune cell types, including dendritic cells, macrophages, NK cells, neutrophils, B-cells and T-cells; thus, it modulates both innate and adaptive immunity. The importance of TGFβ in the tumor microenvironment is highlighted by evidence showing that, in several tumor types including melanoma, lung, pancreatic, colorectal, hepatic and breast, elevated levels of TGFβ ligand are correlated with disease progression and recurrence, metastasis, and mortality. Hence, significant efforts have been invested in devising anti-tumor therapeutic approaches that involve TGFβ inhibition. These approaches include the use of polypeptide fusions based on a TGFβ receptor ectodomain that binds or “traps” the TGFβ ligand (see for example, WO01/83525; WO2005/028517; WO2008/113185; WO2008/157367; WO2010/0031168; WO2010/099219; WO2012/071649; WO2012/142515; WO2013/000234; WO2018/158727; US5693607; US2005/0203022; US2007/0244042; US8318135; US8658135; US8815247; US2015/0225483; and US2015/0056199).

One approach to developing therapeutic agents that inhibit TGFβ function has been to use antibodies or soluble decoy receptors (also termed receptor ectodomain (ECD)-based ligand traps) to bind and sequester ligand, thereby blocking access of ligand to its cell surface receptors. In general, receptor ECD-based traps are a class of therapeutic agents that are able to sequester selectively ligands, and that can be optimized using protein-engineering approaches.

Previously, it was shown that single-chain, bivalent TGFβ traps having two TGFβ receptor Type II (TGFβRII) ectodomains linked as a doublet can neutralize members of the TGFβ superfamily of ligands (WO2008/113185, WO2010031168). In those cases, bivalency was achieved by covalently linking two TGFβRII ectodomains using the intrinsically disordered regions (IDR) that flank the structured, ligand-binding domain of the TGFβRII ectodomain. It was further shown that potency increases when such bivalent doublets are joined in tandem to a multimerization domain such as an Fc component at the N- or C-terminus (WO2017/037634, WO2018/158727).

To date, most therapeutic approaches to neutralizing TGFβ have focused on the TGFβ1 isoform, especially in immune-oncology. This is because TGFβ1 is the predominantly expressed isoform in the immune system (Li, M.O. et al., 2006) as well as in many types of human tumors (Martin, C.J. et al., 2020). Although the intended target has usually been the TGFβ1 isoform, most therapeutic agents under development generally inhibit other TGFβ isoforms with varying potencies. For example, fresolimumab is a monoclonal antibody that is a pan-inhibitor of all three TGFβ isoforms. Although it neutralizes all isoforms, it inhibits the TGFβ1 isoform ~ 7-fold more potently than the TGFβ3 isoform, and ~14-fold more potently than the TGFβ2 isoform (Grutter, C. et al., 2008). This monoclonal antibody has been tested in clinical trials in cancer patients (Morris, J.C. et al., 2014; Lacouture, M.E. and Morris, J.C., 2015) and in patients with glomerulosclerosis (Vincenti, F. et al., 2017).

The TGFβ2 isoform has been implicated in cardiac homeostasis (Roberts, A.B. et al., 1992; Herbertz, S. et al., 2015), control of tumor dormancy (Bragado, P. et al., 2013), and the positive regulation of hematopoiesis (Langer, J.C. et al., 2004), suggesting that this isoform should be spared from neutralization since it plays beneficial roles.

Therefore, it would be useful to provide TGFβRII-ECD-based traps having tailored isoform specificity in order to maximize therapeutic efficacy in particular disease indications, while minimizing adverse effects. In particular, it may be useful to provide traps that neutralize TGFβ3 with potencies similar to that of TGFβ1.

### SUMMARY

There are provided herein tetravalent TGFβ receptor-ectodomain based traps having a tailored isoform-specificity profile for neutralization of TGFβ ligands, and methods of use thereof in the treatment of diseases and conditions associated with TGFβ. Tetravalent TGFβ binding agents provided herein comprise two polypeptides assembled via a multimerization domain, each polypeptide having two TGFβII receptor (TGFβR) ligand-binding domains linked as a doublet. TGFβ binding agents provided herein have been designed to tailor TGFβ isoform specificity, in order to maximize therapeutic efficacy in specific disease indications while minimizing adverse effects.

The present technology is based, at least in part, on the inventors’ realization that a TGFβ ligand trap with an isoform specificity that is differentiated from other known agents in development can be advantageous for the treatment of certain TGFβ-associated diseases and conditions. Recent reports have indicated an important role for the TGFβ3 isoform in certain TGFβ-associated conditions, such as fibrosis. For example, a recent report identified TGFβ3 as a key therapeutic target in kidney fibrosis by demonstrating that the specific downregulation of TGFβ3 by miR-29 counteracts renal fibrosis (Wang, H. et al., 2019). An important role for the TGFβ3 isoform in immunity was also suggested by recent reports on the production of TGFβ3 by immune cells (Komai, I.D. and Okamura, T., 2018). With respect to SSc, a genome-wide association study in African American patients identified TGFβ3 as a novel SSc susceptibility gene (Gourh, P. et al., 2017).

With respect to the TGFβ2, the implication of this isoform in cardiac homeostasis (Roberts, A.B. et al., 1992; Herbertz, S. et al., 2015), control of tumor dormancy (Bragado, P. et al., 2013), and the positive regulation of hematopoiesis (Langer, J.C. et al., 2004), has suggested that it may be desirable to avoid neutralization of this isoform.

Taken together, such findings suggest that neutralizing TGFβ1 and TGFβ3 to a similar extent may be beneficial for treatment of certain disorders, particularly those in which TGFβ3 is implicated. Achieving approximately equal inhibition of TGFβ1 and TGFβ3 may be useful, in some cases, to ensure both TGFβ1 and TGFβ3 can be neutralized effectively, which may prevent compensatory mechanisms that could occur when one of these isoforms is neutralized preferentially, and/or which may maximize efficacy. It is also desirable to inhibit TGFβ1 and TGFβ3 similarly without neutralizing TGFβ2 signaling, since it may be beneficial to avoid neutralization of this isoform.

In a broad aspect, there are provided herein novel polypeptide constructs useful for inhibiting an effect of a Transforming Growth Factor Beta (TGFβ) isoform. Polypeptides in accordance with the present disclosure comprise a TGFβ-binding region and a multimerization domain, wherein the N-terminus of the multimerization domain is joined to the C-terminus of the TGFβ-binding region. The TGFβ-binding region comprises two TGFβ receptor ligand-binding domains (TGFβR-LBDs) joined together by a first linker and joined to the multimerization domain by a second linker. In another broad aspect, there are provided TGFβ binding agents comprising two such polypeptide chains assembled via the multimerization domains, thereby forming a tetravalent molecule having a particular inhibition specificity for TGFβ ligands (TGFβ1, TGFβ2 and TGFβ3).

Without wishing to be limited by theory, the present invention is based, at least in part, on the finding that modifying one or more of the linkers in such a TGFβ ligand trap (e.g., the linker joining two TGFβR-LBDs together and/or the linker joining the TGFβR-LBDs to the multimerization domain) differentially affects inhibition potency of the binding agent for different TGFβ isoforms. It is shown herein that, in some cases, modifying one or both linker(s) can lower or equalize the TGFβ3:TGFβ1 IC₅₀ ratio (indicating similar or equalized inhibition potency for both isoforms), without increasing undesired inhibition of TGFβ2, and without significantly reducing the overall potency (e.g., IC₅₀ remains in the low picomolar range).

TGFβ binding agents provided herein generally comprise a first polypeptide and a second polypeptide that are associated together via the multimerization domain, each polypeptide comprising, in an N- to C- terminal orientation: an N-terminal region; a first TGFβ receptor ligand-binding domain ((TGFβR-LBD); a first linker; a second TGFβR-LBD; a second linker; and a multimerization domain. An embodiment of a TGFβ binding agent is shown schematically in FIG. 1 (which shows an embodiment where the TGFβ binding agent is a homodimer, i.e., the first and second polypeptides are the same). The first polypeptide and the second polypeptide may be bound to each other through their respective multimerization domains, e.g., by disulfide bonds (cysteine bridges), coiled coil interactions, and the like.

TGFβ binding agents of the present technology have been designed to lower or equalize the TGFβ3:TGFβ1 IC₅₀ ratio. That is, they have been designed to display reduced preferential inhibition of TGFβ1 compared to other known TGFβ traps. Accordingly, TGFβ binding agents provided herein are characterized by their specificity profile for isoform inhibition: specifically, the relative inhibition potency for the TGFβ1 and TGFβ3 isoforms (expressed herein as TGFβ3:TGFβ1 IC₅₀ ratio) is no more than about 2.5:1, and the activities of both TGFβ3 and TGFβ1 isoforms are inhibited at much greater potency than that of the TGFβ2 isoform (e.g., in the picomolar range for TGFβ3 and TGFβ1, and nanomolar for TGFβ2).

Additionally, in some embodiments the polypeptides and TGFβ binding agents of the present technology may provide certain advantages, in addition to tailored isoform specificity. For example and without limitation, the polypeptides and TGFβ binding agents may provide improved manufacturability, due for example to reduced glycosylation, increased homogeneity, ease of expression, and the like. Thus in some embodiments, polypeptides and TGFβ binding agents of the present technology provide one or more of the following advantages, relative to previous TGFβ binding agents: improved therapeutic effect for specific disease indications, for example TGFβ3-mediated conditions; reduced glycosylation; increased homogeneity; improved manufacturability; and increased production.

In some embodiments of polypeptides and TGFβ binding agents of the present technology, the first linker and the second linker are designed so as to provide the desired relative isoform-specificity of inhibition. For example, in some embodiments the lengths of the first linker and the second linker are selected such that the TGFβ3:TGFβ1 IC₅₀ ratio is no more than about 2.5:1, and both TGFβ3 and TGFβ1 isoform activity are inhibited at much greater potency than TGFβ2 isoform activity (e.g., in the picomolar range for TGFβ3 and TGFβ1, and nanomolar for TGFβ2).

In some embodiments of polypeptides and TGFβ binding agents of the present technology, the first linker and the second linker are selected so that the TGFβ3:TGFβ1 IC₅₀ ratio is about 2.5:1 or less. In some embodiments, the TGFβ3:TGFβ1 IC₅₀ ratio is less than about 2.5:1, about 2.3:1 or less, about 2:1 or less, about 1.8:1 or less, about 1.5:1 or less, about 1.3:1 or less, about 1.1:1 or less, about 1:1 or less, about 0.8:1 or less, or about 0.5:1 or less. In some embodiments, the TGFβ3:TGFβ1 IC₅₀ ratio for the TGFβ binding agent is from about 1:1 to about 2:1, or is about 1.1:1, 1.2:1, 1.3:1, 1.4:1, 1.5:1, 1.6:1, 1.7:1, 1.8:1, or 1.9:1. In a particular embodiment, the TGFβ3:TGFβ1 IC₅₀ ratio for the TGFβ binding agent is from about 1:1 to about 1.5:1 or from about 1.4:1 to about 1.6:1, or about 1.4:1, 1.5:1, or 1.6:1. In some such embodiments, the TGFβ binding agent inhibits both TGFβ1 isoform activity and TGFβ3 isoform activity with at least 20-fold, 100-fold, 200-fold, 300-fold, 400-fold, 500-fold, 600-fold, 700-fold, 800-fold, 900-fold, or 1000-fold greater potency than TGFβ2 isoform activity.

In some embodiments, the first linker is 33 amino acids or shorter. In an embodiment, the first linker is from about 10 to 33 amino acids long. In embodiments, the first linker may be from about 15 to 33 amino acids long, or from about 18 to about 30 amino acids long. In an embodiment, the first linker is 16, 18, 30, or 32 amino acids long. In a particular embodiment, the first linker is 16 amino acids long. In another particular embodiment, the first linker is 18 amino acids long. In another particular embodiment, the first linker is 30 amino acids long. In another particular embodiment, the first linker is 32 amino acids long.

In some embodiments, the second linker is 10 amino acids or longer. In an embodiment, the second linker is from about 10 to about 35 amino acids long. In embodiments, the second linker may be from about 10 to about 34 or from about 15 to about 34 amino acids long. In an embodiment, the second linker is 16, 30, 32, or 34 amino acids long. In a particular embodiment, the second linker is 30 amino acids long. In another particular embodiment, the second linker is 16 amino acids long. In another particular embodiment, the second linker is 32 amino acids long. In another particular embodiment, the second linker is 34 amino acids long.

In an embodiment, the first linker is 18 amino acids and the second linker is 16 amino acids. In another embodiment, the first linker is 18 amino acids and the second linker is 30 amino acids. In another embodiment, the first linker is 18 amino acids and the second linker is 10 amino acids. In another embodiment, the first linker is 18 amino acids and the second linker is 32 amino acids. In another embodiment, the first linker is 18 amino acids and the second linker is 34 amino acids. In another embodiment, the first linker is 16 amino acids and the second linker is 18 amino acids. In another embodiment, the first linker is 16 amino acids and the second linker is 16 amino acids. In another embodiment, the first linker is 16 amino acids and the second linker is 30 amino acids. In another embodiment, the first linker is 16 amino acids and the second linker is 32 amino acids. In another embodiment, the first linker is 26 amino acids and the second linker is 26 amino acids. In another embodiment, the first linker is 32 amino acids and the second linker is 32 amino acids. In another embodiment, the first linker is 32 amino acids and the second linker is 34 amino acids. It should be understood that many other permutations are possible, as long as the desired isoform-specificity of inhibition is achieved.

In some embodiments, one or more of the first linker and the second linker comprises or consists of an IDR linker, an IDR linker variant, a hybrid linker, a hybrid linker variant, a truncated linker, a truncated linker variant or an elongated linker disclosed herein. For example, one or more of the first linker and the second linker may independently comprise or consist of the amino acid sequence set forth in any one of SEQ ID NOs: 4 or 8-26, or a sequence at least 80%, at least 85%, at least 90%, at least 95%, at least 96%, at least 97%, at least 98%, or at least 99% identical thereto. In some embodiments, the first linker comprises or consists of the amino acid sequence set forth in any one of SEQ ID NOs: 8, 9, 10, 11, 12, 13, 14, 16, 21, 22, 23, and 26, or a sequence at least 90%, at least 95%, at least 96%, at least 97%, at least 98%, or at least 99% identical thereto. In some embodiments, the second linker comprises or consists of the amino acid sequence set forth in any one of SEQ ID NOs: 4, 9, 11, 15, 17, 18, 19, 20, 22, 23, 24, 25, and 26, or a sequence at least 90%, at least 95%, at least 96%, at least 97%, at least 98%, or at least 99% identical thereto.

In a representative embodiment, the first linker comprises or consists of the amino acid sequence set forth in SEQ ID NO: 12 and/or the second linker comprises or consists of the amino acids sequence set forth in SEQ ID NO: 11. In another representative embodiment, the first linker comprises or consists of the amino acid sequence set forth in SEQ ID NO: 8 and/or the second linker comprises or consists of the amino acids sequence set forth in SEQ ID NO: 9. It should be understood that other embodiments using combinations of linkers provided herein are encompassed, as long as the desired isoform-specificity of inhibition is achieved.

In some embodiments of polypeptides and TGFβ binding agents of the present technology, the N-terminal region comprises or consists of an IDR linker, an IDR linker variant, a hybrid linker, a hybrid linker variant, a truncated linker, a truncated linker variant or an elongated linker. For example, the N-terminal region may comprise or consist of the amino acid sequence set forth in SEQ ID NO: 3, or a sequence at least 90%, at least 95%, at least 96%, at least 97%, at least 98%, or at least 99% identical thereto.

In some embodiments of polypeptides and TGFβ binding agents of the present technology, the first TGFβR-LBD and/or the second TGFβR-LBD comprises or consists of the amino acid sequence set forth in SEQ ID NO: 2, or a sequence at least 90%, at least 95%, at least 96%, at least 97%, at least 98%, or at least 99% identical thereto. In some embodiments, the first TGFβR-LBD and the second TGFβR-LBD are the same or substantially the same. In other embodiments, the first TGFβR-LBD and the second TGFβR-LBD may have different amino acid sequences.

In some embodiments of polypeptides and TGFβ binding agents of the present technology, the multimerization domain allows dimerization of two polypeptides in accordance with the present disclosure, in a non-covalent manner, e.g., by coiled-coil interactions, and the like.

In other embodiments, the multimerization domain allows dimerization of two polypeptides in accordance with the present disclosure in a covalent manner, e.g., by disulfide bridging, and the like.

In some embodiments, the multimerization domain comprises one or more constant region of an antibody, e.g., the second constant domain (C_{H}2) and/or the third constant domain (C_{H}3) of an antibody heavy chain, or an Fc region of an antibody heavy chain. The antibody may be, for example and without limitation, an IgG antibody such as an IgG1, IgG2, IgG3 or IgG4 antibody. In particular embodiments, the antibody is a human antibody, e.g., the multimerization domain comprises a constant region of the heavy chain of a human IgG1, IgG2, IgG3 or IgG4. In some embodiments, the multimerization domain has at least 80%, at least 85%, at least 90%, at least 95%, at least 96%, at least 97%, at least 98%, or at least 99% sequence identity with a human IgG1, IgG2, IgG3 or IgG4 constant region. In a particular embodiment, the multimerization domain comprises or consists of an Fc region of a human IgG1 antibody. In another particular embodiment, the multimerization domain comprises or consists of an Fc region of a human IgG4 antibody.

In some embodiments, the multimerization domain comprises one or more cysteine residue for crosslinking of a first polypeptide construct with a second polypeptide construct. For example, the multimerization domain may include at least two cysteine residues for forming a disulfide bridge between two polypeptide constructs, thereby forming a dimer.

In some embodiments, the multimerization domain is engineered to reduce aggregation or to modulate stability of a dimer or multimer of the polypeptide construct. For example, an Fc region may contain one or more amino acid substitution that reduces aggregation and/or increases stability of the TGFβ binding agent compared to naturally occurring Fc sequences. In some embodiments, the multimerization domain is selected to provide one or more effector function such as antibody dependent cellular cytotoxicity (ADCC), complement activation (complement dependent cytotoxicity or CDC), opsonization, and the like.

In some embodiments, the multimerization domain comprises or consists of the amino acid sequence set forth in any one of SEQ ID NOs: 49-80 or a sequence at least 80%, at least 85%, at least 90%, at least 95%, at least 96%, at least 97%, at least 98%, or at least 99% identical thereto. In a particular embodiment, the multimerization domain comprises or consists of the amino acid sequence set forth in SEQ ID NO:49, or a sequence at least 80%, at least 85%, at least 90%, at least 95%, at least 96%, at least 97%, at least 98%, or at least 99% identical thereto. In another particular embodiment, the multimerization domain comprises or consists of the amino acid sequence set forth in SEQ ID NO:50, or a sequence at least 80%, at least 85%, at least 90%, at least 95%, at least 96%, at least 97%, at least 98%, or at least 99% identical thereto.

In some embodiments of polypeptides and TGFβ binding agents of the present technology, the TGFβ binding region (comprising the N-terminal domain, the two LBDs, and the two linkers) comprises or consists of the sequence set forth in any one of SEQ ID NOs: 27-48, or a sequence at least 80%, at least 85%, at least 90%, at least 95%, at least 96%, at least 97%, at least 98%, or at least 99% identical thereto. In a particular embodiment, the TGFβ-binding region comprises or consists of the amino acid sequence set forth in SEQ ID NO: 27. In another particular embodiment, the TGFβ-binding region comprises or consists of the amino acid sequence set forth in SEQ ID NO: 29. In another particular embodiment, the TGFβ-binding region comprises or consists of the amino acid sequence set forth in SEQ ID NO: 32. In another particular embodiment, the TGFβ-binding region comprises or consists of the amino acid sequence set forth in SEQ ID NO: 41.

In another particular embodiment, the TGFβ-binding region comprises or consists of the amino acid sequence set forth in SEQ ID NO: 40.

In some embodiments of polypeptides and TGFβ binding agents of the present technology, the polypeptide construct comprises or consists of the amino acid sequence set forth in any one of SEQ ID NOs: 81 to 103 and 105, or a sequence at least 80%, at least 85%, at least 90%, at least 95%, at least 96%, at least 97%, at least 98%, or at least 99% identical thereto. In a particular embodiment, the polypeptide construct comprises or consists of the amino acid sequence set forth in SEQ ID NO: 81. In another particular embodiment, the polypeptide construct comprises or consists of the amino acid sequence set forth in SEQ ID NO: 84. In another particular embodiment, the polypeptide construct comprises or consists of the amino acid sequence set forth in SEQ ID NO: 87. In another particular embodiment, the polypeptide construct comprises or consists of the amino acid sequence set forth in SEQ ID NO: 96.

In another particular embodiment, the polypeptide construct comprises or consists of the amino acid sequence set forth in SEQ ID NO: 95, or a sequence at least 80%, at least 85%, at least 90%, at least 95%, at least 96%, at least 97%, at least 98%, or at least 99% identical thereto. In another particular embodiment, the polypeptide construct comprises or consists of the amino acid sequence set forth in SEQ ID NO: 95.

In some embodiments, the polypeptide construct provided herein is a polypeptide construct comprising N-terminus to C-terminus: (i) an amino acid sequence consisting of the amino acid sequence of SEQ ID NO:40; and (ii) an Fc region of human IgG1.

In some embodiments of the present technology, a TGFβ binding agent is heterodimeric, that is, the first and the second polypeptide are different. In such embodiments, the first and the second polypeptide may differ by one or more region or domain, e.g., by the sequence of the first linker, the second linker, the LBD, the multimerization domain, etc., as well as combinations thereof. Thus each of the following may independently be the same or different in the two polypeptides: the N-terminal region; the first linker; the second linker; the first LBD; the second LBD; and the multimerization domain. Many combinations are possible, as long as the desired isoform-specificity of inhibition is provided.

In some embodiments of the TGFβ binding agent, the first polypeptide construct and the second polypeptide construct comprises or consists of the sequence set forth in SEQ ID NO:95, or a sequence at least 80%, at least 85%, at least 90%, at least 95%, at least 96%, at least 97%, at least 98%, or at least 99% identical thereto. In some embodiments of the TGFβ binding agent, the first polypeptide construct and the second polypeptide construct comprises or consists of the sequence set forth in SEQ ID NO:95.

In some embodiments of the TGFβ binding agent, the different TGFβ-binding regions comprise or consist of the amino acid sequence set forth in SEQ ID NO:95, or a sequence at least 80%, at least 85%, at least 90%, at least 95%, at least 96%, at least 97%, at least 98%, or at least 99% identical thereto.

In some embodiments of the TGFβ binding agent, the TGFβ binding agent comprises:
a first polypeptide construct comprising from N-terminus to C-terminus: (i) an amino acid sequence consisting of the amino acid sequence of SEQ ID NO:40; and (ii) a first Fc region of human IgG1, and
a second polypeptide construct comprising from N-terminus to C-terminus: (i) an amino acid sequence consisting of the amino acid sequence of SEQ ID NO:40; and (ii) a second Fc region of human IgG1;
wherein the first polypeptide construct and the second polypeptide construct are linked together through the first and second Fc region of human IgG1.

In some embodiments, the inhibitory potency of the TGFβ binding agent for both TGFβ1 isoform activity and TGFβ3 isoform activity is greater than for TGFβ2 isoform activity; and wherein the relative inhibitory potency of the TGFβ binding agent for TGFβ3 isoform activity compared to TGFβ1 isoform activity (IC₅₀ ratio for TGFβ3 TGFβ1) is about 2.5:1 or less.

In some embodiments, the TGFβ binding agent provided herein is a homodimer of the polypeptide construct provided herein.

In alternate embodiments, a TGFβ binding agent is homodimeric, that is, the first and the second polypeptide are the same or substantially the same.

In some embodiments, the polypeptide or the TGFβ binding agent may be conjugated with a targeting agent, a therapeutic moiety, a detectable moiety and/or a diagnostic moiety.

In another broad aspect, there are provided nucleic acids encoding the polypeptides and TGFβ binding agents of the present technology. Vectors and plasmids comprising such nucleic acids and/or for expression of the polypeptides and TGFβ binding agents are also provided. For example, in an embodiment there is provided a nucleic acid having the sequence set forth in any one of SEQ ID NOs: 106-109, and vectors and plasmids comprising these nucleic acids. In another embodiment, there is provided a nucleic acid having at least 80% sequence identity, at least 85% sequence identity, at least 90% sequence identity, at least 95% sequence identity, at least 98% sequence identity, or at least 99% sequence identity to SEQ ID NOs: 106-109, or capable of hybridizing thereto under conditions of high stringency. Cells expressing the polypeptides and TGFβ binding agents of the present technology are also provided.

In further aspects, there are provided methods of manufacturing the polypeptides and TGFβ binding agents of the present technology, comprising expressing one or more polypeptide provided herein in a cell, followed by isolation and/or purification thereof. In some embodiments, polypeptide constructs and TGFβ binding agents are expressed in a form that is secretable by a cell, e.g., using a signal peptide at the N-terminus, allowing recovery of the polypeptide or TGFβ binding agent from the culture medium.

In another broad aspect, there are provided pharmaceutical compositions comprising the polypeptide construct or the TGFβ binding agent according to the present disclosure and a pharmaceutically acceptable carrier, diluent or excipient. In some embodiments, pharmaceutical compositions are formulated for administration by injection or infusion, e.g., for intravenous, subcutaneous, intraperitoneal, or intramuscular administration. In some embodiments, pharmaceutical compositions are provided in unit dosage form.

In yet another broad aspect, there are provided methods of preventing or treating a TGFβ-associated disease or condition, the methods comprising administering a therapeutically effective amount of the polypeptide, TGFβ binding agent or pharmaceutical composition of the present technology to a subject, such that the TGFβ-associated disease or condition is prevented or treated.

Examples of TGFβ-associated diseases or conditions that may be prevented or treated in accordance with the present disclosure include, for example and without limitation: fibrosis (e.g., fibrotic disease, fibrotic scarring, fibroproliferative disorders); cancer (e.g., malignancies, solid tumors, metastasis); and bone marrow failures (e.g., Shwachman-Bodian-Diamond syndrome, Fanconi anemia). In some embodiments, the polypeptide or TGFβ binding agent described herein is used for the treatment or prevention of fibrosis, including for example and without limitation, fibrotic disease of tissues and/or organs, and fibrotic scarring, e.g., pulmonary fibrosis (e.g., idiopathic pulmonary fibrosis), renal fibrosis, liver fibrosis (e.g., hepatic cirrhosis), systemic sclerosis, scleroderma, skin fibrosis, heart fibrosis, myelofibrosis, etc.

In some embodiments, there are provided methods of preventing or treating a disease or condition mediated by TGFβ1 and/or TGFβ3, the methods comprising administering a therapeutically effective amount of the polypeptide, TGFβ binding agent or pharmaceutical composition of the present technology to a subject, such that the disease or condition mediated by TGFβ1 and/or TGFβ3 is treated. In an embodiment, there is provided a method of preventing or treating a disease or condition mediated by TGFβ3 in a subject in need thereof, the method comprising administering a therapeutically effective amount of the polypeptide, TGFβ binding agent or pharmaceutical composition of the present technology to the subject, such that the disease or condition mediated by TGFβ3 is prevented or treated.

In some embodiments, there are provided methods of preventing or treating fibrosis in a subject in need thereof, the method comprising administering a therapeutically effective amount of the polypeptide, TGFβ binding agent or pharmaceutical composition of the present technology to the subject, such that fibrosis is prevented or treated.

In a further broad aspect, there are provided kits and packages for treating a TGFβ-associated disease or condition in a subject in need thereof, comprising a polypeptide, a TGFβ binding agent or a pharmaceutical composition in accordance with the present disclosure; optionally one or more additional component such as acids, bases, buffering agents, inorganic salts, solvents, antioxidants, preservatives, or metal chelators, and/or tools for administration thereof such as syringes, needles, and the like. Instructions for administration or use may also be included.

In yet another aspect, provided herein are methods of manufacturing of the polypeptide construct or the TGFβ binding agent provided herein, comprising culturing the host cell as provided herein under conditions suitable for protein expression; and harvesting the polypeptide construct or the TGFβ binding agent.

In yet another aspect, provided herein are polypeptide constructs or TGFβ binding agents produced by the manufacturing methods provided herein.

Further scope, applicability and advantages of the present disclosure will become apparent from the non-restrictive detailed description given hereinafter. It should be understood, however, that this detailed description, while indicating exemplary embodiments of the disclosure, is given by way of example only, with reference to the accompanying drawings.

### BRIEF DESCRIPTION OF THE DRAWINGS

For a better understanding of the technology and to show more clearly how it may be carried into effect, reference will now be made by way of example to the accompanying drawings, which illustrate aspects and features according to non-limiting embodiments of the present technology.

FIG. 1 shows a schematic structure of the domain organization of tetravalent TGFβ binding agents, in accordance with certain embodiments. The embodiment shown here is a homodimer of a first polypeptide (Left side) and a second polypeptide (Right side) linked by a disulfide bridge in the multimerization domain (shown as two lines). Ligand-binding domains (LBDs) are shown as circles, multimerization domains are shown as ovals, and N-terminal regions and linkers are shown as rectangles. In embodiments where the binding agent is a heterodimer, the first and second polypeptides are different in one or more region or portion (not shown).

FIG. 2A shows an overlay of the monomeric structures of TGFβ1 (blue) and TGFβ3 (green).

FIG. 2B shows an overlay of the TGFβ1 dimer (blue) and the TGFβ3 dimer (green). The corresponding monomers in the area are superposed to show the monomer difference in the dimer angle.

FIG. 2C shows a representative model of T22d35-Fc-IgG1-v1(CC) (SEQ ID NO: 6) bound to TGFβ ligand showing the second ligand binding domain, second linker, and multimerization domain (Fc) regions.

FIG. 3A shows polyacrylamide gel electrophoresis analysis under non-reducing conditions of representative TGFβ binding agents. After expression and purification, 2 µg of each protein was loaded on the gel, as indicated: Ctl: Control; p61: Protein 61; p96: Protein 96; p101: Protein 101; p107: Protein 107; p112: Protein 112.

FIG. 3B shows polyacrylamide gel electrophoresis analysis under reducing conditions of representative TGFβ binding agents. After expression and purification, 2 ug of each protein was loaded on the gel, as indicated: Ctl: Control; p61: Protein 61; p96: Protein 96; p101: Protein 101; p107: Protein 107; p112: Protein 112.

FIG. 4A shows representative results in the A549/IL-11 cell-based assay for inhibition of TGFβ1 for Proteins 61, 96, 101, 107, and 112, and Control, as indicated. The table lists the calculated IC₅₀ values calculated in Graphpad Prism. Error bars indicate standard error of the mean (SEM).

FIG. 4B shows representative results in the A549/IL-11 cell-based assay for inhibition of TGFβ3 for Proteins 61, 96, 101, 107, and 112, and Control, as indicated. The table lists the calculated IC₅₀ values calculated in Graphpad Prism. Error bars indicate standard error of the mean (SEM).

FIG. 5A shows polyacrylamide gel electrophoresis analysis under non-reducing conditions of the following representative TGFβ binding agents: p112, p111, p108, p105, p104, p101, p99, and p71. Error bars indicate standard error of the mean (SEM).

FIG. 5B shows polyacrylamide gel electrophoresis analysis under reducing conditions of the following representative TGFβ binding agents: p112, p111, p108, p105, p104, p101, p99, and p71. Error bars indicate standard error of the mean (SEM).

FIG. 6A shows representative results in the A549/IL-11 cell-based assay for inhibition of TGFβ1 for Proteins 113, 115, and 116, and Control, as indicated. The table lists the calculated IC₅₀ values calculated in Graphpad Prism. Error bars indicate standard error of the mean (SEM).

FIG. 6B shows representative results in the A549/IL-11 cell-based assay for inhibition of TGFβ3 for Proteins 113, 115, and 116, and Control, as indicated. The table lists the calculated IC₅₀ values calculated in Graphpad Prism. Error bars indicate standard error of the mean (SEM).

FIG. 7A shows representative results in the A549/IL-11 cell-based assay for inhibition of TGFβ1 for Proteins 101, 129, and 130, and Control, as indicated. The table lists the calculated IC₅₀ values calculated in Graphpad Prism. Error bars indicate standard error of the mean (SEM).

FIG. 7B shows representative results in the A549/IL-11 cell-based assay for inhibition of TGFβ3 for Proteins 101, 129, and 130, and Control, as indicated. The table lists the calculated IC₅₀ values calculated in Graphpad Prism. Error bars indicate standard error of the mean (SEM).

FIG. 8A shows representative results in the A549/IL-11 cell-based assay for inhibition of TGFβ1 for Proteins 101, 131, 132 and 133, and Control, as indicated. The table lists the calculated IC₅₀ values calculated in Graphpad Prism. Error bars indicate standard error of the mean (SEM).

FIG. 8B shows representative results in the A549/IL-11 cell-based assay for inhibition of TGFβ3 for Proteins 101, 131, 132 and 133, and Control, as indicated. The table lists the calculated IC₅₀ values calculated in Graphpad Prism. Error bars indicate standard error of the mean (SEM).

FIG. 9A shows representative results in the A549/IL-11 cell-based assay for inhibition of TGFβ1 for Proteins 96, 134, and 135, and Control, as indicated. The table lists the calculated IC₅₀ values calculated in Graphpad Prism. Error bars indicate standard error of the mean (SEM).

FIG. 9B shows representative results in the A549/IL-11 cell-based assay for inhibition of TGFβ3 for Proteins 96, 134, and 135, and Control, as indicated. The table lists the calculated IC₅₀ values calculated in Graphpad Prism. Error bars indicate standard error of the mean (SEM).

FIG. 10A shows representative results in the A549/IL-11 cell-based assay for inhibition of TGFβ1 for Proteins 101 and 128, and Control, as indicated. The table lists the calculated IC₅₀ values calculated in Graphpad Prism. Error bars indicate standard error of the mean (SEM).

FIG. 10B shows representative results in the A549/IL-11 cell-based assay for inhibition of TGFβ3 for Proteins 101 and 128, and Control, as indicated. The table lists the calculated IC₅₀ values calculated in Graphpad Prism. Error bars indicate standard error of the mean (SEM).

FIG. 11 shows representative results in the A549/IL-11 cell-based assay for inhibition of TGFβ2 for Proteins 61, 96 and 101, and Control, as indicated. The table lists the calculated IC₅₀ values calculated in Graphpad Prism. Error bars indicate standard error of the mean (SEM).

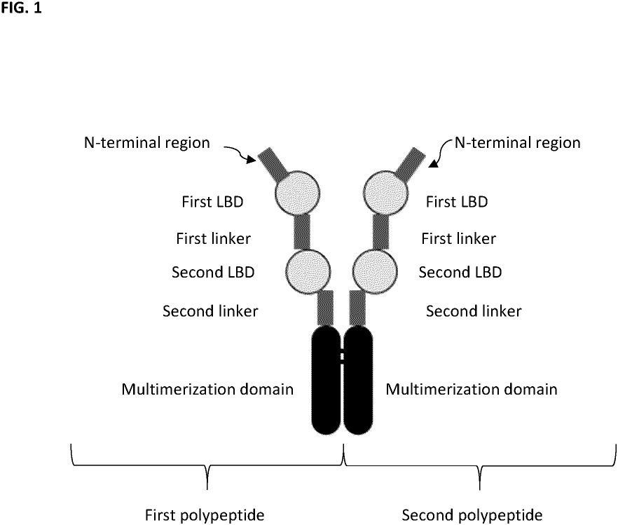

### DETAILED DESCRIPTION

The present technology is explained in greater detail below. This description is not intended to be a detailed catalog of all the different ways in which the technology may be implemented, or all the features that may be added to the instant technology. For examples, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure, which variations and additions do not depart from the present technology. Hence, the following description is intended to illustrate some particular embodiments of the technology, and not to exhaustively specify all permutations, combinations and variations thereof.

### Definitions

In order to provide a clear and consistent understanding of the terms used in the present specification, a number of definitions are provided below. Moreover, unless defined otherwise, all technical and scientific terms as used herein have the same meaning as commonly understood to one of ordinary skill in the art to which this invention pertains.

The use of the terms “a” and “an” and “the” when used in conjunction with the term “comprising” in the claims and/or the specification may mean “one”, but it is also consistent with the meaning of “one or more”, “at least one”, and “one or more than one”. Similarly, the term “another” may mean at least a second or more. These terms are to be construed to cover both the singular and the plural, unless otherwise indicated herein or clearly contradicted by context.

As used herein, the terms “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps. The term “consisting of” is to be construed as close-ended.

The term “about” is used to indicate that a value or quantity refers to the actual given value and also the approximation of such given value that would reasonably be inferred based on the ordinary skill in the art, including equivalents and approximations due to the experimental and/or measurement conditions for such given value. For example, the term “about” in the context of a given value or range refers to a value or range that is within 20%, preferably within 15%, more preferably within 10%, more preferably within 9%, more preferably within 8%, more preferably within 7%, more preferably within 6%, and more preferably within 5% of the given value or range.

The expression “and/or” where used herein is to be taken as specific disclosure of each of the specified features or components with or without the other. For example “A and/or B” is to be taken as specific disclosure of each of (i) A, (ii) B, and (iii) A and B, just as if each is set out individually herein. Unless specifically stated or obvious from context, as used herein the term “or” is understood to be inclusive and covers both “or” and “and”. For example, an embodiment of “a composition comprising A or B” would typically present an aspect with a composition comprising both A and B. “Or” should, however, be construed to exclude those aspects presented that cannot be combined without contradiction (e.g., a composition pH that is between 9 and 10 or between 7 and 8).

It is to be understood herein that terms such as “from 1 to 20” include any individual values comprised within and including 1 and 20. Therefore, the term “from 1 to 20” includes 1, 2, 3, 4, 5, 6, 7, 8, 9, 10, 11, 12, 13, 14, 15, 16, 17, 18, 19 and/or 20. Terms such as “from 1 to 20” also include any individual sub-ranges comprised within and including from 1 to 20. The term “from 1 to 20” therefore also includes sub-ranges such as “from 1 to 9”, “from 2 to 9”, “from 3 to 5”, from 5 to 9”, “from 5 to 20”, “from 8 to 20” etc. The same applies for similar expressions such as and not limited to “from 1 to 19”, “from 1 to 18”, “from 1 to 10”, “from 1 to 9”, “from 5 to 15”, etc.

It is to be understood herein that terms such as “from about 15 to about 35” include any individual values comprised within and including 15 and 35. Therefore, terms such as “from about 15 to about 35” include any number between and including 15 and 35 such as 15, 16, 17, 18, 19, 20, 21, 22, 23, 24, 25, 26, 27, 28, 29, 30, 31, 32, 33, 34 and/or 35. Terms such as “from about 15 to about 35” also include any individual sub-ranges comprised within and including from 15 to 35, “from about 16 to about 34”, “from about 16 to about 24”, from about 24 to about 34” and the like. The term “about” in the context of the number of amino acids means that the specified number of amino acids is specifically encompassed and allows a variation of +/- 2 in the number of amino acid residues. As such, the terms such as “from about 15 to about 35” also includes “from 13 to 37”, “from 13 to 35”, “from 17 to 37”, from 17 to 35”, etc. The same applies for similar expressions such as and not limited to “from about 16 to about 34”, “from about 16 to about 24”, from about 24 to about 34” and the like.

It is to be understood herein that terms such as “at least 80% identical” include any individual values comprised within and including from 80% to 100% and including 80%, 81%, 82%, 83%, 84%, 85%, 86%, 87%, 88%, 89%, 90%, 91%, 92%, 93%, 94%, 95%, 96%, 97%, 98%, 99% and 100%. The term “at least 80% identical” also includes any individual sub-ranges comprised within and including from 80% to 100%, such as for example, “from 85% to 99%”, “from 97% to 100%”, “from 90% to 100%”, etc. The same applies for similar expressions such as, and not limited to, expressions such as “at least 70% identical”, “at least 90% identical”, and the like.

As used herein, the term “inhibition potency” refers to effectiveness of a substance in inhibiting a specific biological or biochemical function such as, without limitation, binding between a protein receptor and its ligand, or activation of a cell receptor by its ligand. In some embodiments, potency of inhibition is determined by measuring the IC50 of an inhibitor for a particular ligand or substrate. In that case, relative inhibition potency for different inhibitors and/or ligands may be assessed by comparing IC₅₀ values. For example, relative inhibition potency of 3:1 means the ratio of IC₅₀ values is 3:1. The terms “inhibition potency”, “inhibitory potency”, “potency of inhibition” and “neutralization potency” are used interchangeably herein.

As used herein, the term “IC₅₀” refers to the half maximal inhibitory concentration (i.e., the concentration of a substance that is required for 50% inhibition in vitro). It is a measure of the potency or effectiveness of a substance in inhibiting a specific biological or biochemical function. IC₅₀ values are typically expressed as molar concentration. The IC₅₀ of an inhibitor can be determined by constructing a dose-response curve and examining the effect of different concentrations of inhibitor on the specific biological or biochemical function in question.

As used herein, the term “avidity” refers to the overall strength of binding interactions between a protein receptor and its ligand. Avidity generally refers to the accumulated strength of multiple, individual non-covalent binding interactions, such as between a protein receptor and its ligand, and is distinct from “affinity”, which describes the strength of a single binding interaction. It should be understood that avidity is rarely the mere sum of its constituent affinities as many factors (such as local concentration or proximity, multimerization, 3D structure or conformation, etc.) can affect biomolecular interactions.

As used herein, the term “functionally equivalent” refers to variant sequences that have the same or substantially the same biological activity or function as the original sequence from which it is derived, e.g., no significant change in physiological, chemical, physico-chemical or functional properties compared to the original sequence. The term “substantially identical” refers to sequences that are functionally equivalent to the original or reference sequence and have a high degree of sequence identity thereto. Generally, a substantially identical sequence is at least about 80%, at least about 85%, at least about 90%, at least about 95%, at least about 96%, at least about 97%, at least about 98%, or at least about 99% identical to the original or reference sequence and has the same function. In some cases when referring to nucleic acid sequences, a substantially identical sequence hybridizes to the original sequence under high stringency conditions, for example at salt and temperature conditions substantially equivalent to 0.5 X SSC to about 5 X SSC and 65° C. for both hybridization and wash. In general, variant sequences that are substantially identical or functionally equivalent to sequences provided in accordance with the present disclosure are meant to be encompassed.

As used herein, the term “multimerization domain” refers to an amino acid sequence that allows polypeptide chains to assemble into a multimer. The term “multimer” refers to a molecule made from multiple monomers. The term “multimer” encompasses, without limitation, dimers, trimers, 4-mers, 5-mers, 6-mers, 8-mers, 10-mers, etc.

The term “dimeric” refers to the presence of two polypeptides as described herein in the TGFβ binding agent. “Homodimeric” means the two polypeptides have the same sequence, whereas “heterodimeric” means the two polypeptides have different sequences.

The term “doublet” refers to the presence of two copies of the TGFβR ligand binding domain (LBD) linked together in tandem in the polypeptide.

The term “tetravalent” refers to the presence of fours copies of TGFβR ligand binding domain (LBD) in the TGFβ binding agent.

### Polypeptides and TGFβ Binding Agents

There are provided herein novel polypeptide constructs comprising a TGFβ binding region and a multimerization domain, and TGFβ binding agents comprising two such polypeptide constructs assembled via the multimerization domain. The TGFβ binding region comprises two TGFβRII-LBDs linked in tandem by a first linker, and it is linked to the multimerization domain by a second linker. Polypeptide constructs and TGFβ binding agents of the present disclosure are optimized by improving their association with TGFβ. Specifically, the linkers are selected to optimize TGFβ isoform specificity such that the TGFβ3:TGFβ1 IC₅₀ ratio is no more than about 2.5:1 (indicating similar inhibition potency for both isoforms), without increasing undesired inhibition of TGFβ2, and without significantly reducing the overall potency (e.g., IC₅₀ remains in the picomolar range).

In exemplary embodiments, the polypeptide constructs and TGFβ binding agents of the present disclosure include two polypeptide chains that are associated via an Fc region of an antibody or via a constant CH2 domain, a constant CH3 domain and/or via a combination of CH2 and CH3. The constant region of the antibody may be from a human IgG1, IgG2, IgG3 or IgG4 antibody, or substantially identical thereto. The association of both polypeptide chains generally occurs during expression and secretion of the protein, e.g. in mammalian cells. In some exemplary embodiments, TGFβ binding agents may comprise homodimers, i.e., dimers of a polypeptide construct having the sequence set forth in any one of SEQ ID NOs: 81 to 103 and 105, or a sequence at least 80%, at least 85%, at least 90%, at least 95%, at least 96%, at least 97%, at least 98%, or at least 99% identical thereto. In other embodiments, TGFβ binding agents comprise heterodimers, i.e., dimers of two different polypeptide constructs, at least one of the polypeptide constructs having the sequence set forth in any one of SEQ ID NOs: 81 to 103 and 105, or a sequence at least 80%, at least 85%, at least 90%, at least 95%, at least 96%, at least 97%, at least 98%, or at least 99% identical thereto.

In general, polypeptide constructs and TGFβ binding agents are organized such that the multimerization domain is linked at its N-terminus to the C-terminus of the TGFβ binding region, so that for each polypeptide, the orientation of the construct is, from N-terminus to C-terminus, a single chain of (N-terminal region)-(first TGFβR-LBD)-(first linker)-(second TGFβR-LBD)-(second linker)-multimerization domain.

In an exemplary embodiment, the multimerization domain allows assembly of two or more polypeptide chains in a covalent manner, for example by disulfide linking between cysteine residues. Alternatively, the multimerization domain may allow polypeptide chains to be assembled in a non-covalent manner such as, for example and without limitation, by coiled-coil structure (De Crescenzo, G. et al., 2004).

In an embodiment, the multimerization domain is a dimerization domain, i.e., allows assembly of two polypeptide chains, to form a dimer. In accordance with the present disclosure, such dimers generally comprise two polypeptides, each polypeptide including two TGFβR-LBDs linked together and linked to the dimerization domain as described herein, thereby forming a tetravalent TGFβ binding agent. Homodimers and heterodimers of polypeptide constructs provided herein are encompassed.

In some embodiments, the multimerization or dimerization domain of the polypeptide comprises constant regions of an immunoglobulin heavy chain, including for example a CH2 and/or CH3 domain. An Fc portion of an immunoglobulin is typically used. However, a coiled-coil structure has also been found to be suitable for dimerization. Exemplary embodiments of Fc portions include, for example and without limitation, those that have lost their ability to interact with a particular Fc receptor. In additional embodiments, the multimerization domain may comprise an IgG-like dimerization domain, e.g., an IgG1, IgG2, IgG3, or IgG4 dimerization domain. In some embodiments, the multimerization domain may provide one or more effector function such as antibody dependent cellular cytotoxicity (ADCC), complement activation (complement dependent cytotoxicity, CDC), or opsonization.

In some embodiments, the multimerization or dimerization domain comprises a CH2, a CH3, or a CH2 and a CH3 from an antibody heavy chain that is of human origin. For example, and without wishing to be limiting, the antibody heavy chain may be selected from the group consisting of a human IgG1, IgG2, IgG3, or IgG4. In embodiments, the constant domain in the constructs is CH2 per se, or CH3 per se, or CH2-CH3. The antibody heavy chain component typically provides for disulfide crosslinking between single chain polypeptide constructs that are the same or different. In an embodiment, the multimerization domain provides for at least one disulfide link between single chain polypeptide constructs. In another embodiment, the multimerization domain provides for at least two disulfide links between single chain polypeptide constructs. In some cases, the antibody heavy chain also provides for protein A-based isolation of the dimeric polypeptide, e.g. after production in host cells.

Thus in some embodiments, the multimerization or dimerization domain is an antibody constant domain that provides for cross-linking between two of the present polypeptide constructs. This is achieved when, for example, expressed polypeptide constructs are secreted from their expression host. Thus, production of a single chain polypeptide may provide the construct in a dimeric form in which the two polypeptide chains are cross-linked via disulfide bridges that involve one or more cysteine residues within each of the antibody constant domains present in each of the polypeptides. In some embodiments, the multimerization domain (e.g., the constant region) has no particular activity, other than to act as a structure through which multimers (e.g., dimers) can form. Such minimal constant regions can also be altered to provide some benefit, by incorporating the corresponding hinge regions and optionally changing the cysteine residue composition. For example, some or all of the cysteine residues involved in bridging the two Fc fragments or naturally used to bridge between the heavy and light chains of a full-length antibody can be replaced or deleted. One advantage of minimizing the number of cysteine residues is to reduce the propensity for disulfide bond scrambling, which could promote aggregation. It should be noted that not all of the naturally-occurring inter-hinge disulfide bonds need to be formed for Fc dimerization to occur, while noting that the stability of the Fc dimer may depend on the number of inter-molecular disulfide bridges.

As used herein, the terms “antibody” and “immunoglobulin (Ig)” are used interchangeably to refer to a protein constructed from paired heavy and light polypeptide chains. The structures of an antibody and of each of the domains are well established and familiar to those of skill in the art, and are summarized only briefly here. When an antibody is correctly folded, each chain folds into a number of distinct globular domains joined by more linear polypeptide sequences. In particular, the Ig light chain folds into a variable (VL) and a constant (CL) domain, while the heavy chain folds into a variable (VH) and three constant (CH1, CH2, CH3) domains. Once paired, interaction of the heavy and light chain variable domains (VH and VL) and first constant domain (CL and CH1) results in the formation of a Fab (Fragment, antigen-binding) containing the binding region (Fv); interaction of two heavy chains results in pairing of CH2 and CH3 domains, leading to the formation of a Fc (Fragment, crystallisable). Characteristics described herein for the CH2 and CH3 domains also apply to the Fc.

In certain embodiments and aspects of the present disclosure, the multimerization or dimerization domain may have at least about 70%, at least about 75%, at least about 80%, at least about 85%, at least about 90%, at least about 95%, at least about 96%, at least about 97%, at least about 98%, at least about 99% or 100% sequence identity with an IgG1, IgG2, IgG3 or IgG4 constant region or with the CH2 and/or CH3 domain. The IgG1, IgG2, IgG3 or IgG4 may be from a human. In a particular embodiment, the TGFβ binding agents described herein include those having a dimerization domain of an IgG1. In another particular embodiment, the TGFβ binding agents described herein include those having a dimerization domain of an IgG4.

A multimerization or dimerization domain may be engineered to reduce aggregation or to modulate stability of a TGFβ binding agent formed by the assembly of more than one polypeptide disclosed herein. Fc portions having mutation(s) in, e.g., the hinge region are therefore encompassed by the present disclosure. Exemplary embodiments of Fc variants and modified hinge regions are provided for example in patent applications published under Nos. WO2018/158727 and WO2017/037634. It should be understood that, when the hinge portion of a multimerization or dimerization domain is referenced, the hinge is part of the multimerization domain and is not considered part of the second linker.

In exemplary embodiments, multimerization or dimerization domains have the sequence set forth in SEQ ID NOs: 49-80, or a functionally equivalent variant thereof, or a sequence at least about 80%, at least about 90%, at least about 95%, at least about 96%, at least about 98%, or at least about 99% identical thereto. In a particular embodiment, the multimerization domain may comprise SEQ ID NO: 49 or a sequence at least about 80%, at least about 90%, at least about 95%, at least about 96%, at least about 98%, or at least about 99% identical thereto. In another embodiment, the multimerization domain may comprise SEQ ID NO: 50 or a sequence at least about 80%, at least about 90%, at least about 95%, at least about 96%, at least about 98%, or at least about 99% identical thereto.

In accordance with the present disclosure, the first multimerization or dimerization domain and the second multimerization or dimerization domain of a TGFβ binding agent may have the same or substantially the same amino acid sequence in certain embodiments. Alternatively, in some embodiments the multimerization or dimerization domain may be different, as long as multimerization is not adversely affected.

It should be understood that the multimerization domain is not meant to be particularly limited. Any amino acid sequence that allows association of the polypeptide chains to form a tetravalent TGFβ binding agent in accordance with the present disclosure may be used, as long as the desired function and isoform specificity is maintained.

In accordance with the present disclosure, linkers in the polypeptide constructs and TGFβ binding agents may comprise or consist of an IDR linker, an IDR linker variant, a hybrid linker, a hybrid linker variant, a truncated linker, a truncated linker variant or an elongated linker, as disclosed herein.

The human TGFβRII ectodomain (TGFβRII-ECD; SEQ ID NO: 1) includes a 102 amino acid structured ligand-binding domain (SEQ ID NO: 2; also referred to herein as “TGFβR-LBD”) that is flanked by two intrinsically disordered regions: a region of 24 amino acids at the N-terminal (SEQ ID NO: 3) and a region of 10 amino acids at the C-terminal (SEQ ID NO: 4).

As used herein, the term “intrinsically disordered region (IDR) linker” refers to a linker comprising or consisting of at least a portion of one or both of the intrinsically disordered regions (IDRs) that flank the structured, ligand-binding domain of the TGFβRII ectodomain. An IDR linker generally possesses substantial sequence identity with at least one sequence of an intrinsically disordered region of the TGFβRII ectodomain, and it may possess substantial sequence identity with both the N- and C-terminal IDRs of the TGFβRII ectodomain or portions thereof. It should be understood that an IDR linker may comprise the entire IDR of the TGFβR or only a portion thereof, or multiple portions linked together.

In some embodiments, an IDR linker comprises or consists of a portion of one or both of the IDRs (SEQ ID NOs: 3 and 4) of the human TGFβRII-ECD (SEQ ID NO: 1). In embodiments where a portion of each of the two IDRs of the TGFβR is included, the portions may be linked together either directly or via an intervening linker sequence. The portions of the IDRs may include the entire IDR sequence or variants (e.g., substitutions, truncations) thereof.

In one embodiment, an IDR linker comprises or consists of a portion of each IDR linked directly together. In certain embodiments, the C-terminal IDR or a portion thereof is linked directly to the N-terminus of the N-terminal IDR or a portion thereof. Non-limiting examples of such embodiments include, for example, linkers having the amino acid sequence set forth in SEQ ID NOs: 8-16, 19, 22, 23, and 26.

In one embodiment, an IDR linker comprises or consists of the sequence set forth in SEQ ID NO: 4.

In one embodiment, an IDR linker does not consist of the sequence set forth in SEQ ID NO: 7. In such embodiments, polypeptides and TGFβ binding agents comprising the sequence set forth in SEQ ID NO: 7 are excluded from the present invention. In some embodiments, IDR linkers that do not provide the desired isoform specificity (e.g., the desired TGFβ3:TGFβ1 IC₅₀ ratio) are excluded from the present invention, as are polypeptides and TGFβ binding agents comprising such sequences.

IDR linker variants, hybrid linkers, hybrid linker variants, truncated linkers, truncated linker variants and elongated linkers are derived from the sequences of IDR linkers disclosed herein.

As used herein the term “non-IDR linker” means a linker that does not share substantial homology or identity with the intrinsically disordered regions (IDRs) that flank the structured, ligand-binding domain of the TGFβRII ectodomain. In aspects and embodiments described herein, the non-IDR linker may be a flexible linker, including for example, and without limitation glycine and glycine-serine (GS) linkers. It is a common practice when producing fusion constructs to introduce artificial, highly flexible glycine or glycine-serine linkers such as GGGGS or [G4S]n (where n is 1, 2, 3, 4 or 5 or more, such as 10, 25 or 50) between the various regions of the constructs. However, such artificial linkers can also be disadvantageous due to their potential for undesired immunogenicity and their added molecular weight. Entropic factors are also a potential liability for glycine and GS linkers, which are highly flexible and may become partially restricted upon target binding, causing a loss of entropy that disfavors binding. Therefore in some embodiments, polypeptides and TGFβ binding agents of the present disclosure do not include a non-IDR linker, or include at least one IDR linker or IDR linker variant in addition to the non-IDR linker. Non-limiting examples of non-IDR linkers in accordance with the present disclosure include SEQ ID NOs: 17, 20, 21 and 24.

In some embodiments, a linker comprises or consists of a mixture of an IDR and a GS linker, such as, for example, the amino acid sequence set forth in SEQ ID NOs: 18 and 25. Such linkers are referred to herein as hybrid linkers. In some embodiments, there are provided linkers in which from 3 to 7 or from 3 to 14 amino acid residues in any one of SEQ ID NOs: 4, 8-16, 19, 22, 23, and 26 have been replaced with an amino acid sequence comprising glycine and/or serine residues (a glycine or a GS linker). These linkers are referred to herein as hybrid linkers. Hybrid linker variants are also encompassed; these are functionally equivalent variants of hybrid linkers that include one or more insertion, deletion, or amino acid substitution, optionally a conservative amino acid substitution. Variants are discussed further below.

In exemplary embodiments of hybrid linkers and hybrid linker variants, at least 3 consecutive amino acids of IDR linkers or IDR linker variants are replaced with glycine and/or serine residues. In further exemplary embodiments of hybrid linkers or hybrid linker variants, at least 7 consecutive amino acids of IDR linkers or IDR linker variants are replaced with glycine and/or serine residues. In yet further exemplary embodiments of hybrid linkers or hybrid linker variants, two sets of from 3 to 7 consecutive amino acids of IDR linkers or IDR linker variants are replaced with glycine and/or serine residues. The two sets of 3 to 7 consecutive amino acids may be spaced within a linker sequence or may be consecutive.

Examples of glycine and GS sequences for use in hybrid linkers and hybrid linker variants include, without limitation, GSG, and any one of SEQ ID NOs: 17, 18, 20, 21, 24, and 25.

In some embodiments, a linker is a truncated linker or a truncated linker variant. Such linkers have a truncation (deletion) of, for example, from 1 to about 20 consecutive amino acids (and any range comprised within 1 and about 20 such as, for example, from 1 to about 10, 1 to about 5, etc.) at either or both the N- or C-terminus of an IDR linker provided herein. In an exemplary embodiment, the amino acid truncation may be at the N-terminus of any one of SEQ ID NO:3 or 8-26. In an exemplary embodiment the truncation may result in the removal of 1, 2, 3, 4, 5, 6, 7, 8, 9, 10, 11, 12, 13, 14, 15, 16, 17, 18, 19 or 20 residues at the N-terminus. In another exemplary embodiment, the truncation may be at the C-terminus of any one of SEQ ID NOs: 4 and 8-26. In an exemplary embodiment the truncation may result in the removal of 1, 2, 3, 4, 5, 6, 7, 8, or 9 residues at the C-terminus. In another embodiment, the truncation may result in the removal of 1, 2, 3, 4, 5, 6, 7, 8, 9, 10, 11, 12, 13, 14, 15, 16, 17, 18, 19 or 20 residues at the C-terminus.

In another embodiment, the truncation may be an internal deletion such as for example a deletion starting at amino acid number 10 or 11 of SEQ ID NO: 7. In an embodiment, 1, 2, 3, 4, 5, 6, 7, 8, 9, 10, 11, 12, 13, 14, 15, 16, 17, 18, 19 or 20 residues are deleted from SEQ ID NO: 7, including amino acid number 10 and/or 11. In other embodiments, 1, 2, 3, 4, 5, 6, 7, 8, 9, 10, 11, 12, 13, 14, 15, 16, 17, 18, 19 or 20 residues are deleted internally from any one of SEQ ID NOs: 4 and 8-26.

In an exemplary embodiment, the amino acid truncation may result in the removal of from 1 to 10 amino acids encompassing the region defined by amino acid residues numbers 11 to 20 of any one of SEQ ID NOs: 7-26.

Other exemplary and non-limiting embodiments of truncated linkers are provided in SEQ ID NOs: 8-16, 18, 19, 22, 23, and 26.

The present disclosure further provides truncated linker variants. Such truncated linker variants may comprise an amino acid substitution (conservative or non-conservative) in comparison to the truncated linkers disclosed herein.

In some embodiments, the first linker comprises or consists of an amino acid sequence having: (a) a deletion of at least one N-terminal amino acid residue in comparison with SEQ ID NO: 3, SEQ ID NO: 7, SEQ ID NO: 12 or SEQ ID NO: 8; (b) a deletion of at least one C-terminal amino acid residue in comparison with SEQ ID NO: 3, SEQ ID NO: 7, SEQ ID NO: 12 or SEQ ID NO: 8; (c) a deletion of at least one internal amino acid residue in comparison with SEQ ID NO: 3, SEQ ID NO: 7, SEQ ID NO: 12 or SEQ ID NO: 8; or (d) one or more substitution in the amino acid sequence in comparison with SEQ ID NO: 3, SEQ ID NO: 7, SEQ ID NO: 12, SEQ ID NO: 8, or of any one of (a) to (c). In an embodiment thereof, the amino acid deletion is a deletion of 16 amino acids of SEQ ID NO: 3.

In some embodiments, the second linker comprises or consists of an amino acid sequence having: (a) a deletion of at least one N-terminal amino acid residue in comparison with SEQ ID NO: 7 or SEQ ID NO: 9 or SEQ ID NO: 11; (b) a deletion of at least one C-terminal amino acid residue in comparison with SEQ ID NO: 7 or SEQ ID NO: 9 or SEQ ID NO: 11; (c) a deletion of at least one internal amino acid residue in comparison with SEQ ID NO: 7 or SEQ ID NO: 9 or SEQ ID NO: 11; or (d) one or more substitution in the amino acid sequence in comparison with SEQ ID NO: 4, 7, 9 or 11, or of any one of (a) to (c).

In some embodiments, a linker is an elongated linker. Such linkers have an addition (elongation) of from 1 to 10 amino acids (and any range comprised within 1 and 10 such as for example, from 1 to 7, from 1 to 5, from 1 to 3, 1, 2, 3 etc.) at either or both the N- or C-terminus of any of an IDR linker, an IDR linker variant, a hybrid linker, a hybrid linker variant, a truncated linker, or a truncated linker variant, as disclosed herein. These additional amino acids may each independently be selected from any amino acid residue.

In an exemplary embodiment, the linkers disclosed herein may comprise from 1 to 5 additional amino acid residues at their N-terminus. In another exemplary embodiment, the linkers disclosed herein may comprise from 1 to 5 additional amino acid residues at their C-terminus. In a further exemplary embodiment, the linkers disclosed herein may comprise from 1 to 5 additional amino acid residues at both their N-terminus and C-terminus. Such additional amino acid residues may be selected from any amino acid residues and may be either the same or different. Other exemplary and non-limiting embodiments of elongated linkers encompass addition of from 1 to 10 amino acids (and any range comprised within 1 and 10 such as for example, from 1 to 7, from 1 to 5, from 1 to 3, 1, 2, 3 etc.) at either or both the N- or C-terminus of any one of SEQ ID NOs: 4 and 8-26. The added sequence may comprise any amino acid residues.

Exemplary embodiments of elongated linkers also include those comprising a non-IDR linker portion at either or both of its N- or C-terminus. For example, the IDR linker, the IDR linker variant, the hybrid linker, the hybrid linker variant, the truncated linker or the truncated linker variant may be flanked by at least one non-IDR linker at either or both of its N-and C-terminus. Alternatively, a non-IDR linker may be flanked by at least the IDR linker, the IDR linker variant, the hybrid linker, the hybrid linker variant, the truncated linker or the truncated linker variant at either or both of its N- and C-terminus.

In some embodiments of the polypeptide constructs and TGFβ binding agents of the present disclosure, the N-terminal region comprises or consists of the N-terminal IDR (SEQ ID NO: 3) in the TGFβRII-ECD (SEQ ID NO: 1), or a sequence substantially identical thereto, such as without limitation a truncated or substituted variant thereof. It should be understood that the N-terminal region may be truncated and/or substituted and otherwise modified, as long as desired inhibition potency and specificity are not adversely affected.

The present disclosure also encompasses variants of the polypeptides and the TGFβ binding agents described herein. Variants encompassed by the present disclosure include those having a variation in the amino acid sequence of any one of the elements (first and second TGFβ receptor ligand-binding domain (TGFβR-LBD), first linker, second linker, N-terminal region, multimerization domain, etc.) of the polypeptide or TGFβ binding agent. Variants of the polypeptide or TGFβ binding agent include, for example, those having similar or improved binding affinity, avidity, isoform-specificity, potency of inhibition, stability, manufacturability, and/or reduced aggregation in comparison with the polypeptides and TGFβ binding agents disclosed herein.

A site of interest for substitutional mutagenesis includes the multimerization domain of the polypeptide or TGFβ binding agent. Exemplary embodiments of polypeptide or TGFβ binding agent variants of the present disclosure may comprise those having a modified IgG1, IgG2, IgG3, or IgG4 constant region or a portion thereof. TGFβ binding agents that may comprise an IgG1 constant region (modified or unmodified) are encompassed herewith. TGFβ binding agents that may comprise an IgG4 constant region (modified or unmodified) are also encompassed herewith.

Variants encompassed by the present disclosure include those which may comprise an insertion, a deletion or an amino acid substitution (conservative or non-conservative). These variants may have at least one amino acid residue in its amino acid sequence removed and a different residue inserted in its place.

In general, a conservative amino acid substitution is the substitution of an amino acid residue for another amino acid residue with similar chemical properties (e.g., size, charge, or polarity). Conservative substitutions may be made by exchanging an amino acid from one of the groups listed below (group 1 to 6) for another amino acid of the same group.

Other exemplary embodiments of conservative substitutions are shown in Table 1 under the heading of “preferred substitutions”. If such substitutions result in an undesired property, then more substantial changes, denominated “exemplary substitutions” in Table 1, or as further described below in reference to amino acid classes, may be introduced and the products screened.

It is known in the art that variants may be generated by substitutional mutagenesis and retain the biological activity (i.e., functional equivalence) of the polypeptides of the present disclosure. These variants have at least one amino acid residue in the amino acid sequence removed and a different residue inserted in its place, e.g., one or more conservative amino acid substitution. Examples of substitutions identified as “conservative substitutions” are shown in Table 1. If such substitutions result in a change not desired, then other types of substitutions, denominated “exemplary substitutions” in Table 1, or as further described herein in reference to amino acid classes, are introduced and the products screened.

Amino acid residues may be divided into groups based on common side chain properties, as follows:
(group 1) hydrophobic: norleucine, methionine (Met), Alanine (Ala), Valine (Val), Leucine (Leu), Isoleucine (Ile) ;
(group 2) neutral hydrophilic: Cysteine (Cys), Serine (Ser), Threonine (Thr), Asparagine (Asn), Glutamine (Gln);
(group 3) acidic: Aspartic acid (Asp), Glutamic acid (Glu);
(group 4) basic: Histidine (His), Lysine (Lys), Arginine (Arg);
(group 5) residues that influence chain orientation: Glycine (Gly), Proline (Pro); and
(group 6) aromatic: Tryptophan (Trp), Tyrosine (Tyr), Phenylalanine (Phe).

Non-conservative substitutions will entail exchanging a member of one of these classes for another.

TABLE 1

Exemplary amino acid substitutions.

| | | |
|---|---|---|
| Original residue | Exemplary substitution | Conservative substitution |
| Ala (A) | Val, Leu, Ile | Val |
| Arg (R) | Lys, Gln, Asn | Lys |
| Asn (N) | Gln, His, Lys, Arg, Asp | Gln |
| Asp (D) | Glu, Asn | Glu |
| Cys (C) | Ser, Ala | Ser |
| Gln (Q) | Asn; Glu | Asn |
| Glu (E) | Asp, Gln | Asp |
| Gly (G) | Ala | Ala |
| His (H) | Asn, Gln, Lys, Arg, | Arg |
| Ile (I) | Leu, Val, Met, Ala, Phe, norleucine | Leu |
| Leu (L) | Norleucine, Ile, Val, Met, Ala, Phe | Ile |
| Lys (K) | Arg, Gln, Asn | Arg |
| Met (M) | Leu, Phe, Ile | Leu |
| Phe (F) | Leu, Val, Ile, Ala, Tyr | Tyr |
| Pro (P) | Ala | Ala |
| Ser (S) | Thr | Thr |
| Thr (T) | Ser | Ser |
| Trp (W) | Tyr, Phe | Tyr |
| Tyr (Y) | Trp, Phe, Thr, Ser | Phe |
| Val (V) | Ile, Leu, Met, Phe, Ala, norleucine | Leu |

Generally, the degree of similarity and identity between variable chains is determined herein using the Blast2 sequence program (Tatusova, T.A. and Madden, T.L., 1999) using default settings, i.e., blastp program, BLOSUM62 matrix (open gap 11 and extension gap penalty 1; gapx dropoff 50, expect 10.0, word size 3) and activated filters.

However, the level of identity may also be determined over the entire length of a given sequence. Percent identity will therefore be indicative of amino acids which are identical in comparison with the original peptide and which may occupy the same or similar position. Percent similarity will be indicative of amino acids which are identical and those which are replaced with conservative amino acid substitution in comparison with the original peptide at the same or similar position.

In some embodiments, variants of the present disclosure therefore comprise amino acid sequences which have at least 50%, at least 60%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity with an original sequence or a portion of an original sequence.

In some embodiments, variation in the amino acid sequence occurs in the TGFβ receptor ligand-binding domain (TGFβR-LBD) of the polypeptide or TGFβ binding agent. In other embodiments, variation may occur outside of the TGFβ receptor ligand-binding domain (TGFβR-LBD) of the TGFβ binding agent. Variants encompassed by the present disclosure may have a TGFβR-LBD that is identical or substantially identical to the structured ligand-binding domain found in the ectodomain (ECD) of TGFβ receptors (including in human, animals etc.). In further embodiments, variation in the amino acid sequence occurs in the multimerization domain. In still other embodiments, variation in the amino acid sequence occurs in the first and/or second linker. It should be understood that variation may occur in multiple regions of the polypeptide or TGFβ binding agent, as long as the desired function is maintained.

In some embodiments, the polypeptide or TGFβ binding agent of the present disclosure may be conjugated, for example with a targeting agent, a therapeutic moiety (for therapeutic purposes) or with a detectable moiety (i.e., for detection or diagnostic purposes).

In an exemplary embodiment, the polypeptide or TGFβ binding agent of the present disclosure is conjugated with a therapeutic moiety such as, for example and without limitation, a chemotherapeutic, a cytokine, a cytotoxic agent, an anti-fibrotic drug, an anti-cancer drug (e.g., small molecule), a single chain antibody, and the like.

In another exemplary embodiment, the polypeptide or TGFβ binding agent of the present disclosure is conjugated with a detectable moiety including, for example and without limitation, a moiety detectable by spectroscopic, photochemical, biochemical, immunochemical, chemical and/or other physical means. A detectable moiety may be coupled either directly or indirectly (for example via a linkage, such as, without limitation, a DOTA or NHS linkage) to the TGFβ binding agent using methods well known in the art. A wide variety of detectable moieties may be used, with the choice depending on the sensitivity required, ease of conjugation, stability requirements and available instrumentation. A suitable detectable moiety may include, but is not limited to, a fluorescent label, a radioactive label (for example, without limitation, ¹²⁵I, In¹¹¹, Tc⁹⁹, I¹³¹ and including positron emitting isotopes for PET scanner etc), a nuclear magnetic resonance active label, a luminescent label, a chemiluminescent label, a chromophore label, an enzyme label (for example and without limitation horseradish peroxidase, alkaline phosphatase, etc.), quantum dots and/or a nanoparticle. A detectable moiety may cause and/or produce a detectable signal thereby allowing for a signal from the detectable moiety to be detected.

A therapeutic moiety may include, for example and without limitation, Yttrium-90, Scandium-47, Rhenium-186, Iodine-131, Iodine-125, and many others recognized by those skilled in the art (e.g., lutetium (e.g., Lu¹⁷⁷), bismuth (e.g., Bi²¹³), copper (e.g., Cu⁶⁷)), 5-fluorouracil, adriamycin, irinotecan, taxanes, pseudomonas endotoxin, ricin, auristatins (e.g., monomethyl auristatin E, monomethyl auristatin F), maytansinoids (e.g., mertansine), and other toxins.

In another embodiment, a therapeutic moiety may include another therapeutic for a TGFβ-associated disease or condition. For example and without limitation, one or more polypeptide construct or TGFβ binding agent may be linked to a cytotoxic drug in order to generate an antibody-drug conjugate (ADC).

A targeting agent may include, for example, an amino acid sequence for delivering the polypeptide or TGFβ binding agent to a desired tissue, organ or location in a subject’s body. For example and without limitation, a targeting agent may comprise a poly-aspartate sequence motif for bone targeting, or an antibody or antigen-binding fragment.

In other exemplary embodiments, a targeting agent, therapeutic moiety or diagnostic moiety may comprise, for example and without limitation, an antibody or antigen binding fragment thereof (e.g., single chain antibody), a binding agent having affinity for another member of the TGFβ family or for another therapeutic target, a radiotherapy agent, an imaging agent, a fluorescent moiety, a cytotoxic agent, an anti-mitotic drug, a nanoparticle-based carrier, a polymer-conjugated to drug, nanocarrier, imaging agent, a stabilizing agent, a drug, a nanocarrier and/or a dendrimer.

It should be understood that the site for conjugation is not particularly limited, as long as the function of the polypeptide or TGFβ binding agent is not adversely affected. For example and without limitation, a targeting agent, therapeutic moiety or detectable moiety may be conjugated in the linker portion of a polypeptide or TGFβ binding agent (e.g., in a non-IDR linker) or at any other suitable site such as at its N-terminus or in the multimerization domain.

### Production of Polypeptides and TGFβ Binding Agents

The polypeptide or TGFβ binding agent disclosed herein may be made by a variety of methods familiar to those skilled in the art, including by recombinant DNA methods.

In order to express the polypeptides or TGFβ binding agents, nucleotide sequences able to encode the polypeptide chain described herein may be inserted into an expression vector, i.e., a vector that contains the elements for transcriptional and translational control of the inserted coding sequence in a particular host. These elements may include regulatory sequences, such as enhancers, constitutive and inducible promoters, and 5′ and 3′ un-translated regions. Methods that are well known to those skilled in the art may be used to construct such expression vectors. These methods include in vitro recombinant DNA techniques, synthetic techniques, in vivo genetic recombination and the like.

A variety of expression vector and host cell systems known to those of skill in the art may be used to express the polypeptide chains described herein. These include, but are not limited to, microorganisms such as bacteria transformed with recombinant bacteriophage, plasmid, or cosmid DNA expression vectors; yeast transformed with yeast expression vectors; insect cell systems infected with baculovirus vectors; plant cell systems transformed with viral or bacterial expression vectors; and animal cell systems. For long-term production of recombinant proteins in mammalian systems, stable expression in mammalian cell lines may be used. For example, nucleotide sequences able to encode any one of the polypeptide chains described herein may be transformed into cell lines using expression vectors that may contain viral origins of replication and/or endogenous expression elements and a selectable or visible marker gene on the same or on a separate vector. The present disclosure is not to be limited by the vector or host cell employed. In certain embodiments disclosed herein, nucleic acids able to encode polypeptide chains described herein may be ligated into expression vectors. In the event that the TGFβ binding agent is composed of distinct polypeptide chains (i.e., the first polypeptide and the second polypeptide are not identical), each of such polypeptide chain may be ligated into separate vectors or into the same vector. In accordance with the present disclosure, the polypeptide chains of the TGFβ binding agent may be encoded by a single vector or by separate vectors (e.g., a vector set). Cells are transformed with the desired vector or vector sets.

Alternatively, the polypeptide chains may be expressed from an in vitro transcription system or a coupled in vitro transcription/translation system respectively or any such cell-free system.

Host cells comprising nucleotide sequences may be cultured under conditions for the transcription of the corresponding RNA (mRNA, etc.) and/or the expression and secretion of the polypeptide(s) from cell culture. In an exemplary embodiment, expression vectors containing nucleotide sequences able to encode the polypeptide chains described herein may be designed to contain signal sequences that direct secretion of the polypeptide through a prokaryotic or eukaryotic cell membrane.

Due to the inherent degeneracy of the genetic code, DNA sequences that encode the same, substantially the same or a functionally equivalent amino acid sequence may be produced and used. The nucleotide sequences of the present disclosure may be engineered using methods generally known in the art in order to alter the nucleotide sequences for a variety of purposes including, but not limited to, modification of the cloning, processing, and/or expression of the gene product. DNA shuffling by random fragmentation and PCR reassembly of gene fragments and synthetic oligonucleotides may be used to engineer the nucleotide sequences. For example, oligonucleotide-mediated site-directed mutagenesis may be used to introduce mutations that create new restriction sites, alter glycosylation patterns, change codon preference, produce splice variants, and so forth. Codon-optimized nucleic acids encoding the polypeptide chains described herein are encompassed by the present disclosure.

In addition, a host cell strain may be chosen for its ability to modulate expression of the inserted sequences or to process the expressed polypeptide in the desired fashion. Different host cells that have specific cellular machinery and characteristic mechanisms for post-translational activities (e.g., CHO, HeLa, MDCK, HEK293, and W138) are available commercially and from the American Type Culture Collection (ATCC) and may be chosen to ensure the correct modification and processing of the expressed polypeptide.

Those of skill in the art will also readily recognize that the nucleic acid and polypeptide sequences may be synthesized, in whole or in part, using chemical or enzymatic methods well known in the art. For example, peptide synthesis may be performed using various solid-phase techniques and machines such as the ABI 431A Peptide synthesizer (PE Biosystems) may be used to automate synthesis. If desired, the amino acid sequence may be altered during synthesis and/or combined with sequences from other proteins to produce a variant protein.

### Pharmaceutical Compositions

Pharmaceutical compositions comprising the polypeptides or TGFβ binding agents disclosed herein are also encompassed by the present disclosure. The pharmaceutical composition generally comprises the polypeptide or TGFβ binding agent disclosed herein and a pharmaceutically acceptable carrier.

The preparation of pharmaceutical compositions can be carried out as known in the art (see, for example, Remington: The Science and Practice of Pharmacy, 20th Edition, 2000). For example, a therapeutic compound and/or composition, together with one or more solid or liquid pharmaceutical carrier substances and/or additives (or auxiliary substances) and, if desired, in combination with other pharmaceutically active compounds having therapeutic or prophylactic action, are brought into a suitable administration form or dosage form which can then be used as a pharmaceutical in human or veterinary medicine. Pharmaceutical preparations can also contain additives, of which many are known in the art, for example fillers, disintegrants, binders, lubricants, wetting agents, stabilizers, emulsifiers, dispersants, preservatives, sweeteners, colorants, flavorings, aromatizers, thickeners, diluents, buffer substances, solvents, solubilizers, agents for achieving a depot effect, salts for altering the osmotic pressure, coating agents or antioxidants.

The term “pharmaceutical composition” means a composition comprising a polypeptide or TGFβ binding agent as described herein and at least one component comprising pharmaceutically acceptable carriers, diluents, adjuvants, excipients, or vehicles, such as preserving agents, fillers, disintegrating agents, wetting agents, emulsifying agents, suspending agents, sweetening agents, flavoring agents, perfuming agents, antibacterial agents, antifungal agents, lubricating agents and dispensing agents, depending on the nature of the mode of administration and dosage forms.

The term “pharmaceutically acceptable carrier” is used to mean any carrier, diluent, adjuvant, excipient, or vehicle, as described herein or as known in the art. Examples of suspending agents include ethoxylated isostearyl alcohols, polyoxyethylene sorbitol and sorbitan esters, microcrystalline cellulose, aluminum metahydroxide, bentonite, agar-agar and tragacanth, or mixtures of these substances. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monosterate and gelatin. Non-limiting examples of suitable carriers, diluents, solvents, or vehicles include water, salt solutions, phosphate buffered saline (PBS), gelatins, oils, alcohols, polyols, suitable mixtures thereof, vegetable oils (such as olive oil), and injectable organic esters such as ethyl oleate. Non-limiting examples of excipients include lactose, milk sugar, sodium citrate, calcium carbonate, and dicalcium phosphate. Non-limiting examples of disintegrating agents include starch, alginic acids, and certain complex silicates. Non-limiting examples of lubricants include magnesium stearate, sodium lauryl sulphate, talc, as well as high molecular weight polyethylene glycols.

The term “pharmaceutically acceptable” means it is, within the scope of sound medical judgment, suitable for use in contact with the cells of a subject, e.g., humans and animals, without undue toxicity, irritation, allergic response, and the like, and are commensurate with a reasonable benefit/risk ratio.

A pharmaceutically acceptable carrier may include any and all solvents, dispersion media, coatings, antibacterial and antifungal agents, isotonic and absorption delaying agents, and the like that are physiologically compatible. In one embodiment, the carrier is suitable for parenteral administration. The carrier may be suitable for intravenous, intraperitoneal, subcutaneous or intramuscular administration. Alternatively, the carrier may be suitable for sublingual or oral administration. In other embodiments, the carrier is suitable for topical administration or for administration via inhalation. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions provided herein is contemplated. Supplementary active compounds can also be incorporated into the compositions. For example, a pharmaceutical composition provided herein may further comprise at least one additional therapeutic agent, as discussed further below.

In some embodiments, a pharmaceutical composition provided herein can be administered orally, for example in the form of pills, tablets, lacquered tablets, sugar-coated tablets, granules, hard and soft gelatin capsules, aqueous, alcoholic or oily solutions, syrups, emulsions or suspensions, or rectally, for example in the form of suppositories.

In other embodiments, a pharmaceutical composition provided herein can be administered parenterally, for example subcutaneously, intramuscularly or intravenously in the form of solutions for injection or infusion. Other suitable administration forms are, for example, percutaneous or topical administration, for example in the form of ointments, creams, tinctures, sprays or transdermal therapeutic systems, or the inhalative administration in the form of nasal sprays or aerosol mixtures, or, for example, microcapsules, implants or wafers.

Pharmaceutical compositions typically must be sterile and stable under the conditions of manufacture and storage. A composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of injectable compositions can be brought about by including in the composition an agent which delays absorption, for example, monostearate salts and gelatin. Moreover, a compound can be administered in a time release formulation, for example in a composition which includes a slow release polymer. The compound can be prepared with carriers that will protect against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG).

Many methods for the preparation of such formulations are generally known to those skilled in the art. Sterile injectable solutions can be prepared by incorporating an active compound, such as a polypeptide or TGFβ binding agent provided herein, in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, common methods of preparation are vacuum drying and freeze-drying which yields a powder of the active ingredient plus any additional desired ingredient from a previously sterile-filtered solution thereof. Compounds may also be formulated with one or more additional compounds that enhance their solubility.

It is often advantageous to formulate compositions (such as parenteral compositions) in dosage unit form for ease of administration and uniformity of dosage. The term “unit dosage form” refers to a physically discrete unit suitable as unitary dosages for human subjects and other animals, each unit containing a predetermined quantity of active material calculated to produce the desired therapeutic effect, in association with a suitable pharmaceutical carrier. The specification for the dosage unit forms of the invention may vary and are dictated by and directly dependent on (a) the unique characteristics of the therapeutic compound and the particular therapeutic effect to be achieved, and (b) the limitations inherent in the art of compounding such a therapeutic compound for the prevention or treatment of a TGFβ associated disease or disorder. Dosages are discussed further below.

In some embodiments, there are provided pharmaceutical compositions that comprise an effective amount of a polypeptide and/or TGFβ binding agent described herein, and a pharmaceutically acceptable carrier. In an embodiment, there are provided pharmaceutical compositions for the treatment or prevention of fibrosis, comprising a polypeptide or TGFβ binding agent described herein, and a pharmaceutically acceptable carrier. In another embodiment, there is provided a pharmaceutical composition for the delay of progression of a cancer, for the inhibition of cancer invasion, e.g., malignant glial cell (MGC) invasion, for inhibition of cancer stem cell growth, survival, spheroid formation and/or proliferation, for inhibition of metastasis, for inhibition of cancer recurrence, and/or for overcoming chemoresistance of a cancer, the composition comprising a polypeptide and/or TGFβ binding agent described herein, and a pharmaceutically acceptable carrier. In another embodiment, there is provided a pharmaceutical composition for treating or preventing a bone marrow failure state.

As used herein “pharmaceutically acceptable carrier” or “pharmaceutical carrier” are known in the art and include, but are not limited to, 0.01-0.1 M or 0.05 M phosphate buffer or 0.8 % saline. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer’s dextrose, dextrose and sodium chloride, lactated Ringer’s orfixed oils. Intravenous vehicles include fluid and nutrient replenishers, electrolyte replenishers such as those based on Ringer’s dextrose, and the like. Preservatives and other additives may also be present, such as, for example, antimicrobials, antioxidants, collating agents, inert gases and the like.

For any compound, the therapeutically effective dose may be estimated initially either in cell culture assays or in animal models such as mice, rats, rabbits, dogs, or pigs. An animal model may also be used to determine the concentration range and route of administration. Such information may then be used to determine useful doses and routes for administration in humans. These techniques are well known to one skilled in the art and a therapeutically effective dose refers to that amount of active ingredient that ameliorates the symptoms or condition. Therapeutic efficacy and toxicity may be determined by standard pharmaceutical procedures in cell cultures or with experimental animals, such as by calculating and contrasting the ED₅₀ (the dose therapeutically effective in 50% of the population) and LD₅₀ (the dose lethal to 50% of the population). Any of the pharmaceutical compositions described herein may be applied to any subject in need of therapy, including, but not limited to, mammals such as dogs, cats, cows, horses, rabbits, monkeys, and especially humans.

The pharmaceutical compositions described herein may be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual, or rectal means.

### Methods of Use

The polypeptide or TGFβ binding agent described herein, and pharmaceutical compositions thereof, are useful for prevention or treatment of a TGFβ-associated disease or condition. As such, there are provided methods for prevention or treatment of a TGFβ-associated disease or condition in a subject, the methods comprising administering a therapeutically effective amount of the polypeptide, TGFβ binding agent or pharmaceutical composition described herein. Polypeptides and TGFβ binding agents are generally administered in the form of a pharmaceutical composition. A subject may be in need of such treatment, i.e., having, suspected of having, or at risk of having a disease or condition associated with TGFβ (e.g., TGFβ1 and/or TGFβ3).

As used herein, the term “TGFβ-associated disease or condition” refers to diseases or conditions that may be ameliorated through inhibition of TGFβ activity, particularly TGFβ1 and/or TGFβ3 activity. TGFβ-associated diseases or conditions include, without limitation, diseases or conditions associated with over-expression or over-activation of TGFβ ligands, particularly TGFβ1 and/or TGFβ3. In some embodiments, a TGFβ-associated disease or condition is mediated by TGFβ1 and/or TGFβ3. In one embodiment, the disease or condition to be treated is mediated by TGFβ3. In another embodiment, the disease or condition to be treated is mediated by a combination of TGFβ1 and TGFβ3. As used herein, the term “amelioration” means to decrease, suppress, attenuate, diminish, arrest, or stabilize the development or progression of a disease.

Examples of TGFβ-associated diseases or conditions that may be prevented or treated in accordance with the present disclosure include, without limitation: fibrosis (e.g., fibrotic disease, fibrotic scarring, fibroproliferative disorders); cancer (e.g., malignancies, solid tumors, metastasis); bone marrow failures (e.g., Shwachman-Bodian-Diamond syndrome, Fanconi anemia); ocular diseases; and genetic disorders of connective tissue.

In some embodiments, the polypeptide or TGFβ binding agent described herein is used for treatment or prevention of fibrosis, including for example and without limitation, fibrotic disease of tissues and/or organs, fibrotic scarring, and fibroproliferative disorders. Non-limiting examples of fibrotic diseases or conditions that may be treated or prevented include pulmonary fibrosis (e.g., idiopathic pulmonary fibrosis), renal fibrosis, liver fibrosis (e.g., hepatic cirrhosis), systemic sclerosis, scleroderma, skin fibrosis, heart fibrosis, bone marrow fibrosis, and myelofibrosis. In one particular embodiment, systemic sclerosis (SSc) is treated or prevented. In another particular embodiment, scleroderma is treated or prevented. In another particular embodiment, myelofibrosis (MF) is treated or prevented.

Systemic Sclerosis (SSc, also called scleroderma) is a severely debilitating fibrotic disease. TGFβ is a potent profibrotic cytokine that has been shown to be critical for the promotion of several pathological processes including increased collagen deposition in skin and lungs in SSc (Varga, J. and Abraham, D., 2007; Varga, J. and Whitfield, M.L., 2009; Gabrielli, A. et al., 2009; Lafyatis, R., 2014; Allanore, Y. et al., 2015). SSc represents a major unmet therapeutic challenge with the life expectancy of patients with newly diagnosed SSc being approximately eleven years (Mayes, M.D. et al., 2003). A recent clinical study validated TGFβ as a driver of fibrosis in SSc human patients with the demonstration of a dramatic reversal in fibrosis following blockade of TGFβ by the neutralizing antibody fresolimumab (Rice, L.M. et al., 2015). This clinical proof-of-principal, together with extensive preclinical data demonstrating the importance of TGFβ in promoting fibrosis in SSc and other diseases, provides a compelling rationale for the use of TGFβ binding agents in accordance with the present disclosure for the treatment of SSc patients.

In myelofibrosis (MF), bone marrow fibrosis is a hallmark of the disease and its degree correlates with clinical features including anemia. Administration of a TGFβ blocking agent has been shown to result in resolution of myelofibrosis in several preclinical studies (Wang, J.C. et al., 2006; Vannucchi, A.M. et al., 2005) and supports a dual pathological role for TGFβ in MF, namely promotion of bone marrow fibrosis as well as myeloproliferation. Elevated intraplatelet, peripheral blood mononuclear cell, and megakaryocyte-associated TGFβ has been documented in MF patients. The over-expression of TGFβ in clinical samples, together with extensive preclinical data on the effect of TGFβ neutralization in MF models, provides a compelling rationale for the use of TGFβ binding agents in accordance with the present disclosure for the treatment of MF patients.

Other exemplary embodiments of fibrosis that may be prevented or treated include, for example and without limitation: interstitial lung disease; human fibrotic lung disease (e.g., obliterative bronchiolitis, idiopathic pulmonary fibrosis, pulmonary fibrosis from a known etiology, tumor stroma in lung disease, systemic sclerosis affecting lungs, Hermansky-Pudlak syndrome, coal worker’s pneumoconiosis, asbestosis, silicosis, chronic pulmonary hypertension); AIDS-associated treatable types of fibrosis, including lung fibrosis, cystic fibrosis, liver fibrosis, heart fibrosis, mediastinal fibrosis, retroperitoneal cavity fibrosis, bone marrow fibrosis, skin fibrosis; scleroderma; and systemic sclerosis. Specific forms of fibrosis that can be treated or prevented include those that affect any organ or tissue or cell of the body, such as human tenon’s fibroblasts, kidney, lung, intestine, liver, heart, bone marrow, genitalia, skin and eye. These diseases include, but are not limited to, cystic fibrosis, systemic sclerosis, chronic obstructive pulmonary disease (COPD), Dupuytren’s contracture, glomerulonephritis, liver fibrosis, post-infarction cardiac fibrosis, restenosis, ocular surgery-induced fibrosis, and scarring. Genetic disorders of connective tissue can also be treated, and include but are not limited to, Marfan syndrome (MFS) and Osteogenesis imperfecta.

In some embodiments, the polypeptide or TGFβ binding agent described herein is used for inhibiting differentiation of fibroblasts into myofibroblasts.

In some embodiments, the polypeptide or TGFβ binding agent described herein is used for treatment or prevention of a fibroproliferative disorder. Fibroproliferative disorders are characterized by proliferation of fibroblasts plus the corresponding overexpression of extracellular matrix such as fibronectin, laminin and collagen.

In some embodiments, the polypeptide or TGFβ binding agent described herein is used for treatment or prevention of cancer, including for example and without limitation, lung cancer, head and neck cancer, melanoma, colon cancer, pancreatic cancer, colorectal cancer, hepatic cancer, breast cancer, epithelial cancer, cholangiocarcinoma, solid tumors, and the like. In some embodiments, the term “prevention” with respect to cancer may include preventing invasion or metastasis of the main tumor. In some embodiments, the term “treatment” with respect to cancer may include inhibiting TGFβ-mediated suppression of the immune response in the tumor microenvironment. With respect to solid tumors, the immunosuppressive role of TGFβ in the tumor microenvironment has been clearly demonstrated preclinically. Additionally, it was recently shown in a clinical study that the lack of response to an immune checkpoint inhibitor in patients with bladder cancer is associated with TGFβ signaling in the tumor microenvironement, supporting the concept that TGFβ restrains anti-tumor immunity, and suggesting that TGFβ inhibitors may have single agent activity in some tumor settings, and act in other tumor settings to enhance anti-tumor activity when combined with immune checkpoint inhibitors.

In some embodiments, the polypeptide construct or TGFβ binding agent described herein is used for treatment or prevention of a disease of abnormal cell growth and/or dysregulated apoptosis. Examples of such diseases include, but are not limited to, cancer, mesothelioma, bladder cancer, pancreatic cancer, skin cancer, cancer of the head or neck, cutaneous or intraocular melanoma, ovarian cancer, breast cancer, uterine cancer, carcinoma of the fallopian tubes, carcinoma of the endometrium, carcinoma of the cervix, carcinoma of the vagina, carcinoma of the vulva, bone cancer, colon cancer, rectal cancer, cancer of the anal region, stomach cancer, gastrointestinal (gastric, colorectal and/or duodenal) cancer, chronic lymphocytic leukemia, acute lymphocytic leukemia, esophageal cancer, cancer of the small intestine, cancer of the endocrine system, cancer of the thyroid gland, cancer of the parathyroid gland, cancer of the adrenal gland, sarcoma of soft tissue, cancer of the urethra, cancer of the penis, testicular cancer, hepatocellular (hepatic and/or biliary duct) cancer, primary or secondary central nervous system tumor, primary or secondary brain tumor, Hodgkin’s disease, chronic or acute leukemia, chronic myeloid leukemia, lymphocytic lymphoma, lymphoblastic leukemia, follicular lymphoma, lymphoid malignancies of T-cell or B-cell origin, melanoma, multiple myeloma, oral cancer, non-small-cell lung cancer, prostate cancer, small-cell lung cancer, cancer of the kidney and/or ureter, renal cell carcinoma, carcinoma of the renal pelvis, neoplasms of the central nervous system, primary central nervous system lymphoma, non-Hodgkin’s lymphoma, spinal axis tumors, brain stem glioma, pituitary adenoma, adrenocortical cancer, gall bladder cancer, cancer of the spleen, cholangiocarcinoma, fibrosarcoma, neuroblastoma, retinoblastoma or a combination thereof.

In some embodiments, the polypeptide construct or TGFβ binding agent described herein is used for treatment or prevention of a disease or disorder selected from the group consisting of bladder cancer, brain cancer, breast cancer, bone marrow cancer, cervical cancer, chronic lymphocytic leukemia, acute lymphocytic leukemia, colorectal cancer, esophageal cancer, hepatocellular cancer, lymphoblastic leukemia, follicular lymphoma, lymphoid malignancies of T-cell or B-cell origin, melanoma, myelogenous leukemia, myeloma, oral cancer, ovarian cancer, non-small- cell lung cancer, prostate cancer, small-cell lung cancer and spleen cancer.

In some embodiments, the polypeptide construct or TGFβ binding agent described herein is used for treatment or prevention of a disease or disorder that is a hematological cancer, such as leukemia, lymphoma, or myeloma. In some embodiments, the cancer is selected from the group consisting of Hodgkin’s lymphoma, non-Hodgkin’s lymphoma (NHL), cutaneous B-cell lymphoma, activated B-cell lymphoma, diffuse large B-cell lymphoma (DLBCL), mantle cell lymphoma (MCL), follicular center lymphoma, transformed lymphoma, lymphocytic lymphoma of intermediate differentiation, intermediate lymphocytic lymphoma (ILL), diffuse poorly differentiated lymphocytic lymphoma (PDL), centrocytic lymphoma, diffuse small-cleaved cell lymphoma (DSCCL), peripheral T-cell lymphomas (PTCL), cutaneous T-Cell lymphoma, mantle zone lymphoma, low grade follicular lymphoma, multiple myeloma (MM), chronic lymphocytic leukemia (CLL), diffuse large B-cell lymphoma (DLBCL), myelodysplastic syndrome (MDS), acute T cell leukemia, acute myeloid leukemia (AML), acute promyelocytic leukemia, acute myeloblastic leukemia, acute megakaryoblastic leukemia, precursor B acute lymphoblastic leukemia, precursor T acute lymphoblastic leukemia, Burkitt’s leukemia (Burkitt’s lymphoma), acute biphenotypic leukemia, chronic myeloid lymphoma, chronic myelogenous leukemia (CML), and chronic monocytic leukemia. In a specific embodiment, the disease or disorder is myeloma. In a specific embodiment, the disease or disorder is myelodysplastic syndromes (MDS). In another specific embodiment, the disease or disorder is acute myeloid leukemia (AML). In another specific embodiment, the disease or disorder is chronic lymphocytic leukemia (CLL). In yet another specific embodiment, the myeloma is multiple myeloma (MM).

In other embodiments, the polypeptide construct or TGFβ binding agent described herein is used for treatment or prevention of a disease or disorder that is a solid tumor malignancy. In some embodiments, the solid tumor malignancy is selected from the group consisting of a carcinoma, an adenocarcinoma, an adrenocortical carcinoma, a colon adenocarcinoma, a colorectal adenocarcinoma, a colorectal carcinoma, a ductal cell carcinoma, a lung carcinoma, a thyroid carcinoma, a nasopharyngeal carcinoma, a melanoma, a non-melanoma skin carcinoma, and a lung cancer.

In some embodiments, the solid tumor malignancy is an advanced non-CNS-primary solid tumor. In some embodiments, the solid tumor malignancy is selected from a group consisting of gastric/gastroesophageal junction (GEJ) cancer, bladder/urothelial cancer, and non-small-cell lung cancer (NSCLC).

In some embodiments, the immune checkpoint inhibitor to be administered in combination with the polypeptide or TGFβ binding agent described herein can be any pharmaceutical agent that inhibits or blocks the activity of an inhibitory immune checkpoint molecule. In specific embodiments, the activity is binding to the natural binding partner of the immune checkpoint molecule. If the immune checkpoint molecule is a receptor, the activity can be ligand-binding activity. If the immune checkpoint molecule is a ligand, the activity can be receptor-binding activity.

In specific embodiments, the immune checkpoint inhibitor to be administered in combination with the polypeptide or TGFβ binding agent described herein is a negative checkpoint regulator that is involved in T-Cell activation. In certain, more specific embodiments, such a negative checkpoint regulator is Cytotoxic T-lymphocyte antigen-4 (CTLA-4), CD80, CD86, Programmed cell death 1 (PD-1), Programmed cell death ligand 1 (PD-L1), Programmed cell death ligand 2 (PD-L2), Lymphocyte activation gene-3 (LAG-3; also known as CD223), Galectin-3, B and T lymphocyte attenuator (BTLA), T-cell membrane protein 3 (TIM3), Galectin-9 (GAL9), B7-H1, B7-H3, B7-H4, T-Cell immunoreceptor with Ig and ITIM domains (TIGIT/Vstm3/WUCAM/VSIG9), V-domain Ig suppressor of T-Cell activation (VISTA), Glucocorticoid-induced tumor necrosis factor receptor-related (GITR) protein, Herpes Virus Entry Mediator (HVEM), OX40, CD27, CD28, CD137. CGEN-15001T, CGEN-15022, CGEN-15027, CGEN-15049, CGEN-15052, or CGEN-15092. An overview such checkpoint regulators and drugs that target them is set forth in Table 1. In a specific embodiment, the immune checkpoint inhibitor is an inhibitor of PD-1, PD-L1, PD-L2, CTLA-4, LAG3, TIM-3, VISTA, A2AR, B7-H3, B7-H4, BTLA, IDO, or TDO.

In certain embodiments, the immune checkpoint inhibitor can be an antibody, a small molecule, or an oligonucleotide (such as an aptamer, an shRNA, miRNA, siRNA, or antisense DNA). In specific embodiments, the immune checkpoint inhibitor has been approved by Food and Drug Administration (FDA) in the United States or a foreign counterpart agency for the treatment of the cancer or a disease caused by the pathogen.

In specific embodiments, the immune checkpoint inhibitor is an antibody that binds to and inhibits the activity of the immune checkpoint. Antibodies that can be the immune checkpoint inhibitor include, but are not limited to, monoclonal antibodies (including Fc-optimized monoclonal antibodies), polyclonal antibodies, multispecific antibodies (e.g., bispecific antibodies), antibody fragments retaining antigen-binding activity, such as Fv, Fab, Fab′, F(ab′)2, diabodies, linear antibodies, single-chain antibody molecules (e.g., scFv), multispecific antibodies formed from antibody fragments, and fusion proteins containing antibody fragments. In a specific embodiment, the antibody is a monoclonal antibody. Preferably, the antibody is a humanized antibody.

In certain embodiments, the immune checkpoint inhibitor is an inhibitor of PD-1. In a specific embodiment, the immune checkpoint inhibitor is a monoclonal antibody that binds to and inhibits the activity (e.g., ligand-binding activity) of PD-1.

In certain embodiments, the monoclonal antibody is selected from the group consisting of nivolumab, pidilizumab, MEDI0680, pembrolizumab, AMP-224, AMP-514, STI-A1110, TSR-042, AUR-012, cemiplimab, spartalizumab, camrelizumab, sintilimab, tislelizumab, and toripalimab.

In a specific embodiment, the monoclonal antibody is nivolumab, pidilizumab, MEDI0680, or pembrolizumab. In a further specific embodiment, the monoclonal antibody is nivolumab. In another specific embodiment, the immune checkpoint inhibitor that is an inhibitor of PD-1 is AMP-224. In another specific embodiment, the immune checkpoint inhibitor that is an inhibitor of PD-1 is pidilizumab. In another specific embodiment, the immune checkpoint inhibitor that is an inhibitor of PD-1 is pembrolizumab. In another specific embodiment, the immune checkpoint inhibitor that is an inhibitor of PD-1 is MEDI0680. In another specific embodiment, the immune checkpoint inhibitor that is an inhibitor of PD-1 is STI-A1110. In another specific embodiment, the immune checkpoint inhibitor that is an inhibitor of PD-1 is TSR-042. In another specific embodiment, the immune checkpoint inhibitor that is an inhibitor of PD-1 is AUR-012.

In certain embodiments, the immune checkpoint inhibitor is an inhibitor of PD-L1. In a specific embodiment, the immune checkpoint inhibitor is a monoclonal antibody that binds to and inhibits the activity (e.g., receptor-binding activity) of PD-L1.

In certain embodiments, the immune checkpoint inhibitor is selected from the group consisting of mpdl3280A, durvalumab, avelumab, BMS-936559, atezolizumab, RG7446, and STI-A1010.

In a specific embodiment, the monoclonal antibody is mpdl3280A, durvalumab, avelumab, BMS-936559, or atezolizumab. In another specific embodiment, the immune checkpoint inhibitor that is an inhibitor of PD-L1 is RG7446. In another specific embodiment, the immune checkpoint inhibitor that is an inhibitor of PD-L1 is STI-A1010.

In certain embodiments, the immune checkpoint inhibitor is an inhibitor of CTLA4 (for example, ipilimumab).

In certain embodiments, the immune checkpoint inhibitor is an inhibitor of LAG3 (for example, BMS-986016).

In certain embodiments, immune checkpoint inhibitors to be administered in combination with the polypeptide or TGFβ binding agent described herein include but are not limited to: OPDIVO® (nivolumab); YERVOY® (ipilimumab); relatilimab; linrodostat; EMPLICITI® (elotuzumab); BMS-986258; BMS 986315; BMS-986207; BMS-986249; and BMS-986218.

PD-1 inhibitors useful in the combinations described herein include any molecule capable of inhibiting, blocking, abrogating or interfering with the activity or expression of PD-1. In particular, an anti-PD-1inhibitor can be a small molecule compound, a nucleic acid, a polypeptide, an antibody, a peptibody, a diabody, a minibody, a single-domain antibody or nanobody, a single-chain variable fragment (ScFv), or a functional fragment or variant thereof. In one instance the PD-1 inhibitor is a small molecule compound (e.g., a compound having a molecule weight of less than about 1000 Da.) In other embodiments, useful PD-1 inhibitors in the combinations described herein include nucleic acids and polypeptides.

In some embodiments, there are provided methods for preventing or inhibiting recurrence of a cancer after treatment, e.g., after drug treatment or surgical excision. In some embodiments, there are provided methods for delaying the progression of a cancer, wherein cancer re-growth is delayed by more than 30%, or by more than 50%, or by more than 70%, and/or wherein the survival periods of affected subjects are increased. There are further provided methods for enhancing the efficacy of cancer therapies for the treatment of cancer, selected from the group comprising resection, chemotherapy, radiation therapy, immunotherapy, and/or gene therapy, comprising administering a polypeptide or TGFβ binding agent as described herein, and simultaneously, separately or sequentially administrating said cancer therapy. The term “enhancing the efficacy of a cancer therapy”, as used herein, refers to an improvement of conventional cancer treatments and includes reduction of the amount of the anti-cancer composition which is applied during the conventional cancer treatment, e.g. amount of radiation in radiotherapy, of chemotherapeutics in chemotherapy, of immunotherapeutics in immunotherapy or of vectors in gene based therapies, and/or to an increase in efficacy of the conventional therapy and the anti-cancer composition when applied at conventional doses or amounts during the conventional cancer therapy. In one embodiment, enhancing the efficacy of a cancer therapy refers to prolonging the survival rate of subjects receiving the therapy.

In some embodiments, the polypeptide or TGFβ binding agent described herein is used for treating or preventing bone marrow failure in a subject, e.g., a human having or at risk of developing bone marrow failure. Exemplary types of bone marrow failure include, without limitation, SDS (also known as Shwachman-Bodian-Diamond syndrome or SBDS), Fanconi anemia (FA), dyskeratosis congenita (DC), congenital amegakaryocytic thrombocytopenia (CAMT), Blackfan-Diamond anemia (BDA), and reticular dysgenesis (RD). Shwachman-Diamond Syndrome (SDS) patients suffer from bone marrow failure, exocrine pancreatic dysfunction, skeletal anomalies, and increased risk of acute myeloid leukemia. In a particular embodiment, the polypeptide or TGFβ binding agent described herein is used for treating or preventing Fanconi anemia (FA) in a subject. In another particular embodiment, the polypeptide or TGFβ binding agent described herein is used for treating or preventing Shwachman-Diamond Syndrome (SDS) in a subject.

Fanconi anemia (FA) is the most common inherited bone marrow failure syndrome. FA patients develop bone marrow failure during the first decade of life due to attrition of hematopoietic stem and progenitor cells (HSPCs). FA is caused by mutations in one of nineteen Fanconi anemia complementation group (FANC) genes, the products of which cooperate in the FA/BRCA DNA repair pathway. Bone marrow failure in FA may be the result, directly or indirectly, of hyperactivation of growth-suppressive pathways induced, in part, by genotoxic stress. Canonical TGFβ pathway-mediated growth suppression of hematopoietic stem cells (HSCs) was recently identified as a cause of bone marrow failure in FA (Rio, P. and Bueren, J.A., 2016; Zhang, H. et al., 2016). Shwachman-Diamond Syndrome (SDS) is another rare bone marrow failure syndrome that is caused by mutations in the SBDS gene (Boocock, G.R. et al., 2003; Rogers, Z.R., 2018). It has been shown that the TGFβ pathway is dysregulated in SDS cells. Taken together, such findings provide a compelling rationale for the use of TGFβ binding agents in accordance with the present disclosure for the treatment of bone marrow failure syndromes.

In some embodiments, therefore, there is provided a method for treating or preventing bone marrow failure such as SDS, the method comprising administering an effective amount of a polypeptide or TGFβ binding agent in accordance with the present disclosure to a subject in need thereof. In such embodiments, the polypeptide or TGFβ binding agent may reduce or inhibit a symptom or sequelae associated with SDS. Exemplary symptoms or sequelae associated with SDS are selected from the group consisting of neutropenia (e.g., exhibiting an absolute neutrophil count <1500/mL), anemia, thrombocytopenia (e.g., exhibiting a platelet count below 50,000/mm³), exocrine pancreatic dysfunction, growth retardation, chronic steatorrhea, metaphyseal dysplasia, myelodysplasia, megakaryocyte dysplasia, erythroid dysplasia, acute myeloid leukemia (AML), and generalized osteopenia. See, e.g., WO2016/138300 and WO2019/018662, for more discussion of bone marrow failure.

As used herein, the terms “effective amount” and “therapeutically effective amount” are used interchangeably to refer to the amount or dose of a compound or composition, upon single or multiple dose administration to a subject, which provides the desired effect (e.g., the desired biological or medicinal response, e.g., to ameliorate, lessen or prevent a disease, disorder or condition) in the subject being treated. In some embodiments, an effective amount is an amount or dose of a compound or composition that prevents or treats a TGFβ-associated disease or condition in a subject, as described herein. In some embodiments, an effective amount is an amount or dose of a compound or composition that inhibits one or more activity of TGFβ (e.g., TGFβ1 and/or TGFβ3) in a subject, as described herein.

The term “inhibition” or “inhibiting” is used herein to refer generally to reducing, slowing, restricting, delaying, suppressing, blocking, neutralizing, hindering, or preventing a process, such as without limitation reducing or slowing growth, spread or survival of a TGFβ-associated disease or condition, such as without limitation, fibrosis, a cancer or tumor, or a bone marrow failure.

The term “treating” or “treatment” for purposes of this disclosure refers to both therapeutic treatment and prophylactic or preventative measures, wherein the object is to ameliorate the targeted disease or condition. Those in need of treatment include those already with the disorder as well as those prone to have the disorder or those in whom the disorder is to be prevented. In certain embodiments “treating” or “treatment” refers to ameliorating at least one physical parameter, such as skin thickening, fibrotic scarring, or tumor size, growth, or migration. In certain embodiments, “treating” or “treatment” refers to inhibiting or improving a disease or condition, either physically (e.g., stabilization of a discernible symptom), physiologically (e.g., stabilization of a physical parameter), or both. In certain embodiments, “treating” or “treatment” refers to delaying the onset (or recurrence) of a disease or condition. The term “treating” or “treatment” may refer to any indicia of success in the treatment or amelioration of a disease or condition, including any objective or subjective parameter such as abatement; remission; diminishing of symptoms or making the disease or condition more tolerable to the subject; improving a subject’s physical or mental well-being, such as reducing pain or discomfort experienced by the patient; and, in some situations additionally improving at least one clinical parameter of a disease or condition.

In some embodiments of the present disclosure, “treating” refers to neutralizing the biologic activity of excess TGFβ. It may be determined by suitable clinical variables of improvement; by pathologic evaluation of the effects on e.g. fibrosis and/or immunosuppression or prevention of fibrosis; by a direct inhibition of TGFβ signaling; or by another measure suitable for the disease or condition being treated.

As used herein, “preventing” or “prevention” is intended to refer at least to the reduction of the likelihood of, or the risk of, or susceptibility to acquiring a disease or disorder (i.e., causing at least one of the clinical symptoms of the disease not to develop in a patient that may be exposed to or predisposed to or at risk of the disease but does not yet experience or display symptoms of the disease). The term “prevention” or “preventing” is also used to describe the administration of a compound or composition described herein to a subject who is at risk of (or susceptible to) such a disease or condition. Subjects amenable to treatment for prevention of a disease or condition include individuals at risk of the disease or condition but not showing symptoms, as well as patients presently showing symptoms. In some embodiments, “prevention” or “preventing” is used to describe the administration of a compound or composition described herein to a subject who has been diagnosed with or treated for a disease or condition and is at risk of recurrence of the disease or condition.

In some embodiments, treatment or prevention are within the context of the present invention if there is a measurable difference between the performances of subjects treated using the TGFβ binding agents, compositions and methods provided herein as compared to members of a placebo group, historical control, or between subsequent tests given to the same subject.

The term “subject” includes living organisms with a TGFβ-associated disease or condition, or who are susceptible to or at risk thereof. Examples of subjects include mammals, e.g., humans, monkeys, cows, rabbits, sheep, goats, pigs, dogs, cats, rats, mice, and transgenic species thereof. The term “subject” generally includes animals susceptible to states characterized by TGFβ-associated diseases or conditions such as fibrosis or cancer, e.g., mammals, e.g. primates, e.g. humans. The animal can also be an animal model for a disorder, e.g., a mouse model, a xenograft recipient, and the like. In certain embodiments, the subject is a human.

There are no particular limitations on the dose of each of TGFβ binding agents for use in compositions provided herein. Exemplary doses include milligram or microgram amounts of the compound per kilogram of subject or sample weight (e.g., about 50 micrograms per kilogram to about 500 milligrams per kilogram, about 1 milligram per kilogram to about 100 milligrams per kilogram, about 1 milligram per kilogram to about 50 milligram per kilogram, about 1 milligram per kilogram to about 10 milligrams per kilogram, or about 3 milligrams per kilogram to about 5 milligrams per kilogram). Additional exemplary doses include doses of about 5 to about 500 mg, about 25 to about 300 mg, about 25 to about 200 mg, about 50 to about 150 mg, or about 50, about 100, about 150 mg, about 200 mg or about 250 mg, and, for example, daily or twice daily, or lower or higher amounts.

In some embodiments, the dose range for adult humans is generally from 0.005 mg to 10 g/day. Polypeptides, TGFβ binding agents, and compositions thereof may be provided in Unit dosage form, e.g., in a unit which is effective at such dosage or as a multiple of the same, for instance, units containing 5 mg to 500 mg, usually around 10 mg to 200 mg. A dosage unit can include from, for example, 1 to 30 mg, 1 to 40 mg, 1 to 100 mg, 1 to 300 mg, 1 to 500 mg, 2 to 500 mg, 3 to 100 mg, 5 to 20 mg, 5 to 100 mg (e.g. 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 150 mg, 200 mg, 250 mg, 300 mg, 350 mg, 400 mg, 450 mg, or 500 mg) of a polypeptide, TGFβ binding agent, or composition described herein.

It should be understood that the effective amount of polypeptide or TGFβ binding agent for therapeutic treatment of a disease or condition varies depending upon the manner of administration, the age, body weight, and general health of the subject. Ultimately, the attending physician or veterinarian decides the appropriate amount and dosage regimen. It should be understood that the dosage or amount of a polypeptide or TGFβ binding agent used, alone or in combination with one or more active compounds to be administered, depends on the individual case and is, as is customary, to be adapted to the individual circumstances to achieve an optimum effect. Dosing and administration regimens are within the purview of the skilled artisan, and appropriate doses depend upon a number of factors within the knowledge of the ordinarily skilled physician, veterinarian, or researcher (e.g., see Wells et al. eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000)). For example, dosing and administration regimens depend on the nature and the severity of the disorder to be treated, and also on the sex, age, weight and individual responsiveness of the human or animal to be treated, on the efficacy and duration of action of the compounds used, on whether the therapy is acute or chronic or prophylactic, and/or on whether other active compounds are administered in addition to the therapeutic molecule(s).

Administration of compounds and compositions provided herein can be carried out using known procedures, at dosages and for periods of time effective to achieved the desired purpose. Dosage regimens can be adjusted to provide the optimum therapeutic response. For example, several divided doses may be administered daily or the dose may be proportionally reduced as indicated by the exigencies of the therapeutic situation. In some embodiments, a compound or composition is administered at an effective dosage sufficient to prevent or treat fibrosis in a subject.

There are no particular limitations on the route of administration of each of TGFβ binding agents for use in compositions provided herein. A polypeptide, TGFβ binding agent or composition thereof may be administered using any suitable route or means, such as without limitation via oral, parenteral, intravenous, intraperitoneal, intramuscular, subcutaneous, sublingual, topical, or nasal administration, via inhalation, via injection, via infusion, or via such other routes as are known in the art. In a particular embodiment, the polypeptide, TGFβ binding agent or composition thereof is administered by injection or infusion, for example and without limitation, intravenously, intraperitoneally, intramuscularly, or subcutaneously.

In some embodiments, in accordance with the methods of the present disclosure, one or more symptom of development or progression of a TGFβ-associated disease or condition is reduced by at least 5%, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% in a subject.

In some embodiments, in accordance with the methods of the present disclosure, fibrotic symptoms are reduced in a subject. For example, the polypeptide, TGFβ binding agent or composition may reduce fibrosis, fibrotic scarring, or skin thickening in a subject by at least 5%, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%.

In some embodiments, in accordance with the methods of the present disclosure, the differentiation of fibroblasts into myofibroblasts is inhibited in a subject, e.g., by at least 5%, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%.

In some embodiments, in accordance with the methods of the present disclosure, tumor growth and/or metastasis is inhibited in a subject, e.g., by at least 5%, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%.

In some embodiments, in accordance with the methods of the present disclosure, hematopoietic colony formation and/or hematopoiesis in bone marrow hematopoietic stem or progenitor cells (HSPCs) is increased in the bone marrow of a subject, e.g., by at least 5%, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%.

In some embodiments, in accordance with the methods of the present disclosure, a positive response in lung fibrosis is revealed as a consistent slowing in the rate of decline in lung function, as measured by forced vital capacity.

In some embodiments, in accordance with the methods of the present disclosure, a positive response for skin fibrosis associated with systemic sclerosis is determined by an improvement in the Modified Rodnan Skin Score (MRSS). For example, the MRSS may improve in a subject by at least 5%, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%.

In some embodiments, in bone marrow failure diseases including myelofibrosis, positive responses are revealed by improvements in anemia (for example, transfusion-independent patients exhibiting an increase in hemoglobin level, transfusion dependent patients become transfusion independent).

In some embodiments, the polypeptide or TGFβ binding agent may be conjugated with a therapeutic moiety, as described herein. A desirable therapeutic moiety may be chosen for its ability to prevent or treat the same disease or condition being targeted by the polypeptide or TGFβ binding agent.

In some embodiments, there are provided methods for prevention or treatment of a TGFβ-associated disease or condition in a subject by administering an effective amount of a polypeptide or TGFβ binding agent described herein, such that the TGFβ-associated disease or condition is prevented or treated in the subject.

In some embodiments, there are provided methods of inhibiting TGFβ in a subject by administering an effective amount of a polypeptide or TGFβ binding agent described herein, such that TGFβ is inhibited in the subject. In some such embodiments, there are provided methods of inhibiting TGFβ3 in a subject by administering an effective amount of a polypeptide or TGFβ binding agent described herein, such that TGFβ3 is inhibited in the subject. In some such embodiments, there are provided methods of inhibiting TGFβ3 and TGFβ1 in a subject by administering an effective amount of a polypeptide or TGFβ binding agent described herein, such that TGFβ3 and TGFβ3 are inhibited in the subject.

In some embodiments, there are provided methods for inhibiting differentiation of fibroblasts into myofibroblasts either in vitro, ex vivo, or in vivo.

In some embodiments of therapeutic and prophylactic treatments provided herein, the polypeptide or TGFβ binding agent is administered in combination with one or more additional therapy or therapeutic agent. The additional therapy or therapeutic agent can be administered before, after or simultaneously with the administration of the polypeptide, TGFβ binding agent or composition described herein. In some embodiments, the additional therapy or therapeutic agent is formulated together with the polypeptide or TGFβ binding agent in the same composition. In other embodiments, the additional therapy or therapeutic agent is administered separately. Examples of additional therapies and therapeutic agents include, without limitation, an anti-fibrotic agent; an anti-cancer agent; another TGFβ-binding agent or inhibitor, such as an antibody, antibody fragment, antigen-binding fragment, soluble TGFβ ligand trap, and the like. In one embodiment, the additional therapeutic agent is nintedanib (marketed under the brand names Ofev® and Vargatef®). In one embodiment, the additional therapeutic agent is pirfenidone. In one embodiment, the additional therapeutic agent is an immune checkpoint inhibitor.

Alternatively, in some embodiments, the polypeptide or TGFβ binding agent may be conjugated with a detectable moiety or a diagnostic moiety that is useful for tracking the polypeptide or TGFβ binding agent, or cells or tissues expressing TGFβ. In some such embodiments, there are provided methods of diagnosis of a TGFβ-associated disease or condition comprising administering to a subject a polypeptide or TGFβ binding agent of the present disclosure conjugated with a detectable moiety or a diagnostic moiety, and detecting the polypeptide or TGFβ binding agent such that a disease or condition associated with TGFβ (e.g., overexpression of TGFβ1 and/or TGFβ3) is diagnosed.

### Kits

In accordance with the present disclosure, polypeptides, TGFβ binding agents and pharmaceutical compositions described herein may be assembled into kits or pharmaceutical systems for use in treating or preventing TGFβ-associated diseases or conditions. Kits or pharmaceutical systems may comprise a container (e.g. packaging, a box, a carton, a vial, etc.), having in close confinement therein one or more container, such as vials, tubes, ampoules, bottles, and the like, that contains the polypeptide, TGFβ binding agent or pharmaceutical composition. Additional kit components may include acids, bases, buffering agents, inorganic salts, solvents, antioxidants, preservatives, or metal chelators. The additional kit components may be present as pure compositions, or as aqueous or organic solutions that incorporate one or more additional kit components. Any or all of the kit components optionally further comprise buffers. Kits may also include tools for administration, such as needles, syringes, and the like. The kit may be used according to the methods described herein and may include instructions for use in such methods. Kits may also include instructions for administration and use of the polypeptide, TGFβ binding agent or pharmaceutical composition.

Sequences are given in the Listing of Sequences in Table 2. In Table 2, the N-terminal IDR in the human TGFβRII ectodomain and sequences derived therefrom are shown underlined; the C-terminal IDR in the human TGFβRII ectodomain and sequences derived therefrom are shown double underlined; Gly—Ser linker regions are shown in italics and underlined; and multimerization domains are shown in italics and boldface.

TABLE 2

Listing of Sequences.

| | | | |
|---|---|---|---|
| SEQ ID NO: | Descriptio n | Sequence | #amino acids |
| 1 | hTGFβRII-ECD | IPPHVQKSVNNDMIVTDNNGAVKFPQLCKFCDVRFSTCDNQKS CMSNCSITSICEKPQEVCVAVWRKNDENITLETVCHDPKLPYH DFILEDAASPKCIMKEKKKPGETFFMCSCSSDECNDNIIFSEE YNTSNPD | 136 |
| 2 | TGFβR-LBD | PQLCKFCDVRFSTCDNQKSCMSNCSITSICEKPQEVCVAVWRK NDENITLETVCHDPKLPYHDFILEDAASPKCIMKEKKKPGETF FMCSCSSDECNDNIIF | 102 |
| 3 | N-terminal IDR | IPPHVQKSVNNDMIVTDNNGAVKF | 24 |
| 4 | C-terminal IDR | SEEYNTSNPD | 10 |
| 5 | TGFβ-binding region from T22d35-Fc-IgG1-v1(CC) | IPPHVQKSVNNDMIVTDNNGAVKFPQLCKFCDVRFSTCDNQKS CMSNCSITSICEKPQEVCVAVWRKNDENITLETVCHDPKLPYH DFILEDAASPKCIMKEKKKPGETFFMCSCSSDECNDNIIFSEE YNTSNPDIPPHVQKSVNNDMIVTDNNGAVKFPQLCKFCDVRFS TCDNQKSCMSNCSITSICEKPQEVCVAVWRKNDENITLETVCH DPKLPYHDFILEDAASPKCIMKEKKKPGETFEMCSCSSDECND NIIFSEEYNTSNPD | 272 |
| 6 | T22d35-Fc-IgG1-v1(CC) (Control) | IPPHVQKSVNNDMIVTDNNGAVKFPQLCKFCDVRFSTCDNQKS CMSNCSITSICEKPQEVCVAVWRKNDENITLETVCHDPKLPYH DFILEDAASPKCIMKEKKKPGETFFMCSCSSDECNDNIIFSEE YNTSNPDIPPHVQKSVNNDMIVTDNNGAVKFPQLCKFCDVRFS TCDNQKSCMSNCSITSICEKPQEVCVAVWRKNDENITLETVCH DPKLPYHDFILEDAASPKCIMKEKKKPGETFFMCSCSSDECND NIIFSEEYNTSNPDTHTCPPCPAPELLGGPSVFLFPPKPKDTL MISRTPEVTCVVVDVSHEDPEVKFNWYVDGVEVHNAKTKPREE QYNSTYRVVSVLTVLHQDWLNGKEYKCKVSNKALPAPIEKTIS KAKGQPREPQVYTLPPSRDELTKNQVSLTCLVKGFYPSDIAVE WESNGQPENNYKTTPPVLDSDGSFFLYSKLTVDKSRWQQGNVF SCSVMHEALHNHYTQKSLSLSPG | 496 |
| 7 | Linker | SEEYNTSNPDIPPHVQKSVNNDMIVTDNNGAVKF | 34 |
| 8 | Linker | SEEYNTSNPDDNNGAVKF | 18 |
| 9 | Linker | SEEYNTSNPDNGAVKF | 16 |
| 10 | Linker | SEEYNTSNPDNDMIVTDNNGAVKF | 24 |
| 11 | Linker | SEEYNTIPPHVQKSVNNDMIVTDNNGAVKF | 30 |
| 12 | Linker | SEEYNDMIVTDNNGAVKF | 18 |
| 13 | Linker | SEEYPHVQKSVNNDMIVTDNNGAVKF | 26 |
| 14 | Linker | SEEYNTSNPDVTDNNGAVKF | 20 |
| 15 | Linker | SEEYMIVTDNNGAVKF | 16 |
| 16 | Linker | SEEYNTSNPDPHVQKSVNNDMIVTDNNGAVKF | 32 |
| 17 | Linker | GGGGSGGGGSGGGGSGGGGSGGGGSGGGGS | 30 |
| 18 | Linker | SEEYNTGGGGSGGGGSGGGGSGGGGSGGGG | 30 |
| 19 | Linker | SEEYNTSNPDVQKSVNNDMIVTDNNGAVKF | 30 |
| 20 | Linker | GGGGSGGGGSGGGGSG | 16 |
| 21 | Linker | GGGGSGGGGSGGGGSGGG | 18 |
| 22 | Linker | SEEYDMIVTDNNGAVKF | 17 |
| 23 | Linker | SEEYNNDMIVTDNNGAVKF | 19 |
| 24 | Linker | GGGGSGGGGSGGGGSGGGGSGGGGSGGGGSGGGG | 34 |
| 25 | Linker | SEEYNTSNPDGGGGSGGGGSGGGGSGGGGSGGGG | 34 |
| 26 | Linker | SEEYNTSNPDVNNDMIVTDNNGAVKF | 26 |
| 27 | TGFβ-binding region -Protein 61 | IPPHVQKSVNNDMIVTDNNGAVKFPQLCKFCDVRFSTCDNQKS CMSNCSITSICEKPQEVCVAVWRKNDENITLETVCHDPKLPYH DFILEDAASPKCIMKEKKKPGETFFMCSCSSDECNDNIIFSEE YNTSNPDDNNGAVKFPQLCKFCDVRFSTCDNQKSCMSNCSITS ICEKPQEVCVAVWRKNDENITLETVCHDPKLPYHDFILEDAAS PKCIMKEKKKPGETFFMCSCSSDECNDNIIFSEEYNTSNPDNG AVKF | 262 |
| 28 | TGFβ-binding region -Protein 93 | IPPHVQKSVNNDMIVTDNNGAVKFPQLCKFCDVRFSTCDNQKS CMSNCSITSICEKPQEVCVAVWRKNDENITLETVCHDPKLPYH DFILEDAASPKCIMKEKKKPGETFFMCSCSSDECNDNIIFSEE YNTSNPDNDMIVTDNNGAVKFPQLCKFCDVRFSTCDNQKSCMS NCSITSICEKPQEVCVAVWRKNDENITLETVCHDPKLPYHDFI LEDAASPKCIMKEKKKPGETFFMCSCSSDECNDNIIFSEEYNT SNPDNGAVKF | 268 |
| 29 | TGFβ-binding region -Protein 96 | IPPHVQKSVNNDMIVTDNNGAVKFPQLCKFCDVRFSTCDNQKS CMSNCSITSICEKPQEVCVAVWRKNDENITLETVCHDPKLPYH DFILEDAASPKCIMKEKKKPGETFFMCSCSSDECNDNIIFSEE YNTSNPDDNNGAVKFPQLCKFCDVRFSTCDNQKSCMSNCSITS ICEKPQEVCVAVWRKNDENITLETVCHDPKLPYHDFILEDAAS PKCIMKEKKKPGETFFMCSCSSDECNDNIIFSEEYNTSNPDNG AVKF | 262 |
| 30 | TGFβ-binding region -Protein 97 | IPPHVQKSVNNDMIVTDNNGAVKFPQLCKFCDVRFSTCDNQKS CMSNCSITSICEKPQEVCVAVWRKNDENITLETVCHDPKLPYH DFILEDAASPKCIMKEKKKPGETFFMCSCSSDECNDNIIFSEE YNTSNPDNDMIVTDNNGAVKFPQLCKFCDVRFSTCDNQKSCMS NCSITSICEKPQEVCVAVWRKNDENITLETVCHDPKLPYHDFI LEDAASPKCIMKEKKKPGETFFMCSCSSDECNDNIIFSEEYNT SNPD | 262 |
| 31 | TGFβ-binding region -Protein 99 | IPPHVQKSVNNDMIVTDNNGAVKFPQLCKFCDVRFSTCDNQKS CMSNCSITSICEKPQEVCVAVWRKNDENITLETVCHDPKLPYH DFILEDAASPKCIMKEKKKPGETFFMCSCSSDECNDNIIFSEE YNTIPPHVQKSVNNDMIVTDNNGAVKFPQLCKFCDVRFSTCDN QKSCMSNCSITSICEKPQEVCVAVWRKNDENITLETVCHDPKL PYHDFILEDAASPKCIMKEKKKPGETFFMCSCSSDECNDNIIF SEEYNTIPPHVQKSVNNDMIVTDNNGAVKF | 288 |
| 32 | TGFβ-binding region -Protein 101 | IPPHVQKSVNNDMIVTDNNGAVKFPQLCKFCDVRFSTCDNQKS CMSNCSITSICEKPQEVCVAVWRKNDENITLETVCHDPKLPYH DFILEDAASPKCIMKEKKKPGETFFMCSCSSDECNDNIIFSEE YNDMIVTDNNGAVKFPQLCKFCDVRFSTCDNQKSCMSNCSITS ICEKPQEVCVAVWRKNDENITLETVCHDPKLPYHDFILEDAAS PKCIMKEKKKPGETFFMCSCSSDECNDNIIFSEEYNTIPPHVQ KSVNNDMIVTDNNGAVKF | 276 |
| 33 | TGFβ-binding region -Protein 106 | IPPHVQKSVNNDMIVTDNNGAVKFPQLCKFCDVRFSTCDNQKS CMSNCSITSICEKPQEVCVAVWRKNDENITLETVCHDPKLPYH DFILEDAASPKCIMKEKKKPGETFFMCSCSSDECNDNIIFSEE YPHVQKSVNNDMIVTDNNGAVKFPQLCKFCDVRFSTCDNQKSC | 280 |
| | | MSNCSITSICEKPQEVCVAVWRKNDENITLETVCHDPKLPYHD FILEDAASPKCIMKEKKKPGETFFMCSCSSDECNDNIIFSEEY PHVQKSVNNDMIVTDNNGAVKF | |
| 34 | TGFβ-binding region -Protein 107 | IPPHVQKSVNNDMIVTDNNGAVKFPQLCKFCDVRFSTCDNQKS CMSNCSITSICEKPQEVCVAVWRKNDENITLETVCHDPKLPYH DFILEDAASPKCIMKEKKKPGETFFMCSCSSDECNDNIIFSEE YNTSNPDDNNGAVKFPQLCKFCDVRFSTCDNQKSCMSNCSITS ICEKPQEVCVAVWRKNDENITLETVCHDPKLPYHDFILEDAAS PKCIMKEKKKPGETFFMCSCSSDECNDNIIFSEEYNTSNPD | 256 |
| 35 | TGFβ-binding region -Protein 108 | IPPHVQKSVNNDMIVTDNNGAVKFPQLCKFCDVRFSTCDNQKS CMSNCSITSICEKPQEVCVAVWRKNDENITLETVCHDPKLPYH DFILEDAASPKCIMKEKKKPGETFFMCSCSSDECNDNIIFSEE YNTSNPDNGAVKFPQLCKFCDVRFSTCDNQKSCMSNCSITSIC EKPQEVCVAVWRKNDENITLETVCHDPKLPYHDFILEDAASPK CIMKEKKKPGETFFMCSCSSDECNDNIIFSEEYNTSNPDNGAV KF | 260 |
| 36 | TGFβ-binding region -Protein 109 | IPPHVQKSVNNDMIVTDNNGAVKFPQLCKFCDVRFSTCDNQKS CMSNCSITSICEKPQEVCVAVWRKNDENITLETVCHDPKLPYH DFILEDAASPKCIMKEKKKPGETFEMCSCSSDECNDNIIFSEE YNTSNPDVTDNNGAVKFPQLCKFCDVRFSTCDNQKSCMSNCSI TSICEKPQEVCVAVWRKNDENITLETVCHDPKLPYHDFILEDA ASPKCIMKEKKKPGETFFMCSCSSDECNDNIIFSEEYNTSNPD NGAVKF | 264 |
| 37 | TGFβ-binding region -Protein 112 | IPPHVQKSVNNDMIVTDNNGAVKFPQLCKFCDVRFSTCDNQKS CMSNCSITSICEKPQEVCVAVWRKNDENITLETVCHDPKLPYH DFILEDAASPKCIMKEKKKPGETFFMCSCSSDECNDNIIFSEE YNDMIVTDNNGAVKFPQLCKFCDVRFSTCDNQKSCMSNCSITS ICEKPQEVCVAVWRKNDENITLETVCHDPKLPYHDFILEDAAS PKCIMKEKKKPGETFFMCSCSSDECNDNIIFSEEYMIVTDNNG AVKF | 262 |
| 38 | TGFβ-binding region -Protein 113 | IPPHVQKSVNNDMIVTDNNGAVKFPQLCKFCDVRFSTCDNQKS CMSNCSITSICEKPQEVCVAVWRKNDENITLETVCHDPKLPYH DFILEDAASPKCIMKEKKKPGETFFMCSCSSDECNDNIIFSEE YNTSNPDPHVQKSVNNDMIVTDNNGAVKFPQLCKFCDVRFSTC DNQKSCMSNCSITSICEKPQEVCVAVWRKNDENITLETVCHDP KLPYHDFILEDAASPKCIMKEKKKPGETFFMCSCSSDECNDNI IFSEEYNTSNPDPHVQKSVNNDMIVTDNNGAVKF | 292 |
| 39 | TGFβ-binding region -Protein 115 | IPPHVQKSVNNDMIVTDNNGAVKFPQLCKFCDVRFSTCDNQKS CMSNCSITSICEKPQEVCVAVWRKNDENITLETVCHDPKLPYH DFILEDAASPKCIMKEKKKPGETFFMCSCSSDECNDNIIFSEE YNTSNPDPHVQKSVNNDMIVTDNNGAVKFPQLCKFCDVRFSTC DNQKSCMSNCSITSICEKPQEVCVAVWRKNDENITLETVCHDP KLPYHDFILEDAASPKCIMKEKKKPGETFFMCSCSSDECNDNI IFSEEYNTSNPDPHVQKSVNNDMIVTDNNGAVKF | 292 |
| 40 | TGFβ-binding region -Protein 116 | IPPHVQKSVNNDMIVTDNNGAVKFPQLCKFCDVRFSTCDNQKS CMSNCSITSICEKPQEVCVAVWRKNDENITLETVCHDPKLPYH DFILEDAASPKCIMKEKKKPGETFFMCSCSSDECNDNIIFSEE YNTSNPDPHVQKSVNNDMIVTDNNGAVKFPQLCKFCDVRFSTC DNQKSCMSNCSITSICEKPQEVCVAVWRKNDENITLETVCHDP KLPYHDFILEDAASPKCIMKEKKKPGETFFMCSCSSDECNDNI IFSEEYNTSNPDIPPHVQKSVNNDMIVTDNNGAVKF | 294 |
| 41 | TGFβ-binding region -Protein 128 | IPPHVQKSVNNDMIVTDNNGAVKFPQLCKFCDVRFSTCDNQKS CMSNCSITSICEKPQEVCVAVWRKNDENITLETVCHDPKLPYH DFILEDAASPKCIMKEKKKPGETFFMCSCSSDECNDNIIFSEE YNDMIVTDNNGAVKFPQLCKFCDVRFSTCDNQKSCMSNCSITS ICEKPQEVCVAVWRKNDENITLETVCHDPKLPYHDFILEDAAS PKCIMKEKKKPGETFFMCSCSSDECNDNIIFSEEYNTIPPHVQ KSVNNDMIVTDNNGAVKF | 276 |
| 42 | TGFβ-binding region -Protein 112 | IPPHVQKSVNNDMIVTDNNGAVKFPQLCKFCDVRFSTCDNQKS CMSNCSITSICEKPQEVCVAVWRKNDENITLETVCHDPKLPYH DFILEDAASPKCIMKEKKKPGETFFMCSCSSDECNDNIIFSEE YNDMIVTDNNGAVKFPQLCKFCDVRFSTCDNQKSCMSNCSITS ICEKPQEVCVAVWRKNDENITLETVCHDPKLPYHDFILEDAAS PKCIMKEKKKPGETFFMCSCSSDECNDNIIFGGGGSGGGGSGG GGSGGGGSGGGGSGGGGS | 276 |
| 43 | TGFβ-binding region -Protein 130 | IPPHVQKSVNNDMIVTDNNGAVKFPQLCKFCDVRFSTCDNQKS CMSNCSITSICEKPQEVCVAVWRKNDENITLETVCHDPKLPYH DFILEDAASPKCIMKEKKKPGETFFMCSCSSDECNDNIIFSEE YNDMIVTDNNGAVKFPQLCKFCDVRFSTCDNQKSCMSNCSITS ICEKPQEVCVAVWRKNDENITLETVCHDPKLPYHDFILEDAAS PKCIMKEKKKPGETFFMCSCSSDECNDNIIFSEEYNTGGGGSG GGGSGGGGSGGGGSGGGG | 276 |
| 44 | TGFβ-binding region -Protein 131 | IPPHVQKSVNNDMIVTDNNGAVKFPQLCKFCDVRFSTCDNQKS CMSNCSITSICEKPQEVCVAVWRKNDENITLETVCHDPKLPYH DFILEDAASPKCIMKEKKKPGETFEMCSCSSDECNDNIIFSEE YNDMIVTDNNGAVKFPQLCKFCDVRFSTCDNQKSCMSNCSITS | 276 |
| | | ICEKPQEVCVAVWRKNDENITLETVCHDPKLPYHDFILEDAAS PKCIMKEKKKPGETFFMCSCSSDECNDNIIFSEEYNTSNPDVQ KSVNNDMIVTDNNGAVKF | |
| 45 | TGFβ-binding region -Protein 132 | IPPHVQKSVNNDMIVTDNNGAVKFPQLCKFCDVRFSTCDNQKS CMSNCSITSICEKPQEVCVAVWRKNDENITLETVCHDPKLPYH DFILEDAASPKCIMKEKKKPGETFFMCSCSSDECNDNIIFSEE YNTSNPDDNNGAVKFPQLCKFCDVRFSTCDNQKSCMSNCSITS ICEKPQEVCVAVWRKNDENITLETVCHDPKLPYHDFILEDAAS PKCIMKEKKKPGETFFMCSCSSDECNDNIIFSEEYNTSNPDVQ KSVNNDMIVTDNNGAVKF | 276 |
| 46 | TGFβ-binding region -Protein 133 | IPPHVQKSVNNDMIVTDNNGAVKFPQLCKFCDVRFSTCDNQKS CMSNCSITSICEKPQEVCVAVWRKNDENITLETVCHDPKLPYH DFILEDAASPKCIMKEKKKPGETFFMCSCSSDECNDNIIFSEE YNTSNPDDNNGAVKFPQLCKFCDVRFSTCDNQKSCMSNCSITS ICEKPQEVCVAVWRKNDENITLETVCHDPKLPYHDFILEDAAS PKCIMKEKKKPGETFFMCSCSSDECNDNIIFSEEYNTIPPHVQ KSVNNDMIVTDNNGAVKF | 276 |
| 47 | TGFβ-binding region -Protein 134 | IPPHVQKSVNNDMIVTDNNGAVKFPQLCKFCDVRFSTCDNQKS CMSNCSITSICEKPQEVCVAVWRKNDENITLETVCHDPKLPYH DFILEDAASPKCIMKEKKKPGETFFMCSCSSDECNDNIIFSEE YNTSNPDDNNGAVKFPQLCKFCDVRFSTCDNQKSCMSNCSITS ICEKPQEVCVAVWRKNDENITLETVCHDPKLPYHDFILEDAAS PKCIMKEKKKPGETFFMCSCSSDECNDNIIFGGGGSGGGGSGG GGSG | 262 |
| 48 | TGFβ-binding region -Protein 135 | IPPHVQKSVNNDMIVTDNNGAVKFPQLCKFCDVRFSTCDNQKS CMSNCSITSICEKPQEVCVAVWRKNDENITLETVCHDPKLPYH DFILEDAASPKCIMKEKKKPGETFFMCSCSSDECNDNIIFGGG GSGGGGSGGGGSGGGPQLCKFCDVRFSTCDNQKSCMSNCSITS ICEKPQEVCVAVWRKNDENITLETVCHDPKLPYHDFILEDAAS PKCIMKEKKKPGETFFMCSCSSDECNDNIIFSEEYNTSNPDNG AVKF | 262 |
| 49 | hIgG1Fc(C C) region | THTCPPCPAPELLGGPSVFLFPPKPKDTLMISRTPEVTCVVD VSHEDPEVKFNWYVDGVEVHNAKTKPREEQYNSTYRVVSVLTV LHQDWLNGKEYKCKVSNKALPAPIEKTISKAKGQPREPQVYTL PPSRDELTKNQVSLTCLVKGFYFSDIAVEWESNGQPENNYKTT PPVLDSDGSFFLYSKLTVDKSRWQQGNVFSCSVMHEALHNHYT QKSLSLSPG | 224 |
| 50 | hIgG4Fc region | GPPCPPCPAPEFLGGPSVFLFPPKPKDTLMISRTPEVTCVVVD VSQEDPEVQFNWYVDGVEVENAKTKPREEQFNSTYRVVSVLTV LHQDWLNGKEYKCKVSNKGLPSSIEKTISKAKGQPREPQVYTL PPSQEEMTKNQVSLTCLVKGFYPSDIAVEWESNGQPENNYKTT PPVLDSDGSFFLYSRLTVDKSRWQEGNVFSCSVMHEALHNHYT QKSLSLSLG | 224 |
| 51 | hIgG1Fc | APELLGGPSVFLFPPKPKDTLMISRTPEVTCVVVDVSHEDPEV KFNWYVDGVEVHNAKTKPREEQYNSTYRVVSVLTVLHQDWLNG KEYKCKVSNKALPAPIEKTISKAKGQPREPQVYTLPPSRDELT KNQVSLTCLVKGFYPSDIAVEWESNGQPENNYKTTPPVLDSDG SFFLYSKLTVDKSRWQQGNVFSCSVMHEALHNHYTQKSLSLSP GK | 217 |
| 52 | hIgG1Fc variant | APELLGGPSVFLFPPKPKDTLMISRTPEVTCVVVDVSHEDPEV KFNWYVDGVEVHNAKTKPREEQYNSTYRVVSVLTVLHQDWLNG KEYKCKVSNKALPAPIEKTISKAKGQPREPQVYTLPPSRDELT KNQVSLTCLVKGFYPSDIAVEWESNGQPENNYKTTPPVLDSDG SFFLYSKLTVDKSRWQQGNVFSCSVMHEALHNHYTQKSLSLSP G | 216 |
| 53 | hIgG1Fc variant | PPCPAPELLGGPSVFLFPPKPKDTLMISRTPEVTCVVVDVSHE DPEVKFNWYVDGVEVENAKTKPREEQYNSTYRVVSVLTVLHQD WLNGKEYKCKVSNKALPAPIEKTISKAKGQPREPQVYTLPPSR DELTKNQVSLTCLVKGFYPSDIAVEWESNGQPENNYKTTPPVL DSDGSFFLYSKLTVDKSRWQQGNVFSCSVMHEALHNHYTQKSL SLSPG | 220 |
| 54 | hIgG1Fc variant | DKTHTCPPCPAPELLGGPSVFLFPPKPKDTLMISRTPEVTCVV VDVSHEDPEVKFNWYVDGVEVHNAKTKPREEQYNSTYRVVSVL TVLHQDWLNGKEYKCKVSNKALPAPIEKTISKAKGQPREPQVY TLPPSRDELTKNQVSLTCLVKGFYPSDIAVEWESNGQPENNYK TTPPVLDSDGSFFLYSKLTVDKSRWQQGNVFSCSVMHEALHNH YTQKSLSLSPG | 226 |
| 55 | hIgG1Fc variant | EPKSSDKTHTCPPCPAPELLGGPSVFLFPPKPKDTLMISRTPE VTCVVVDVSHEDPEVKFNWYVDGVEVHNAKTKPREEQYNSTYR VVSVLTVLHQDWLNGKEYKCKVSNKALPAPIEKTISKAKGQPR EPQVYTLPPSRDELTKNQVSLTCLVKGFYFSDIAVEWESNGQP ENNYKTTPPVLDSDGSFFLYSKLTVDKSRWQQGNVFSCSVMHE ALHNHYTQKSLSLSPG | 231 |
| 56 | hIgG1Fc variant | EPKSSDKTHTSPPSPAPELLGGPSVFLFPPKPKDTLMISRTPE VTCVVVDVSHEDPEVKFNWYVDGVEVHNAKTKPREEQYNSTYR VVSVLTVLHQDWLNGKEYKCKVSNKALPAPIEKTISKAKGQPR EPQVYTLPPSRDELTKNQVSLTCLVKGFYPSDIAVEWESNGQP | 231 |
| | | ENNYKTTPPVLDSDGSFFLYSKLTVDKSRWQQGNVFSCSVMHE ALHNHYTQKSLSLSPG | |
| 57 | hIgG2Fc | APPVAGPSVFLFPPKPKDTLMISRTPEVTCVVVDVSHEDPEVQ FNWYVDGVEVHNAKTKPREEQFNSTFRVVSVLTVVHQDWLNGK EYKCKVSNKGLPAPIEKTISKTKGQPREPQVYTLPPSREEMTK NQVSLTCLVKGFYPSDISVEWESNGQPENNYKTTPPMLDSDGS FFLYSKLTVDKSRWQQGNVFSCSVMHEALHNHYTQKSLSLSPG K | 216 |
| 58 | hIgG2Fc variant | VECPPCPAPPVAGPSVFLFPPKPKDTLMISRTPEVTCVVVDVS HEDPEVQFNWYVDGVEVHNAKTKPREEQFNSTFRVVSVLTVVH QDWLNGKEYKCKVSNKGLPAPIEKTISKTKGQPREPQVYTLPP SREEMTKNQVSLTCLVKGFYFSDIAVEWESNGQPENNYKTTPP MLDSDGSFFLYSKLTVDKSRWQQGNVFSCSVMHEALHNHYTQK SLSLSPGK | 223 |
| 59 | hIgG2Fc variant | ERKCCVECPPCPAPPVAGPSVFLFPPKPKDTLMISRTPEVTCV VVDVSHEDPEVQFNWYVDGVEVHNAKTKPREEQFNSTFRVVSV LTVVHQDWLNGKEYKCKVSNKGLPAPIEKTISKTKGQPREPQV YTLPPSREEMTKNQVSLTCLVKGFYPSDISVEWESNGQPENNY KTTPPMLDSDGSFFLYSKLTVDKSRWQQGNVFSCSVMHEALHN HYTQKSLSLSPGK | 228 |
| 60 | hIgG2Fc variant | ERKSSVECPPCPAPPVAGPSVFLFPPKPKDTLMISRTPEVTCV VVDVSHEDPEVQFNWYVDGVEVHNAKTKPREEQFNSTFRVVSV LTVVHQDWLNGKEYKCKVSNKGLPAPIEKTISKTKGQPREPQV YTLPPSREEMTKNQVSLTCLVKGFYPSDISVEWESNGQPENNY KTTPPMLDSDGSFFLYSKLTVDKSRWQQGNVFSCSVMHEALHN HYTQKSLSLSPG | 227 |
| 61 | hIgG2Fc variant | ERKSSVESPPCPAPPVAGPSVFLFPPKPKDTLMISRTPEVTCV VVDVSHEDPEVQFNWYVDGVEVHNAKTKPREEQFNSTFRVVSV LTVVHQDWLNGKEYKCKVSNKGLPAPIEKTISKTKGQPREPQV YTLPPSREEMTKNQVSLTCLVKGFYPSDISVEWESNGQPENNY KTTPPMLDSDGSFFLYSKLTVDKSRWQQGNVFSCSVMHEALHN HYTQKSLSLSPG | 227 |
| 62 | hIgG2Fc variant | ERKSSVESPPSPAPPVAGPSVFLFPPKPKDTLMISRTPEVTCV VVDVSHEDPEVQFNWYVDGVEVHNAKTKPREEQFNSTFRVVSV LTVVHQDWLNGKEYKCKVSNKGLPAPIEKTISKTKGQPREPQV YTLPPSREEMTKNQVSLTCLVKGFYPSDISVEWESNGQPENNY KTTPPMLDSDGSFFLYSKLTVDKSRWQQGNVFSCSVMHEALHN HYTQKSLSLSPG | 227 |
| 63 | hIgG2Fc variant | APPVAGPSVFLFPPKPKDTLMISRTPEVTCVVVDVSHEDPEVQ FNWYVDGVEVHNAKTKPREEQFNSTFRVVSVLTVVHQDWLNGK EYKCKVSNKGLPAPIEKTISKTKGQPREPQVYTLPPSREEMTK | 215 |
| | | NQVSLTCLVKGFYPSDISVEWESNGQPENNYKTTPPMLDSDGS FFLYSKLTVDKSRWQQGNVFSCSVMHEALHNHYTQKSLSLSPG | |
| 64 | hIgG2Fc variant | PPCPAPPVAGPSVFLFPPKPKDTLMISRTPEVTCVVVDVSHED PEVQFNWYVDGVEVHNAKTKPREEQFNSTFRVVSVLTVVHQDW LNGKEYKCKVSNKGLPAPIEKTISKTKGQPREPQVYTLPPSRE EMTKNQVSLTCLVKGFYPSDISVEWESNGQPENNYKTTPPMLD SDGSFFLYSKLTVDKSRWQQGNVFSCSVMHEALHNHYTQKSLS LSPG | 219 |
| 65 | hIgG2Fc variant | VECPPCPAPPVAGPSVFLFPPKPKDTLMISRTPEVTCVVVDVS HEDPEVQFNWYVDGVEVHNAKTKPREEQFNSTFRVVSVLTVVH QDWLNGKEYKCKVSNKGLPAPIEKTISKTKGQPREPQVYTLPP SREEMTKNQVSLTCLVKGFYPSDISVEWESNGQPENNYKTTPP MLDSDGSFFLYSKLTVDKSRWQQGNVFSCSVMHEALHNHYTQK SLSLSPG | 222 |
| 66 | hIgG3Fc | APELLGGPSVFLFPPKPKDTLMISRTPEVTCVVVDVSHEDPEV QFKWYVDGVEVENAKTKPREEQYNSTFRVVSVLTVLHQDWLNG KEYKCKVSNKALPAPIEKTISKTKGQPREPQVYTLPPSREEMT KNQVSLTCLVKGFYPSDIAVEWESSGQPENNYNTTPPMLDSDG SFFLYSKLTVDKSRWQQGNIFSCSVMHEALHNRFTQKSLSLSP GK | 217 |
| 67 | hIgG3Fc variant | APELLGGPSVFLFPPKPKDTLMISRTPEVTCVVVDVSHEDPEV QFKWYVDGVEVENAKTKPREEQYNSTFRVVSVLTVLHQDWLNG KEYKCKVSNKALPAPIEKTISKTKGQPREPQVYTLPPSREEMT KNQVSLTCLVKGFYPSDIAVEWESSGQPENNYNTTPPMLDSDG SFFLYSKLTVDKSRWQQGNIFSCSVMHEALHNRFTQKSLSLSP G | 216 |
| 68 | hIgG3Fc variant | PRCPAPELLGGPSVFLFPPKPKDTLMISRTPEVTCVVVDVSHE DPEVQFKWYVDGVEVENAKTKPREEQYNSTFRVVSVLTVLHQD WLNGKEYKCKVSNKALPAPIEKTISKTKGQPREPQVYTLPPSR EEMTKNQVSLTCLVKGFYPSDIAVEWESSGQPENNYNTTPPML DSDGSFFLYSKLTVDKSRWQQGNIFSCSVMHEALHNRFTQKSL SLSPG | 220 |
| 69 | hIgG3Fc variant | DTPPPCPRCPAPELLGGPSVFLFPPKPKDTLMISRTPEVTCVV VDVSHEDPEVQFKWYVDGVEVHNAKTKPREEQYNSTFRVVSVL TVLHQDWLNGKEYKCKVSNKALPAPIEKTISKTKGQPREPQVY TLPPSREEMTKNQVSLTCLVKGFYPSDIAVEWESSGQPENNYN TTPPMLDSDGSFFLYSKLTVDKSRWQQGNIFSCSVMHEALHNR FTQKSLSLSPG | 226 |
| 70 | hIgG3Fc variant | EPKSSDTPPPCPRCPAPELLGGPSVFLFPPKPKDTLMISRTPE VTCVVVDVSHEDPEVQFKWYVDGVEVHNAKTKPREEQYNSTFR VVSVLTVLHQDWLNGKEYKCKVSNKALPAPIEKTISKTKGQPR | 231 |
| | | EPQVYTLPPSREEMTKNQVSLTCLVKGFYPSDIAVEWESSGQP ENNYNTTPPMLDSDGSFFLYSKLTVDKSRWQQGNIFSCSVMHE ALHNRFTQKSLSLSPG | |
| 71 | hIgG3Fc variant | EPKSSDTPPPSPRCPAPELLGGPSVFLFPPKPKDTLMISRTPE VTCVVVDVSHEDPEVQFKWYVDGVEVHNAKTKPREEQYNSTFR VVSVLTVLHQDWLNGKEYKCKVSNKALPAPIEKTISKTKGQPR EPQVYTLPPSREEMTKNQVSLTCLVKGFYPSDIAVEWESSGQP ENNYNTTPPMLDSDGSFFLYSKLTVDKSRWQQGNIFSCSVMHE ALHNRFTQKSLSLSPG | 231 |
| 72 | hIgG3Fc variant | EPKSSDTPPPSPRSPAPELLGGPSVFLFPPKPKDTLMISRTPE VTCVVVDVSHEDPEVQFKWYVDGVEVHNAKTKPREEQYNSTFR VVSVLTVLHQDWLNGKEYKCKVSNKALPAPIEKTISKTKGQPR EPQVYTLPPSREEMTKNQVSLTCLVKGFYPSDIAVEWESSGQP ENNYNTTPPMLDSDGSFFLYSKLTVDKSRWQQGNIFSCSVMHE ALHNRFTQKSLSLSPG | 231 |
| 73 | hIgG4Fc | APEFLGGPSVFLFPPKPKDTLMISRTPEVTCVVVDVSQEDPEV QFNWYVDGVEVHNAKTKPREEQFNSTYRVVSVLTVLHQDWLNG KEYKCKVSNKGLPSSIEKTISKAKGQPREPQVYTLPPSQEEMT KNQVSLTCLVKGFYPSDIAVEWESNGQPENNYKTTPPVLDSDG SFFLYSRLTVDKSRWQEGNVFSCSVMHEALHNHYTQKSLSLSL GK | 217 |
| 74 | hIgG4Fc variant | APEFLGGPSVFLFPPKPKDTLMISRTPEVTCVVVDVSQEDPEV QFNWYVDGVEVHNAKTKPREEQFNSTYRVVSVLTVLHQDWLNG KEYKCKVSNKGLPSSIEKTISKAKGQPREPQVYTLPPSQEEMT KNQVSLTCLVKGFYPSDIAVEWESNGQPENNYKTTPPVLDSDG SFFLYSRLTVDKSRWQEGNVFSCSVMHEALHNHYTQKSLSLSL G | 216 |
| 75 | hIgG4Fc variant | PSCPAPEFLGGPSVFLFPPKPKDTLMISRTPEVTCVVVDVSQE DPEVQFNWYVDGVEVENAKTKPREEQFNSTYRVVSVLTVLHQD WLNGKEYKCKVSNKGLPSSIEKTISKAKGQPREPQVYTLPPSQ EEMTKNQVSLTCLVKGFYFSDIAVEWESNGQPENNYKTTPPVL DSDGSFFLYSRLTVDKSRWQEGNVFSCSVMHEALHNHYTQKSL SLSLG | 220 |
| 76 | hIgG4Fc variant | ESKYGPPCPSCPAPEFLGGPSVFLFPPKPKDTLMISRTPEVTC VVVDVSQEDPEVQFNWYVDGVEVHNAKTKPREEQFNSTYRVVS VLTVLHQDWLNGKEYKCKVSNKGLPSSIEKTISKAKGQPREPQ VYTLPPSQEEMTKNQVSLTCLVKGFYFSDIAVEWESNGQPENN YKTTPPVLDSDGSFFLYSRLTVDKSRWQEGNVFSCSVMHEALH NHYTQKSLSLSLG | 228 |
| 77 | hIgG4Fc variant | ESKYGPPCPPCPAPEFLGGPSVFLFPPKPKDTLMISRTPEVTC VVVDVSQEDPEVQFNWYVDGVEVHNAKTKPREEQFNSTYRVVS | 228 |
| | | VLTVLHQDWLNGKEYKCKVSNKGLPSSIEKTISKAKGQPREPQ VYTLPPSQEEMTKNQVSLTCLVKGFYPSDIAVEWESNGQPENN YKTTPPVLDSDGSFFLYSRLTVDKSRWQEGNVFSCSVMHEALH NHYTQKSLSLSLG | |
| 78 | hIgG4Fc variant | ESKYGPPCPPCPAPEFLGGPSVFLFPPKPKDTLMISRTPEVTC VVVDVSQEDPEVQFNWYVDGVEVHNAKTKPREEQFNSTYRVVS VLTVLHQDWLNGKEYKCKVSNKGLPSSIEKTISKAKGQPREPQ VYTLPPSQEEMTKNQVSLTCLVKGFYPSDIAVEWESNGQPENN YKTTPPVLDSDGSFFLYSKLTVDKSRWQEGNVFSCSVMHEALH NHYTQKSLSLSLG | 228 |
| 79 | hIgG4Fc variant | ESKYGPPSPSCPAPEFLGGPSVFLFPPKPKDTLMISRTPEVTC VVVDVSQEDPEVQFNWYVDGVEVHNAKTKPREEQFNSTYRVVS VLTVLHQDWLNGKEYKCKVSNKGLPSSIEKTISKAKGQPREPQ VYTLPPSQEEMTKNQVSLTCLVKGFYFSDIAVEWESNGQPENN YKTTPPVLDSDGSFFLYSRLTVDKSRWQEGNVFSCSVMHEALH NHYTQKSLSLSLG | 228 |
| 80 | hIgG4Fc variant | ESKYGPPSPSSPAPEFLGGPSVFLFPPKPKDTLMISRTPEVTC VVVDVSQEDPEVQFNWYVDGVEVHNAKTKPREEQFNSTYRVVS VLTVLHQDWLNGKEYKCKVSNKGLPSSIEKTISKAKGQPREPQ VYTLPPSQEEMTKNQVSLTCLVKGFYFSDIAVEWESNGQPENN YKTTPPVLDSDGSFFLYSRLTVDKSRWQEGNVFSCSVMHEALH NHYTQKSLSLSLG | 228 |
| 81 | Protein 61 | IPPHVQKSVNNDMIVTDNNGAVKFPQLCKFCDVRFSTCDNQKS CMSNCSITSICEKPQEVCVAVWRKNDENITLETVCHDPKLPYH DFILEDAASPKCIMKEKKKPGETFFMCSCSSDECNDNIIFSEE YNTSNPDDNNGAVKFPQLCKFCDVRFSTCDNQKSCMSNCSITS ICEKPQEVCVAVWRKNDENITLETVCHDPKLPYHDFILEDAAS PKCIMKEKKKPGETFFMCSCSSDECNDNIIFSEEYNTSNPDNG AVKFGPPCPPCPAPEFLGGPSVFLFPPKPKDTLMISRTPEVTC VVVDVSQEDPEVQFNWYVDGVEVHNAKTKPREEQFNSTYRVVS VLTVLHQDWLNGKEYKCKVSNKGLPSSIEKTISKAKGQPREPQ VYTLPPSQEEMTKNQVSLTCLVKGFYFSDIAVEWESNGQPENN YKTTPPVLDSDGSFFLYSRLTVDKSRWQEGNVFSCSVMHEALH NHYTQKSLSLSLG | 486 |
| 105 | Protein 70 | IPPHVQKSVNNDMIVTDNNGAVKFPQLCKFCDVRFSTCDNQKS CMSNCSITSICEKPQEVCVAVWRKNDENITLETVCHDPKLPYH DFILEDAASPKCIMKEKKKPGETFFMCSCSSDECNDNIIFSEE YNTSNPDNDMIVTDNNGAVKFPQLCKFCDVRFSTCDNQKSCMS NCSITSICEKPQEVCVAVWRKNDENITLETVCHDPKLPYHDFI LEDAASPKCIMKEKKKPGETFFMCSCSSDECNDNIIFSEEYNT SNPDGPPCPPCPAPEFLGGPSVFLFPPKPKDTLMISRTPEVTC VVVDVSQEDPEVQFNWYVDGVEVHNAKTKPREEQFNSTYRVVS | 486 |
| | | VLTVLHQDWLNGKEYKCKVSNKGLPSSIEKTISKAKGQPREPQ VYTLPPSQEEMTKNQVSLTCLVKGFYPSDIAVEWESNGQPENN YKTTPPVLDSDGSFFLYSRLTVDKSRWQEGNVFSCSVMHEALH NHYTQKSLSLSLG | |
| 82 | Protein 93 | IPPHVQKSVNNDMIVTDNNGAVKFPQLCKFCDVRFSTCDNQKS CMSNCSITSICEKPQEVCVAVWRKNDENITLETVCHDPKLPYH DFILEDAASPKCIMKEKKKPGETFFMCSCSSDECNDNIIFSEE YNTSNPDNDMIVTDNNGAVKFPQLCKFCDVRFSTCDNQKSCMS NCSITSICEKPQEVCVAVWRKNDENITLETVCHDPKLPYHDFI LEDAASPKCIMKEKKKPGETFFMCSCSSDECNDNIIFSEEYNT SNPDNGAVKFGPPCPPCPAPEFLGGPSVFLFPPKPKDTLMISR TPEVTCVVVDVSQEDPEVQFNWYVDGVEVHNAKTKPREEQFNS TYRVVSVLTVLHQDWLNGKEYKCKVSNKGLPSSIEKTISKAKG QPREPQVYTLPPSQEEMTKNQVSLTCLVKGFYPSDIAVEWESN GQPENNYKTTPPVLDSDGSFFLYSRLTVDKSRWQEGNVFSCSV MHEALHNHYTQKSLSLSLG | 492 |
| 83 | Protein 94 | IPPHVQKSVNNDMIVTDNNGAVKFPQLCKFCDVRFSTCDNQKS CMSNCSITSICEKPQEVCVAVWRKNDENITLETVCHDPKLPYH DFILEDAASPKCIMKEKKKPGETFFMCSCSSDECNDNIIFSEE YNTSNPDNDMIVTDNNGAVKFPQLCKFCDVRFSTCDNQKSCMS NCSITSICEKPQEVCVAVWRKNDENITLETVCHDPKLPYHDFI LEDAASPKCIMKEKKKPGETFFMCSCSSDECNDNIIFSEEYNT SNPDNDMIVTDNNGAVKFGPPCPPCPAPEFLGGPSVFLFPPKP KDTLMISRTPEVTCVVVDVSQEDPEVQFNWYVDGVEVHNAKTK PREEQFNSTYRVVSVLTVLHQDWLNGKEYKCKVSNKGLPSSIE KTISKAKGQPREPQVYTLPPSQEEMTKNQVSLTCLVKGFYPSD IAVEWESNGQPENNYKTTPPVLDSDGSFFLYSRLTVDKSRWQE GNVFSCSVMHEALHNHYTQKSLSLSLG | 500 |
| 84 | Protein 96 | IPPHVQKSVNNDMIVTDNNGAVKFPQLCKFCDVRFSTCDNQKS CMSNCSITSICEKPQEVCVAVWRKNDENITLETVCHDPKLPYH DFILEDAASPKCIMKEKKKPGETFFMCSCSSDECNDNIIFSEE YNTSNPDDNNGAVKFPQLCKFCDVRFSTCDNQKSCMSNCSITS ICEKPQEVCVAVWRKNDENITLETVCHDPKLPYHDFILEDAAS PKCIMKEKKKPGETFFMCSCSSDECNDNIIFSEEYNTSNPDNG AVKFTHTCPPCPAPELLGGPSVFLFPPKPKDTLMISRTPEVTC VVVDVSHEDPEVKFNWYVDGVEVHNAKTKPREEQYNSTYRVVS VLTVLHQDWLNGKEYKCKVSNKALPAPIEKTISKAKGQPREPQ VYTLPPSRDELTKNQVSLTCLVKGFYPSDIAVEWESNGQPENN YKTTPPVLDSDGSFFLYSKLTVDKSRWQQGNVFSCSVMHEALH NHYTQKSLSLSPG | 486 |
| 85 | Protein 97 | IPPHVQKSVNNDMIVTDNNGAVKFPQLCKFCDVRFSTCDNQKS CMSNCSITSICEKPQEVCVAVWRKNDENITLETVCHDPKLPYH DFILEDAASPKCIMKEKKKPGETFFMCSCSSDECNDNIIFSEE YNTSNPDNDMIVTDNNGAVKFPQLCKFCDVRFSTCDNQKSCMS | 486 |
| | | NCSITSICEKPQEVCVAVWRKNDENITLETVCHDPKLPYHDFI LEDAASPKCIMKEKKKPGETFFMCSCSSDECNDNIIFSEEYNT SNPDTHTCPPCPAPELLGGPSVFLFPPKPKDTLMISRTPEVTC VVVDVSHEDPEVKFNWYVDGVEVHNAKTKPREEQYNSTYRVVS VLTVLHQDWLNGKEYKCKVSNKALPAPIEKTISKAKGQPREPQ VYTLPPSRDELTKNQVSLTCLVKGFYPSDIAVEWESNGQPENN YKTTPPVLDSDGSFFLYSKLTVDKSRWQQGNVFSCSVMHEALH NHYTQKSLSLSPG | |
| 86 | Protein 99 | IPPHVQKSVNNDMIVTDNNGAVKFPQLCKFCDVRFSTCDNQKS CMSNCSITSICEKPQEVCVAVWRKNDENITLETVCHDPKLPYH DFILEDAASPKCIMKEKKKPGETFFMCSCSSDECNDNIIFSEE YNTIPPHVQKSVNNDMIVTDNNGAVKFPQLCKFCDVRFSTCDN QKSCMSNCSITSICEKPQEVCVAVWRKNDENITLETVCHDPKL PYHDFILEDAASPKCIMKEKKKPGETFFMCSCSSDECNDNIIF SEEYNTIPPHVQKSVNNDMIVTDNNGAVKFGPPCPPCPAPEFL GGPSVFLFPPKPKDTLMISRTPEVTCVVVDVSQEDPEVQFNWY VDGVEVENAKTKPREEQFNSTYRVVSVLTVLHQDWLNGKEYKC KVSNKGLPSSIEKTISKAKGQPREPQVYTLPPSQEEMTKNQVS LTCLVKGFYPSDIAVEWESNGQPENNYKTTPPVLDSDGSFFLY SRLTVDKSRWQEGNVFSCSVMHEALHNHYTQKSLSLSLG | 512 |
| 87 | Protein 101 | IPPHVQKSVNNDMIVTDNNGAVKFPQLCKFCDVRFSTCDNQKS CMSNCSITSICEKPQEVCVAVWRKNDENITLETVCHDPKLPYH DFILEDAASPKCIMKEKKKPGETFFMCSCSSDECNDNIIFSEE YNDMIVTDNNGAVKFPQLCKFCDVRFSTCDNQKSCMSNCSITS ICEKPQEVCVAVWRKNDENITLETVCHDPKLPYHDFILEDAAS PKCIMKEKKKPGETFFMCSCSSDECNDNIIFSEEYNTIPPHVQ KSVNNDMIVTDNNGAVKFTHTCPPCPAPELLGGPSVFLFPPKP KDTLMISRTPEVTCVVVDVSHEDPEVKFNWYVDGVEVHNAKTK PREEQYNSTYRVVSVLTVLHQDWLNGKEYKCKVSNKALPAPIE KTISKAKGQPREPQVYTLPPSRDELTKNQVSLTCLVKGFYPSD IAVEWESNGQPENNYKTTPPVLDSDGSFFLYSKLTVDKSRWQQ GNVFSCSVMHEALHNHYTQKSLSLSPG | 500 |
| 88 | Protein 106 | IPPHVQKSVNNDMIVTDNNGAVKFPQLCKFCDVRFSTCDNQKS CMSNCSITSICEKPQEVCVAVWRKNDENITLETVCHDPKLPYH DFILEDAASPKCIMKEKKKPGETFFMCSCSSDECNDNIIFSEE YPHVQKSVNNDMIVTDNNGAVKFPQLCKFCDVRFSTCDNQKSC MSNCSITSICEKPQEVCVAVWRKNDENITLETVCHDPKLPYHD FILEDAASPKCIMKEKKKPGETFFMCSCSSDECNDNIIFSEEY PHVQKSVNNDMIVTDNNGAVKFTHTCPPCPAPELLGGPSVFLF PPKPKDTLMISRTPEVTCVVVDVSHEDPEVKFNWYVDGVEVHN AKTKPREEQYNSTYRVVSVLTVLHQDWLNGKEYKCKVSNKALP APIEKTISKAKGQPREPQVYTLPPSRDELTKNQVSLTCLVKGF YPSDIAVEWESNGQPENNYKTTPPVLDSDGSFFLYSKLTVDKS RWQQGNVFSCSVMHEALHNHYTQKSLSLSPG | 504 |
| 89 | Protein 107 | IPPHVQKSVNNDMIVTDNNGAVKFPQLCKFCDVRFSTCDNQKS CMSNCSITSICEKPQEVCVAVWRKNDENITLETVCHDPKLPYH DFILEDAASPKCIMKEKKKPGETFFMCSCSSDECNDNIIFSEE YNTSNPDDNNGAVKFPQLCKFCDVRFSTCDNQKSCMSNCSITS ICEKPQEVCVAVWRKNDENITLETVCHDPKLPYHDFILEDAAS PKCIMKEKKKPGETFFMCSCSSDECNDNIIFSEEYNTSNPDTH TCPPCPAPELLGGPSVFLFPPKPKDTLMISRTPEVTCVVVDVS HEDPEVKFNWYVDGVEVENAKTKPREEQYNSTYRVVSVLTVLH QDWLNGKEYKCKVSNKALPAPIEKTISKAKGQPREPQVYTLPP SRDELTKNQVSLTCLVKGFYPSDIAVEWESNGQPENNYKTTPP VLDSDGSFFLYSKLTVDKSRWQQGNVFSCSVMHEALHNHYTQK SLSLSPG | 480 |
| 90 | Protein 108 | IPPHVQKSVNNDMIVTDNNGAVKFPQLCKFCDVRFSTCDNQKS CMSNCSITSICEKPQEVCVAVWRKNDENITLETVCHDPKLPYH DFILEDAASPKCIMKEKKKPGETFFMCSCSSDECNDNIIFSEE YNTSNPDNGAVKFPQLCKFCDVRFSTCDNQKSCMSNCSITSIC EKPQEVCVAVWRKNDENITLETVCHDPKLPYHDFILEDAASPK CIMKEKKKPGETFFMCSCSSDECNDNIIFSEEYNTSNPDNGAV KFTHTCPPCPAPELLGGPSVFLFPPKPKDTLMISRTPEVTCVV VDVSHEDPEVKFNWYVDGVEVHNAKTKPREEQYNSTYRVVSVL TVLHQDWLNGKEYKCKVSNKALPAPIEKTISKAKGQPREPQVY TLPPSRDELTKNQVSLTCLVKGFYFSDIAVEWESNGQPENNYK TTPPVLDSDGSFFLYSKLTVDKSRWQQGNVFSCSVMHEALHNH YTQKSLSLSPG | 484 |
| 91 | Protein 109 | IPPHVQKSVNNDMIVTDNNGAVKFPQLCKFCDVRFSTCDNQKS CMSNCSITSICEKPQEVCVAVWRKNDENITLETVCHDPKLPYH DFILEDAASPKCIMKEKKKPGETFFMCSCSSDECNDNIIFSEE YNTSNPDVTDNNGAVKFPQLCKFCDVRFSTCDNQKSCMSNCSI TSICEKPQEVCVAVWRKNDENITLETVCHDPKLPYHDFILEDA ASPKCIMKEKKKPGETFFMCSCSSDECNDNIIFSEEYNTSNPD NGAVKFTHTCPPCPAPELLGGPSVFLFPPKPKDTLMISRTPEV TCVVVDVSHEDPEVKFNWYVDGVEVHNAKTKPREEQYNSTYRV VSVLTVLHQDWLNGKEYKCKVSNKALPAPIEKTISKAKGQPRE PQVYTLPPSRDELTKNQVSLTCLVKGFYPSDIAVEWESNGQPE NNYKTTPPVLDSDGSFFLYSKLTVDKSRWQQGNVFSCSVMHEA LHNHYTQKSLSLSPG | 488 |
| 92 | Protein 112 | IPPHVQKSVNNDMIVTDNNGAVKFPQLCKFCDVRFSTCDNQKS CMSNCSITSICEKPQEVCVAVWRKNDENITLETVCHDPKLPYH DFILEDAASPKCIMKEKKKPGETFEMCSCSSDECNDNIIFSEE YNDMIVTDNNGAVKFPQLCKFCDVRFSTCDNQKSCMSNCSITS | 486 |
| | | ICEKPQEVCVAVWRKNDENITLETVCHDPKLPYHDFILEDAAS PKCIMKEKKKPGETFFMCSCSSDECNDNIIFSEEYMIVTDNNG AVKFTHTCPPCPAPELLGGPSVFLFPPKPKDTLMISRTPEVTC VVVDVSHEDPEVKFNWYVDGVEVHNAKTKPREEQYNSTYRVVS VLTVLHQDWLNGKEYKCKVSNKALPAPIEKTISKAKGQPREPQ VYTLPPSRDELTKNQVSLTCLVKGFYPSDIAVEWESNGQPENN YKTTPPVLDSDGSFFLYSKLTVDKSRWQQGNVFSCSVMHEALH NHYTQKSLSLSPG | |
| 93 | Protein 113 | IPPHVQKSVNNDMIVTDNNGAVKFPQLCKFCDVRFSTCDNQKS CMSNCSITSICEKPQEVCVAVWRKNDENITLETVCHDPKLPYH DFILEDAASPKCIMKEKKKPGETFFMCSCSSDECNDNIIFSEE YNTSNPDPHVQKSVNNDMIVTDNNGAVKFPQLCKFCDVRFSTC DNQKSCMSNCSITSICEKPQEVCVAVWRKNDENITLETVCHDP KLPYHDFILEDAASPKCIMKEKKKPGETFFMCSCSSDECNDNI IFSEEYNTSNPDPHVQKSVNNDMIVTDNNGAVKFTHTCPPCPA PELLGGPSVFLFPPKPKDTLMISRTPEVTCVVVDVSHEDPEVK FNWYVDGVEVENAKTKPREEQYNSTYRVVSVLTVLHQDWLNGK EYKCKVSNKALPAPIEKTISKAKGQPREPQVYTLPPSRDELTK NQVSLTCLVKGFYPSDIAVEWESNGQPENNYKTTPPVLDSDGS FFLYSKLTVDKSRWQQGNVFSCSVMHEALHNHYTQKSLSLSPG | 516 |
| 94 | Protein 115 | IPPHVQKSVNNDMIVTDNNGAVKFPQLCKFCDVRFSTCDNQKS CMSNCSITSICEKPQEVCVAVWRKNDENITLETVCHDPKLPYH DFILEDAASPKCIMKEKKKPGETFFMCSCSSDECNDNIIFSEE YNTSNPDPHVQKSVNNDMIVTDNNGAVKFPQLCKFCDVRFSTC DNQKSCMSNCSITSICEKPQEVCVAVWRKNDENITLETVCHDP KLPYHDFILEDAASPKCIMKEKKKPGETFFMCSCSSDECNDNI IFSEEYNTSNPDPHVQKSVNNDMIVTDNNGAVKFGPPCPPCPA PEFLGGPSVFLFPPKPKDTLMISRTPEVTCVVVDVSQEDPEVQ FNWYVDGVEVENAKTKPREEQFNSTYRVVSVLTVLHQDWLNGK EYKCKVSNKGLPSSIEKTISKAKGQPREPQVYTLPPSQEEMTK NQVSLTCLVKGFYPSDIAVEWESNGQPENNYKTTPPVLDSDGS FFLYSRLTVDKSRWQEGNVFSCSVMHEALHNHYTQKSLSLSLG | 516 |
| 95 | Protein 116 | IPPHVQKSVNNDMIVTDNNGAVKFPQLCKFCDVRFSTCDNQKS CMSNCSITSICEKPQEVCVAVWRKNDENITLETVCHDPKLPYH DFILEDAASPKCIMKEKKKPGETFFMCSCSSDECNDNIIFSEE YNTSNPDPHVQKSVNNDMIVTDNNGAVKFPQLCKFCDVRFSTC DNQKSCMSNCSITSICEKPQEVCVAVWRKNDENITLETVCHDP KLPYHDFILEDAASPKCIMKEKKKPGETFFMCSCSSDECNDNI IFSEEYNTSNPDIPPHVQKSVNNDMIVTDNNGAVKFTHTCPPC PAPELLGGPSVFLFPPKPKDTLMISRTPEVTCVVVDVSHEDPE VKFNWYVDGVEVHNAKTKPREEQYNSTYRVVSVLTVLHQDWLN | 518 |
| | | GKEYKCKVSNKALPAPIEKTISKAKGQPREPQVYTLPPSRDEL TKNQVSLTCLVKGFYPSDIAVEWESNGQPENNYKTTPPVLDSD GSFFLYSKLTVDKSRWQQGNVFSCSVMHEALHNHYTQKSLSLS PG | |
| 96 | Protein 128 | IPPHVQKSVNNDMIVTDNNGAVKFPQLCKFCDVRFSTCDNQKS CMSNCSITSICEKPQEVCVAVWRKNDENITLETVCHDPKLPYH DFILEDAASPKCIMKEKKKPGETFFMCSCSSDECNDNIIFSEE YNDMIVTDNNGAVKFPQLCKFCDVRFSTCDNQKSCMSNCSITS ICEKPQEVCVAVWRKNDENITLETVCHDPKLPYHDFILEDAAS PKCIMKEKKKPGETFFMCSCSSDECNDNIIFSEEYNTIPPHVQ KSVNNDMIVTDNNGAVKFGPPCPPCPAPEFLGGPSVFLFPPKP KDTLMISRTPEVTCVVVDVSQEDPEVQFNWYVDGVEVHNAKTK PREEQFNSTYRVVSVLTVLHQDWLNGKEYKCKVSNKGLPSSIE KTISKAKGQPREPQVYTLPPSQEEMTKNQVSLTCLVKGFYPSD IAVEWESNGQPENNYKTTPPVLDSDGSFFLYSRLTVDKSRWQE GNVFSCSVMHEALHNHYTQKSLSLSLG | 500 |
| 97 | Protein 129 | IPPHVQKSVNNDMIVTDNNGAVKFPQLCKFCDVRFSTCDNQKS CMSNCSITSICEKPQEVCVAVWRKNDENITLETVCHDPKLPYH DFILEDAASPKCIMKEKKKPGETFFMCSCSSDECNDNIIFSEE YNDMIVTDNNGAVKFPQLCKFCDVRFSTCDNQKSCMSNCSITS ICEKPQEVCVAVWRKNDENITLETVCHDPKLPYHDFILEDAAS PKCIMKEKKKPGETFFMCSCSSDECNDNIIFGGGGSGGGGSGG GGSGGGGSGGGGSGGGGSTHTCPPCPAPELLGGPSVFLFPPKP KDTLMISRTPEVTCVVVDVSHEDPEVKFNWYVDGVEVHNAKTK PREEQYNSTYRVVSVLTVLHQDWLNGKEYKCKVSNKALPAPIE KTISKAKGQPREPQVYTLPPSRDELTKNQVSLTCLVKGFYPSD IAVEWESNGQPENNYKTTPPVLDSDGSFFLYSKLTVDKSRWQQ GNVFSCSVMHEALHNHYTQKSLSLSPG | 500 |
| 98 | Protein 130 | IPPHVQKSVNNDMIVTDNNGAVKFPQLCKFCDVRFSTCDNQKS CMSNCSITSICEKPQEVCVAVWRKNDENITLETVCHDPKLPYH DFILEDAASPKCIMKEKKKPGETFFMCSCSSDECNDNIIFSEE YNDMIVTDNNGAVKFPQLCKFCDVRFSTCDNQKSCMSNCSITS ICEKPQEVCVAVWRKNDENITLETVCHDPKLPYHDFILEDAAS PKCIMKEKKKPGETFFMCSCSSDECNDNIIFSEEYNTGGGGSG GGGSGGGGSGGGGSGGGGTHTCPPCPAPELLGGPSVFLFPPKP KDTLMISRTPEVTCVVVDVSHEDPEVKFNWYVDGVEVHNAKTK PREEQYNSTYRVVSVLTVLHQDWLNGKEYKCKVSNKALPAPIE KTISKAKGQPREPQVYTLPPSRDELTKNQVSLTCLVKGFYPSD IAVEWESNGQPENNYKTTPPVLDSDGSFFLYSKLTVDKSRWQQ GNVFSCSVMHEALHNHYTQKSLSLSPG | 500 |
| 99 | Protein 131 | IPPHVQKSVNNDMIVTDNNGAVKFPQLCKFCDVRFSTCDNQKS CMSNCSITSICEKPQEVCVAVWRKNDENITLETVCHDPKLPYH DFILEDAASPKCIMKEKKKPGETFFMCSCSSDECNDNIIFSEE YNDMIVTDNNGAVKFPQLCKFCDVRFSTCDNQKSCMSNCSITS ICEKPQEVCVAVWRKNDENITLETVCHDPKLPYHDFILEDAAS PKCIMKEKKKPGETFFMCSCSSDECNDNIIFSEEYNTSNPDVQ KSVNNDMIVTDNNGAVKFTHTCPPCPAPELLGGPSVFLFPPKP KDTLMISRTPEVTCVVVDVSHEDPEVKFNWYVDGVEVHNAKTK PREEQYNSTYRVVSVLTVLHQDWLNGKEYKCKVSNKALPAPIE KTISKAKGQPREPQVYTLPPSRDELTKNQVSLTCLVKGFYPSD IAVEWESNGQPENNYKTTPPVLDSDGSFFLYSKLTVDKSRWQQ GNVFSCSVMHEALHNHYTQKSLSLSPG | 500 |
| 100 | Protein 132 | IPPHVQKSVNNDMIVTDNNGAVKFPQLCKFCDVRFSTCDNQKS CMSNCSITSICEKPQEVCVAVWRKNDENITLETVCHDPKLPYH DFILEDAASPKCIMKEKKKPGETFFMCSCSSDECNDNIIFSEE YNTSNPDDNNGAVKFPQLCKFCDVRFSTCDNQKSCMSNCSITS ICEKPQEVCVAVWRKNDENITLETVCHDPKLPYHDFILEDAAS PKCIMKEKKKPGETFFMCSCSSDECNDNIIFSEEYNTSNPDVQ KSVNNDMIVTDNNGAVKFTHTCPPCPAPELLGGPSVFLFPPKP KDTLMISRTPEVTCVVVDVSHEDPEVKFNWYVDGVEVHNAKTK PREEQYNSTYRVVSVLTVLHQDWLNGKEYKCKVSNKALPAPIE KTISKAKGQPREPQVYTLPPSRDELTKNQVSLTCLVKGFYPSD IAVEWESNGQPENNYKTTPPVLDSDGSFFLYSKLTVDKSRWQQ GNVFSCSVMHEALHNHYTQKSLSLSPG | 500 |
| 101 | Protein 133 | IPPHVQKSVNNDMIVTDNNGAVKFPQLCKFCDVRFSTCDNQKS CMSNCSITSICEKPQEVCVAVWRKNDENITLETVCHDPKLPYH DFILEDAASPKCIMKEKKKPGETFFMCSCSSDECNDNIIFSEE YNTSNPDDNNGAVKFPQLCKFCDVRFSTCDNQKSCMSNCSITS ICEKPQEVCVAVWRKNDENITLETVCHDPKLPYHDFILEDAAS PKCIMKEKKKPGETFFMCSCSSDECNDNIIFSEEYNTIPPHVQ KSVNNDMIVTDNNGAVKFTHTCPPCPAPELLGGPSVFLFPPKP KDTLMISRTPEVTCVVVDVSHEDPEVKFNWYVDGVEVHNAKTK PREEQYNSTYRVVSVLTVLHQDWLNGKEYKCKVSNKALPAPIE KTISKAKGQPREPQVYTLPPSRDELTKNQVSLTCLVKGFYPSD IAVEWESNGQPENNYKTTPPVLDSDGSFFLYSKLTVDKSRWQQ GNVFSCSVMHEALHNHYTQKSLSLSPG | 500 |
| 102 | Protein 134 | IPPHVQKSVNNDMIVTDNNGAVKFPQLCKFCDVRFSTCDNQKS CMSNCSITSICEKPQEVCVAVWRKNDENITLETVCHDPKLPYH DFILEDAASPKCIMKEKKKPGETFFMCSCSSDECNDNIIFSEE YNTSNPDDNNGAVKFPQLCKFCDVRFSTCDNQKSCMSNCSITS ICEKPQEVCVAVWRKNDENITLETVCHDPKLPYHDFILEDAAS | 486 |
| | | PKCIMKEKKKPGETFFMCSCSSDECNDNIIFGGGGSGGGGSGG GGSGTHTCPPCPAPELLGGPSVFLFPPKPKDTLMISRTPEVTC VVVDVSHEDPEVKFNWYVDGVEVHNAKTKPREEQYNSTYRVVS VLTVLHQDWLNGKEYKCKVSNKALPAPIEKTISKAKGQPREPQ VYTLPPSRDELTKNQVSLTCLVKGFYPSDIAVEWESNGQPENN YKTTPPVLDSDGSFFLYSKLTVDKSRWQQGNVFSCSVMHEALH NHYTQKSLSLSPG | |
| 103 | Protein 135 | IPPHVQKSVNNDMIVTDNNGAVKFPQLCKFCDVRFSTCDNQKS CMSNCSITSICEKPQEVCVAVWRKNDENITLETVCHDPKLPYH DFILEDAASPKCIMKEKKKPGETFFMCSCSSDECNDNIIFGGG GSGGGGSGGGGSGGGPQLCKFCDVRFSTCDNQKSCMSNCSITS ICEKPQEVCVAVWRKNDENITLETVCHDPKLPYHDFILEDAAS PKCIMKEKKKPGETFFMCSCSSDECNDNIIFSEEYNTSNPDNG AVKFTHTCPPCPAPELLGGPSVFLFPPKPKDTLMISRTPEVTC VVVDVSHEDPEVKFNWYVDGVEVHNAKTKPREEQYNSTYRVVS VLTVLHQDWLNGKEYKCKVSNKALPAPIEKTISKAKGQPREPQ VYTLPPSRDELTKNQVSLTCLVKGFYPSDIAVEWESNGQPENN YKTTPPVLDSDGSFFLYSKLTVDKSRWQQGNVFSCSVMHEALH NHYTQKSLSLSPG | 486 |
| 104 | Signal peptide | MDWTWRILFLVAAATGTHA | 19 |
| 106 | cDNA encoding Protein 61 | ATTCCTCCACACGTGCAGAAATCCGTGAACAACGACATGATCG TGACCGACAACAATGGCGCCGTGAAGTTCCCTCAGCTGTGCAA GTTCTGCGACGTGCGGTTCTCTACCTGCGACAACCAGAAATCC TGCATGTCCAACTGCTCCATCACCTCCATCTGCGAGAAGCCCC AAGAAGTGTGCGTCGCCGTCTGGCGGAAGAACGACGAGAACAT CACCCTGGAAACCGTGTGCCACGATCCTAAGCTGCCCTACCAC GACTTCATCCTGGAAGATGCCGCCTCTCCTAAGTGCATCATGA AGGAAAAGAAGAAGCCCGGCGAGACTTTCTTCATGTGCAGCTG CTCCTCCGACGAGTGCAACGACAACATCATCTTCTCCGAAGAG TACAACACCAGCAATCCCGACGATAACAACGGGGCTGTGAAAT TCCCACAGCTCTGTAAATTTTGTGATGTGCGGTTCAGCACCTG TGACAATCAAAAGTCTTGCATGAGCAACTGCAGCATCACCAGC ATCTGTGAAAAACCTCAAGAAGTCTGCGTTGCAGTTTGGCGCA AAAATGATGAGAATATCACACTCGAGACAGTGTGTCATGACCC CAAGCTGCCTTATCATGATTTCATCTTGGAGGACGCTGCTAGC CCCAAGTGTATTATGAAGGAAAAAAAGAAACCTGGGGAAACCT TCTTTATGTGCTCCTGCAGCAGCGACGAGTGTAATGATAACAT TATTTTCAGTGAAGAGTATAATACCTCCAATCCTGACAACGGC GCTGTCAAGTTTGGGCCCCCTTGTCCTCCTTGTCCAGCTCCTG AATTTCTCGGCGGACCCTCCGTGTTCCTGTTTCCTCCAAAGCC TAAGGACACCCTGATGATCTCTCGGACCCCTGAAGTGACCTGC | n/a |
| | | GTGGTGGTGGATGTGTCCCAAGAGGACCCTGAGGTGCAGTTCA ATTGGTACGTGGACGGCGTGGAAGTGCACAACGCCAAGACCAA GCCTAGAGAGGAACAGTTCAACTCCACCTACAGAGTGGTGTCC GTGCTGACCGTGCTGCACCAGGATTGGCTGAACGGCAAAGAAT ACAAGTGCAAGGTGTCCAACAAGGGCCTGCCTTCCAGCATCGA AAAGACCATCTCCAAGGCCAAGGGCCAGCCTAGGGAACCCCAG GTGTACACACTGCCTCCAAGCCAAGAGGAAATGACCAAGAACC AGGTGTCCCTGACCTGCCTGGTCAAGGGCTTCTACCCTTCCGA TATCGCCGTGGAATGGGAGAGCAATGGCCAGCCTGAGAACAAC TACAAGACCACACCTCCTGTGCTGGACTCCGACGGCTCCTTCT TTCTGTACTCCCGCCTGACCGTGGACAAGTCCAGATGGCAAGA GGGCAACGTGTTCTCCTGCTCCGTGATGCACGAGGCCCTGCAC AATCACTACACCCAGAAGTCCCTGTCTCTAAGCTTAGGCTGA | |
| 107 | cDNA encoding Protein 96 | ATCCCCCCTCATGTGCAGAAGTCCGTGAACAATGACATGATCG TGACCGATAACAATGGCGCCGTGAAGTTTCCACAGCTGTGCAA GTTCTGTGACGTGAGGTTTAGCACCTGCGATAATCAGAAGTCC TGCATGAGCAACTGTTCTATCACATCCATCTGCGAGAAGCCAC AGGAGGTGTGCGTGGCCGTGTGGCGGAAGAACGACGAGAATAT CACCCTGGAGACAGTGTGCCACGATCCAAAGCTGCCCTACCAT GACTTCATCCTGGAGGATGCTGCCTCTCCTAAGTGTATCATGA AGGAGAAGAAGAAGCCAGGCGAGACCTTCTTTATGTGCTCCTG TTCCAGCGACGAGTGCAACGATAATATCATCTTCAGCGAGGAG TATAACACATCTAATCCCGACGATAACAATGGCGCTGTGAAGT TTCCTCAGCTGTGCAAATTTTGTGACGTGAGATTTTCTACCTG TGATAATCAGAAGAGCTGCATGTCTAACTGTTCCATCACATCT ATTTGTGAAAAACCTCAGGAAGTGTGCGTGGCCGTGTGGAGAA AAAATGATGAAAACATCACCCTGGAAACAGTGTGCCACGATCC TAAGCTGCCATATCACGACTTCATCCTGGAAGACGCTGCCAGC CCAAAATGCATTATGAAAGAGAAGAAGAAGCCCGGTGAGACCT TCTTTATGTGCAGCTGTTCTTCTGATGAATGTAATGACAATAT CATCTTCTCCGAGGAGTATAACACAAGCAATCCTGACAACGGC GCTGTGAAGTTTACCCACACATGCCCACCATGTCCTGCTCCAG AGCTGCTGGGAGGACCATCCGTGTTCCTGTTTCCTCCAAAGCC CAAGGACACCCTGATGATCAGCAGGACCCCCGAGGTGACATGC GTGGTGGTGGACGTGTCTCACGAGGACCCCGAGGTGAAGTTTA ATTGGTACGTGGATGGCGTGGAGGTGCATAACGCTAAGACCAA GCCAAGGGAGGAGCAGTACAACTCTACCTATCGGGTGGTGTCC GTGCTGACAGTGCTGCATCAGGATTGGCTGAATGGCAAGGAGT ATAAGTGCAAGGTGTCCAACAAGGCTCTGCCCGCCCCTATCGA GAAGACCATCAGCAAGGCTAAGGGCCAGCCCAGAGAGCCTCAG GTGTACACACTGCCCCCTTCTCGCGACGAGCTGACCAAGAATC AGGTGTCCCTGACATGTCTGGTGAAGGGCTTCTATCCTTCCGA TATCGCTGTGGAGTGGGAGAGCAACGGACAGCCAGAGAACAAT | n/a |
| | | TACAAGACCACACCACCCGTGCTGGACTCTGATGGCTCCTTCT TTCTGTATAGCAAGCTGACCGTGGACAAGTCTAGGTGGCAGCA GGGCAACGTGTTCTCCTGTTCTGTGATGCACGAGGCCCTGCAC AACCATTACACACAGAAGTCCCTGAGCCTGTCTCCTGGCTGA | |
| 108 | cDNA encoding Protein 101 | ATCCCCCCTCATGTGCAGAAGTCCGTGAACAATGACATGATCG TGACAGATAACAATGGCGCCGTGAAGTTTCCCCAGCTGTGCAA GTTCTGTGACGTGAGGTTTAGCACCTGCGATAACCAGAAGTCC TGCATGAGCAATTGTTCTATCACATCCATCTGCGAGAAGCCTC AGGAGGTGTGCGTGGCCGTGTGGCGGAAGAACGACGAGAATAT CACCCTGGAGACAGTGTGCCACGATCCCAAGCTGCCTTACCAT GACTTCATCCTGGAGGATGCTGCCTCTCCAAAGTGTATCATGA AGGAGAAGAAGAAGCCCGGCGAGACCTTCTTTATGTGCTCCTG TTCCAGCGACGAGTGCAACGATAATATCATCTTCAGCGAGGAG TATAACGATATGATTGTCACAGATAACAATGGCGCTGTGAAGT TTCCACAGCTGTGCAAATTTTGTGACGTGAGATTTTCCACCTG TGATAACCAGAAGAGCTGCATGTCTAATTGTTCCATCACAAGC ATCTGCGAGAAGCCACAGGAAGTGTGCGTGGCCGTGTGGAGAA AAAATGATGAAAACATCACCCTGGAAACAGTGTGCCACGATCC AAAGCTGCCCTATCACGACTTCATCCTGGAAGACGCTGCCAGC CCTAAATGCATTATGAAAGAGAAGAAGAAGCCAGGTGAGACCT TCTTTATGTGCAGCTGTTCTTCTGATGAATGTAATGACAATAT CATCTTCTCCGAGGAGTATAACACCATCCCTCCACACGTGCAG AAGTCTGTGAATAACGATATGATTGTGACCGACAATAACGGTG CTGTGAAGTTTACCCATACATGCCCACCTTGTCCAGCTCCAGA GCTGCTGGGAGGACCATCCGTGTTCCTGTTTCCACCCAAGCCT AAGGACACCCTGATGATCAGCAGGACCCCAGAGGTGACATGCG TGGTGGTGGACGTGTCTCACGAGGACCCCGAGGTGAAGTTTAA CTGGTACGTGGATGGCGTGGAGGTGCATAATGCTAAGACCAAG CCTAGGGAGGAGCAGTACAACTCTACCTATCGGGTGGTGTCCG TGCTGACAGTGCTGCACCAGGACTGGCTGAACGGCAAGGAGTA TAAGTGCAAGGTGTCCAATAAGGCTCTGCCTGCCCCAATTGAG AAGACCATCAGCAAGGCTAAGGGCCAGCCTAGAGAGCCACAGG TGTACACACTGCCTCCATCTCGCGACGAGCTGACCAAGAACCA GGTGTCCCTGACATGTCTGGTGAAGGGCTTCTATCCCTCCGAT ATCGCTGTGGAGTGGGAGAGCAACGGACAGCCTGAGAACAATT ACAAGACCACACCCCCTGTGCTGGACTCTGATGGCTCCTTCTT TCTGTATAGCAAGCTGACCGTGGATAAGTCTAGGTGGCAGCAG GGCAACGTGTTTAGCTGTTCTGTGATGCATGAGGCCCTGCACA ATCATTACACACAGAAGTCCCTGAGCCTGTCTCCAGGCTGA | n/a |
| 109 | cDNA encoding Protein 128 | ATCCCCCCTCATGTGCAGAAGAGCGTCAATAACGACATGATCG TGACCGATAACAATGGCGCCGTGAAGTTCCCACAGCTGTGCAA GTTCTGTGACGTGAGGTTTAGCACCTGCGATAATCAGAAGTCT | n/a |
| | | TGCATGTCCAACTGTAGCATCACATCTATCTGCGAGAAGCCAC AGGAGGTGTGCGTGGCCGTGTGGCGGAAGAACGACGAGAATAT CACCCTGGAGACAGTGTGCCACGATCCAAAGCTGCCCTACCAT GACTTTATCCTGGAGGATGCCGCTAGCCCTAAGTGTATCATGA AGGAGAAGAAGAAGCCAGGCGAGACATTCTTTATGTGCTCTTG TTCCAGCGACGAGTGCAACGATAATATCATCTTCTCCGAGGAG TATAACGATATGATCGTGACAGACAATAACGGCGCCGTGAAGT TTCCTCAGCTGTGCAAATTTTGTGACGTGAGATTTTCTACCTG TGATAATCAGAAGTCCTGCATGAGCAACTGTTCTATCACATCC ATTTGTGAAAAACCTCAGGAAGTGTGCGTGGCCGTGTGGAGAA AGAATGACGAGAACATCACCCTGGAGACAGTGTGCCATGATCC TAAGCTGCCATACCATGACTTCATCCTGGAAGACGCCGCTTCC CCCAAATGCATTATGAAAGAGAAGAAGAAGCCTGGCGAGACAT TCTTCATGTGCAGCTGTTCTTCTGATGAGTGCAACGATAACAT CATCTTTTCTGAGGAGTACAACACCATCCCCCCTCACGTGCAG AAGTCTGTGAACAATGACATGATCGTGACAGATAACAATGGAG CTGTGAAGTTCGGGCCCCCATGCCCTCCATGTCCAGCTCCTGA GTTTCTGGGCGGCCCATCCGTGTTCCTGTTTCCCCCTAAGCCC AAGGACACCCTGATGATCTCCAGGACCCCTGAGGTGACATGCG TGGTGGTGGACGTGAGCCAGGAGGACCCCGAGGTGCAGTTCAA TTGGTACGTGGATGGCGTGGAGGTGCACAACGCCAAGACAAAG CCTAGAGAGGAGCAGTTTAATAGCACCTACCGCGTGGTGTCTG TGCTGACAGTGCTGCATCAGGATTGGCTGAATGGCAAGGAGTA TAAGTGCAAGGTGTCTAACAAGGGCCTGCCAAGCTCTATCGAG AAGACCATCTCCAAGGCTAAGGGACAGCCAAGGGAGCCTCAGG TGTACACACTGCCACCCAGCCAGGAGGAGATGACCAAGAATCA GGTGTCTCTGACATGTCTGGTGAAGGGCTTCTATCCATCTGAC ATCGCTGTGGAGTGGGAGTCCAACGGCCAGCCCGAGAACAATT ACAAGACCACACCTCCAGTGCTGGACAGCGATGGCTCTTTCTT TCTGTATTCCAGGCTGACCGTGGATAAGAGCCGGTGGCAGGAG GGCAACGTGTTTTCCTGTAGCGTGATGCACGAAGCACTGCACA ACCACTACACTCAGAAATCACTGTCACTGTCCCTGGGCTAA | |
| 110 | DNA encoding Kozak sequence (italics) and signal peptide | GCCACCATGGACTGGACCTGGAGAATCCTGTTCCTGGTGGCTG CTGCTACCGGAACACACGCT | n/a |
| 111 | DNA encoding Kozak sequence | GCCACCATGGACTGGACTTGGAGAATCCTGTTCCTGGTCGCCG CCGCAACTGGTACTCATGCT | n/a |
| | (italics) and signal peptide | | |

n/a: not applicable.

### EXAMPLES

The present invention will be more readily understood by referring to the following examples, which are provided to illustrate the invention and are not to be construed as limiting the scope thereof in any manner.

Unless defined otherwise or the context clearly dictates otherwise, all technical and scientific terms used herein have the same meaning as commonly understood by one of ordinary skill in the art to which this invention belongs. It should be understood that any methods and materials similar or equivalent to those described herein can be used in the practice or testing of the invention.

### Example 1 Characterization and Structural Analysis of Known TGFβ Binding Agents

The TGFβRII ectodomain (SEQ ID NO: 1) includes a structured portion, which represents the ligand-binding domain (SEQ ID NO: 2), flanked at both the N-terminal and C-terminal ends by intrinsically disordered regions (IDRs; SEQ ID NOs: 3 and 4 respectively).

Previously, TGFβRII ectodomain fusion molecules were described (WO2017/037634, WO2018/158727). Such fusion molecules include two structured ligand-binding domains (SEQ ID NO: 2) linked together in tandem (head-to-tail) by linkers derived from the IDRs and variants thereof. As reported in WO2018/158727, such polypeptide constructs demonstrated at least 600-fold higher inhibition potency than similar constructs having only a single ligand-binding domain (see WO2018/158727, page 2, lines 27-31, where it is stated: “[a construct] wherein the first region comprises a (TβRII-ECD)-(TβRIIECD) doublet linked at its C-terminus with an antibody constant domain inhibits TGFβ activity with at least 600-fold more potency than a counterpart construct having a single TβRII-ECD linked at its C-terminus with an antibody constant domain (i.e when a second ECD is absent, also referred to herein as a singlet)”). Further, such “doublet” polypeptide constructs (referred to as “T22d35-Fc”) may bind to and neutralize, to varying extents, all three isoforms of TGFβ (that is, TGF-β1, β2, and β3), although TGFβ2 was generally neutralized to a much lesser extent than TGFβ1 and TGFβ3. For example, for the T22d35-Fc-IgG1-v1 (CC) variant, the neutralization potency for TGFβ1 and TGFβ3 was stated to be very similar, whereas this potency was much lower for TGFβ2 (IC50=17.33 nM for TGFβ2 compared to 0.003327 nM and 0.003251 nM, respectively, for TGFβ1 and TGFβ3; see WO2018/158727, page 26, lines 24-27). Similar results were reported for the other variants.

To further study the binding and neutralization properties of such TGFβ binding agents, we chose an exemplary fusion molecule, “T22d35-Fc-IgG1-v1 (CC)”, for further investigation. In this fusion molecule (SEQ ID NO: 6 herein; SEQ ID NO: 14 in WO2018/158727), the linker between the two structured ligand-binding domains is a fusion of the C-terminal and N-terminal IDRs, in that order (the entire linker sequence is shown in SEQ ID NO: 7), and the linker between the second ligand-binding domain and the multimerization domain is the C-terminal IDR (SEQ ID NO: 4) of the TβRII-ECD. In this fusion molecule the multimerization domain is the hIgG1Fc(CC) region (SEQ ID NO: 49).

First, we performed a more detailed characterization of the inhibition potency (IC₅₀) of the T22d35-Fc-IgG1-v1 (CC) fusion for the various isoforms of TGFβ, particularly TGFβ1 and TGFβ3, using a A549 IL-11 release assay. The A549 IL-11 release assay was performed substantially as described (WO2018/158727) and is described in further detail in Example 2 below.

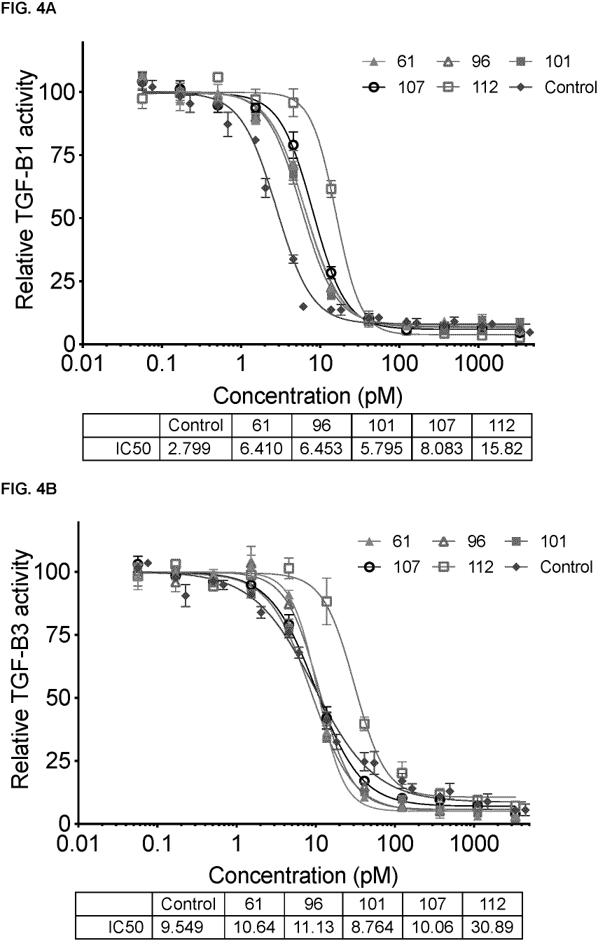

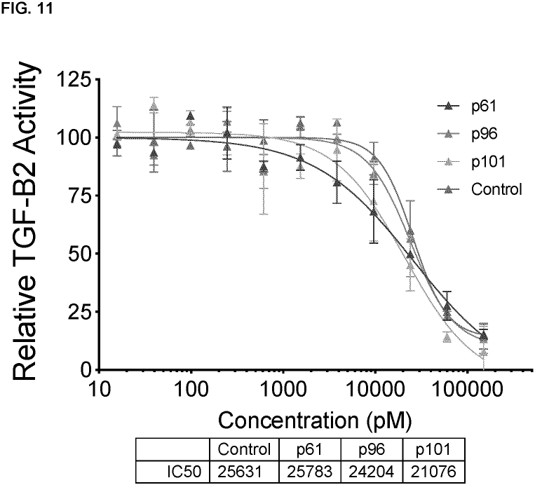

To analyze inhibition potency in greater detail and obtain IC₅₀ values with greater accuracy, we tested more concentrations of TGFβ close to the IC₅₀ (more points on the curve) in the A549 IL-11 release assay. Representative results are shown in FIGS. 4A and 4B and results from the average of 6 experiments are given in Table 3. We determined an average IC₅₀ value of 2.89 pM ± 0.16 for TGFβ1, which is not inconsistent with previous reports (WO2018/158727). However, for TGFβ3, the average IC₅₀ was 8.64 pM ± 0.43, which is significantly higher than expected (i.e., lower inhibition potency than expected). The results indicate approximately 3-3.5 fold higher potency of inhibition for TGFβ1 compared to TGFβ3, indicating preferential inhibition or neutralization of the TGFβ1 ligand by the T22d35-Fc-IgG1-v1 (CC) fusion. It is noted that the inhibition potency for TGFβ2 was significantly lower than for TGFβ1 and TGFβ3, as previously reported (WO2018/158727; FIG. 11).

TABLE 3

IC₅₀ values for T22d35-Fc-IgG1-v1 (CC) (SEQ ID NO: 6).

| TGFβ1 | | TGFβ3 | | IC₅₀ Ratio TGFβ3: TGFβ1 | | n (number of replicates) |
|---|---|---|---|---|---|---|
| Average IC₅₀ (pM) | SEM | Average IC₅₀ (pM) | SEM | Average ratio | SEM | 25 |
| 2.89 | 0.16 | 8.64 | 0.43 | 3.20 | 0.24 | |

SEM: standard error of the mean

These results are consistent with statements in WO2018/158727 that suggest greater inhibition or neutralization of TGFβ1 compared to TGFβ3 (See, for example, page 3, lines 26-28 of WO2018/158727, where it is stated the “Fc-doublet (T22d35-Fc) exhibits a potency enhancement that is at least 970-fold greater for TGFβ1 and at least 240-fold greater for TGFβ3 when compared to a non-Fc fused ECD doublet”).

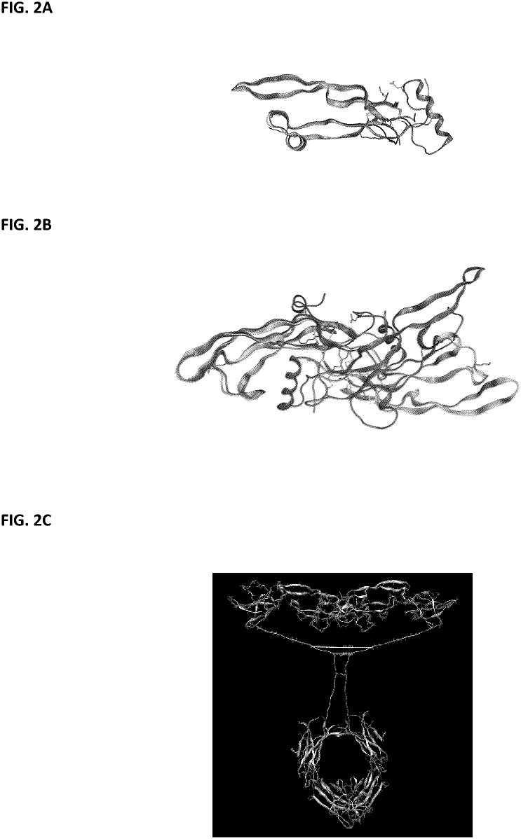

In order to understand better the potential mechanism underlying the preferential inhibition of TGFβ1 ligand versus TGFβ3 ligand by previous fusion constructs, we next conducted a structural comparison of the two ligand isoforms. It should be noted that the ligand-binding domains of the previous fusion constructs interact with essentially identical epitopes on the TGFβ1 and TGFβ3 ligands (Baardsnes, J. et al., 2009). Accordingly, differential affinity for the monomers within the TGFβ dimers does not readily explain the preferential inhibition of TGFβ1 ligand. An overlay of the monomeric structures of TGFβ1 (blue) and TGFβ3 (green) is shown in FIG. 2A. An overlay of TGFβ1 and TGFβ3 dimers as observed in Protein Data Bank (PDB) IDs 3KFD and 1KTZ, respectively, is shown in FIG. 2B (Protein Data Bank (PDB) IDs for the structures are 3KFD and 1KTZ, respectively). It can be seen that, although TGFβ1 and TGFβ3 are very similar at the amino acid sequence level and both monomers adopt a similar extended-cysteine knot fold (FIG. 2A), the arrangement of the two monomers within the biologically active dimers is significantly different when comparing the structures of the two isoforms. It is evident that each ligand isoform has a specific range of dimerization angle. The range of the dimerization angle affects the overall shape, spatial extent and compactness of the dimeric molecule. This difference in shape of the TGFβ1 and TGFβ3 dimers could lead to preferential neutralization of TGFβ1 over TGFβ3 by previous fusion constructs, i.e. the particular spacing of the ligand binding domains within the fusion construct may have led to preferential interactions (preferential avidity) with the TGFβ1 ligand due to that isoform dimer having a distinct shape.

FIG. 2C shows a representative model of a fusion construct (T22d35-Fc-IgG1-v1(CC), SEQ ID NO: 6) bound to TGFβ ligand showing the second ligand-binding domain, second linker, and Fc regions. This model is shown here to illustrate the effects of a short (10 amino acid) second linker. The green line shows that the length of the linker/spacer is short by at least 25 angstroms to allow ligand binding between the attached binding domains. Specifically, the length of the 10 amino acid linker in T22d35-Fc-IgG1-v1(CC) is ~35 Å even in an extended conformation, which is shorter than the optimal linker length by about ~20 Å calculated using molecular modeling. Thus, in this fusion construct, the second ligand binding domains are sterically restricted from accommodating the TGFβ dimer.

It should be noted that the structured ligand-binding domain is the portion of the fusion constructs that contributes to the interaction interface with TGFβ ligands, e.g., TGFβ1 and TGFβ3, and that the linker regions do not directly interact with bound ligand. However, considering the differences in dimeric structure for TGFβ1 and TGFβ3 and the conformational constraints imposed by the linker regions, our analysis suggested that modifying the linker regions may influence the binding properties of the TGFβ binding agent so as to alter its ligand binding specificity. In particular, shortening the first linker region between the ligand-binding domains, and lengthening the second linker region between the second ligand-binding domain and the multimerization domain, might alleviate steric and conformational constraints so as to alter the relative inhibition potencies for the TGFβ1 and TGFβ3 ligands.

### Example 2. Design and Characterization of TGFβ Binding Agents With Altered Isoform Specificity.

We generated molecules with varying linker sequences and lengths to see if modifying the linkers could differentially affect isoform specificity and inhibition potency for the TGFβ ligands, particularly TGFβ1 and TGFβ3. Based on the structural analysis, we focused on molecules with shortened linker portions between the two ligand binding domains (first linker portion) and elongated linker portions between the second ligand binding domain and the multimerization domain (second linker portion). Our goal was to design TGFβ binding agents with less preferential inhibition of TGFβ1 over TGFβ3 (i.e., lower TGFβ3: TGFβ1 IC₅₀ ratio), while maintaining good potency of inhibition overall, in order to provide binding agents with beneficial therapeutic properties for particular disease indications.

A series of TGFβ-binding agents with linkers of varying length and sequence was designed. The structures of representative fusion proteins are summarized in Table 4. Sequences are given in Table 2.

The test binding agents were homodimers, each polypeptide in the homodimer comprising: an N-terminal region comprising the N-terminal IDR of the TGFβRII ectodomain (SEQ ID NO: 3); two TGFβ Receptor Type II (TGFβRII) ligand-binding domains (SEQ ID NO: 2); an 18 amino acid first linker portion between the two ligand-binding domains (SEQ ID NOs: 8 or 12); a 10, 16, or 30 amino acid second linker portion between the second ligand-binding domain and the multimerization domain (SEQ ID NOs: 4, 9, 11, or 15); and the hIgG1Fc(CC) multimerization domain (SEQ ID NO: 49). The T22d35-Fc-IgG1-v1 (CC) fusion (SEQ ID NO: 6; WO2018/158727) was used as a positive control (CTL). The complete sequences of Protein 61 (p61), Protein 96 (p96), Protein 101 (p101), Protein 107 (p107), and Protein 112 (p112) are given in SEQ ID NOs: 81, 84, 87, 89, and 92, respectively (Table 2).

TABLE 4

Structures of representative TGFβ-binding agents in accordance with certain embodiments.

| TGF□ Binding Agent | Linker 1 | | Linker 2 | | Multimerization domain | |
|---|---|---|---|---|---|---|
| | SEQ ID NO | Length (amino acids) | SEQ ID NO | Length (amino acids) | SEQ ID NO | Name |
| T22d35-Fc-IgG1-v1 (CC) (positive control) | 7 | 34 | 4 | 10 | 49 | hIgG1Fc(CC) |
| Protein 61 | 8 | 18 | 9 | 16 | 49 | hIgG1Fc(CC) |
| Protein 93 | 10 | 24 | 9 | 16 | 50 | hIgG4Fc |
| Protein 94 | 10 | 24 | 10 | 24 | 50 | hIgG4Fc |
| Protein 96 | 8 | 18 | 9 | 16 | 49 | hIgG1Fc(CC) |
| Protein 97 | 10 | 24 | 4 | 10 | 49 | hIgG1Fc(CC) |
| Protein 99 | 11 | 30 | 11 | 30 | 50 | hIgG4Fc |
| Protein 101 | 12 | 18 | 11 | 30 | 49 | hIgG1Fc(CC) |
| Protein 106 | 13 | 26 | 13 | 26 | 49 | hIgG1Fc(CC) |
| Protein 107 | 8 | 18 | 4 | 10 | 49 | hIgG1Fc(CC) |
| Protein 108 | 9 | 16 | 9 | 16 | 49 | hIgG1Fc(CC) |
| Protein 109 | 14 | 20 | 9 | 16 | 49 | hIgG1Fc(CC) |
| Protein 112 | 12 | 18 | 15 | 16 | 49 | hIgG1Fc(CC) |
| Protein 113 | 16 | 32 | 16 | 32 | 49 | hIgG1Fc(CC) |
| Protein 115 | 16 | 32 | 16 | 32 | 50 | hIgG4Fc |
| Protein 116 | 16 | 32 | 7 | 34 | 49 | hIgG1Fc(CC) |
| Protein 128 | 12 | 18 | 11 | 30 | 50 | hIgG4Fc |
| Protein 129 | 12 | 18 | 17 | 30 | 49 | hIgG1Fc(CC) |
| Protein 130 | 12 | 18 | 18 | 30 | 49 | hIgG1Fc(CC) |
| Protein 131 | 12 | 18 | 19 | 30 | 49 | hIgG1Fc(CC) |
| Protein 132 | 8 | 18 | 19 | 30 | 49 | hIgG1Fc(CC) |
| Protein 133 | 8 | 18 | 11 | 30 | 49 | hIgG1Fc(CC) |
| Protein 134 | 8 | 18 | 20 | 16 | 49 | hIgG1Fc(CC) |
| Protein 135 | 21 | 18 | 9 | 16 | 49 | hIgG1Fc(CC) |

Production and purification of recombinant fusion molecules. All constructs included the secretion signal sequence MDWTWRILFLVAAATGTHA (SEQ ID NO: 104) at the N-terminus when expressed. The complementary (c) DNAs coding for constructs were prepared synthetically (GeneArt, ThermoFisher Scientific). The cDNAs were cloned into the EcoR1 (5′ end) and BamH1 (3′ end) of the pTT5 mammalian expression plasmid vector (Durocher et al., 2002). Representative cDNA sequences used for expression of fusion proteins are given in Table 2 (SEQ ID NOs: 106-109, used for expression of p61, p96, p101, and p128, respectively). The signal peptide is cleaved off in the cells during expression and is not included in the purified fusion proteins.

Fusion proteins were expressed by transient transfection of Chinese Hamster Ovary (CHO). Briefly, expression plasmids encoding the fusion proteins were each transfected into a 100 mL culture of CHO-3E7 cells in Freestyle F17 medium (Invitrogen) containing 4 mM glutamine and 0.1 % Kolliphor p-1 88 (Sigma).

All cell culture was conducted at 37° C., 5% CO₂. Transfection conditions were as follows: the transfected DNA was comprised of plasmid DNA for expressing the fusion protein and 30% salmon sperm DNA, which was mixed with polyethylenimine-pro (Polyplus) at a ratio = 1:4. At 24 hours post-transfection, 1% Tryptone Nl feed (TekniScience Inc.). 0.5 mM VPA (Sigma) was added and the incubator temperature was dropped to 32° C., 5% CO₂. This was done to promote the production and secretion of the fusion proteins and maintained for 4 days post-transfection (dpt) after which the cultures were harvested. On Day 4, the harvest supernatant was filtered (0.2 µm) and purified using an AKTA pure 25L (GE). Supernatant was loaded onto an MabSelect PrismA protein A column and purified by affinity chromatography. The column was then washed with 8 column volumes of PBS and the protein was eluted with 5 column volumes of 0.1 M sodium citrate, pH 3.2. Fractions were then buffer exchanged into formulation buffer (20 mM L-Histidine, 100 mM NaCl, pH 7) using a HiPrep 26/10 desalting column (GE).

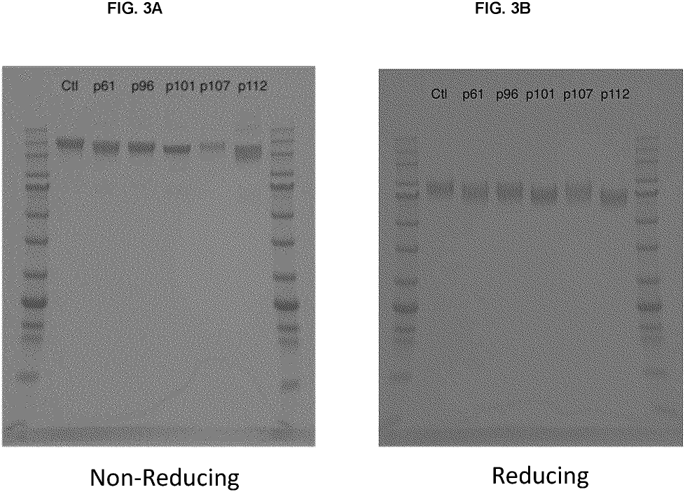

FIGS. 3A and 3B show polyacrylamide gel electrophoresis analysis of samples from purified Proteins 61, 96, 101, 107, and 112 (see the lanes indicated as p61, p96, p101, p107, and p112, respectively) and T22d35-Fc-IgG1-v1 (CC) (Ctl), under both non-reducing (FIG. 3A) and reducing (FIG. 3B) conditions. Proteins (P) were electrophoresed on a 12% Bis-Tris acrylamide gel (NuPAGE™ 12% Bis-Tris Protein Gels, Cat# NP0341BOX, Life Technologies) under both non-reducing and reducing conditions. These fusion proteins are tetravalent, homodimeric TGFβ-binding agents, each comprising two polypeptide chains (i.e., they are homodimers of two polypeptide chains, the first and second polypeptides being the same, and each polypeptide including two ligand-binding domains). The two polypeptide chains are dimerized via disulfide bridges that involve one or more cysteine residues in their multimerization domains, as confirmed by the difference in size under reducing vs. non-reducing conditions.

Inhibition of TGFβ1 and TGFβ3 activities by fusion proteins. To determine the inhibition potencies of Proteins 61, 96, 101, 107, and 112, TGFβ neutralization was assessed, and the inhibition potency was compared to that of a positive control (T22d35-Fc-IgG1-v1 (CC), two TGFβRII-ECD doublets associated via an Fc portion (SEQ ID NO: 6)). It should be noted that a single non-FC-fused TGFβRII ectodomain (SEQ ID NO: 1) does not neutralize any of TGFβ1, β2, or β3 (De Crescenzo et al, 2004). The terms “inhibition potency” and “neutralization potency” are used interchangeably herein.

TGFβ neutralization potencies for the purified fusion proteins were determined using a cell-based signaling assay, specifically an A549 cell/IL-11 release assay using a colorimetric ELISA. Briefly, human A549 lung cancer cells (ATCC-CCL-185, Cedarlane Burlington ON) were seeded in 96-well plates (5 X 10³ cells/well) and incubated at 37° C., 5% CO₂, in a humidified atmosphere. The following day, 10 pM TGFβ in complete media in the absence or presence of increasing concentrations of fusion protein was incubated for 30 min at room temperature (RT) prior to adding to the cells. After 24 hours (h) of incubation, the conditioned medium was harvested and stored at 4° C. The next day, the IL-11 ELISA was performed according to the manufacturer’s instructions (Human IL-11 Duoset ELISA Kit, Cat# DY218, R&D Systems, Inc.). This IL-11 release assay acts as a model of TGFβ-mediated signaling: relative IL-11 release after TGFβ treatment is a measure of TGFβ activity. A decrease in IL-11 release after addition of test fusion protein indicates of TGFβ activity. The data was plotted and analyzed using Prism8 (GraphPad, San Diego) to generate a dose response curve from the absorbance values using 4-parameter fit logistic model (absorbance versus concentration). Values were then normalized to a positive control (TGFβ treatment in the absence of any inhibitor).

Results from a representative set of experiments are shown in FIGS. 4A and 4B, in which the inhibition potency of Proteins 61, 96, 101, 107, and 112 for TGFβ1 and TGFβ3 were compared to the positive control (SEQ ID NO: 6). The highest potency was seen with the positive control. However, the positive control also had the highest IC₅₀ ratio for TGFβ3: TGFβ1 (3.41 in this experiment). In contrast, the TGFβ3: TGFβ1 IC₅₀ ratios for Proteins 61, 96, 101, 107, and 112 were 1.66, 1.72, 1.51, 1.24, and 1.95, respectively, in this experiment (FIGS. 4A-4B).

TABLE 5

Inhibition potencies in A549/IL-11 release assay for representative TGFβ binding agents in accordance with certain embodiments.

| TGFβ Binding Agent | TGFβ1 | | TGFβ3 | | IC₅₀ Ratio TGFβ3: TGFβ1 | | n (number of replicates) |
|---|---|---|---|---|---|---|---|
| | Average IC₅₀ (pM) | SEM | Average IC₅₀ (pM) | SEM | Average ratio | SEM | |
| Protein 61 | 6.38 | 0.29 | 10.63 | 0.05 | 1.67 | 0.07 | 2 |
| Protein 96 | 6.47 | 0.57 | 11.22 | 1.13 | 1.73 | 0.02 | 2 |
| Protein 101 | 6.75 | 0.25 | 9.73 | 0.57 | 1.44 | 0.07 | 5 |
| Protein 106 | 3.27 | 0.15 | 8.27 | 0.63 | 2.54 | 0.31 | 2 |
| Protein 107 | 8.06 | 0.85 | 11.81 | 1.74 | 1.46 | 0.12 | 3 |
| Protein 112 | 15.74 | 0.42 | 29.58 | 1.28 | 1.88 | 0.03 | 2 |
| Protein 113 | 2.19 | 0.37 | 5.73 | 1.17 | 2.49 | 0.24 | 6 |
| Protein 115 | 2.59 | 0.49 | 5.11 | 0.91 | 2.00 | 0.31 | 3 |
| Protein 116 | 2.44 | 0.14 | 6.17 | 0.12 | 2.53 | 0.10 | 3 |
| Protein 128 | 6.67 | 0.44 | 8.42 | 0.72 | 1.28 | 0.12 | 4 |
| Protein 129 | 15.22 | 1.48 | 26.47 | 5.11 | 1.73 | 0.29 | 5 |
| Protein 130 | 7.09 | 0.49 | 8.91 | 1.45 | 1.24 | 0.27 | 3 |
| Protein 131 | 5.71 | 0.79 | 7.70 | 1.25 | 1.31 | 0.33 | 3 |
| Protein 132 | 5.99 | 0.31 | 8.00 | 0.07 | 1.34 | 0.08 | 2 |
| Protein 133 | 4.79 | 0.54 | 8.35 | 0.82 | 1.77 | 0.18 | 3 |
| Protein 134 | 20.24 | 3.44 | 29.57 | 4.17 | 1.54 | 0.47 | 2 |
| Protein 135 | 39.48 | 16.75 | 37.46 | 1.10 | 1.17 | 0.52 | 2 |

SEM: standard error of the mean

The results show that altering the linker regions, specifically shortening the first linker region and lengthening the second linker region, lowered the TGFβ3:TGFβ1 IC₅₀ ratio significantly, indicating reduced preferential inhibition of TGFβ1, while still maintaining inhibition potency in the picomolar range.

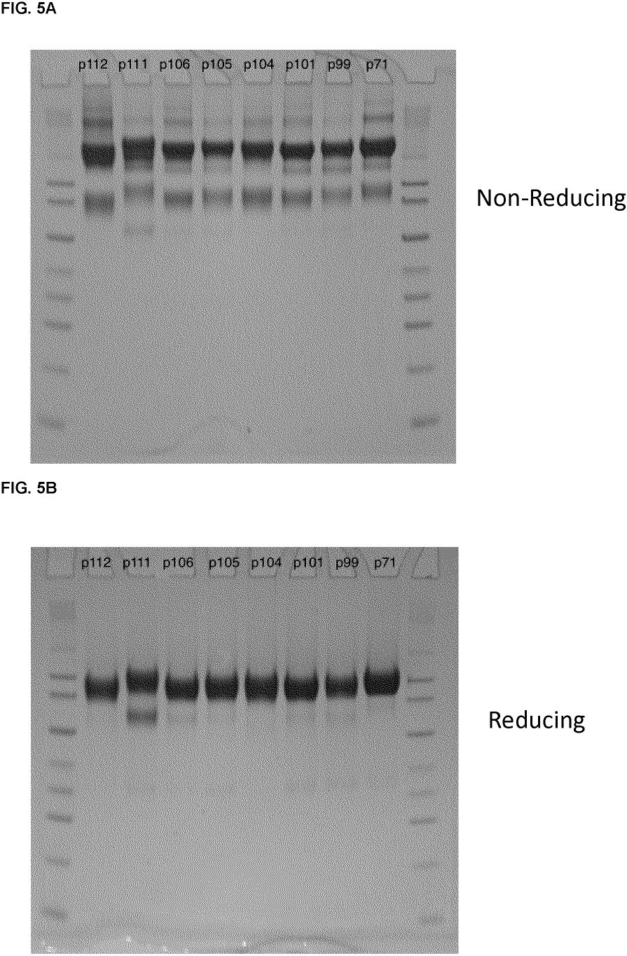

Another set of representative binding agents is shown in FIGS. 5A-5B, which show polyacrylamide gel electrophoresis analysis of samples from purified Proteins 112, 111, 106, 105, 104, 101, 99, and 71, respectively, under non-reducing (FIG. 5A) and reducing (FIG. 5B) conditions.

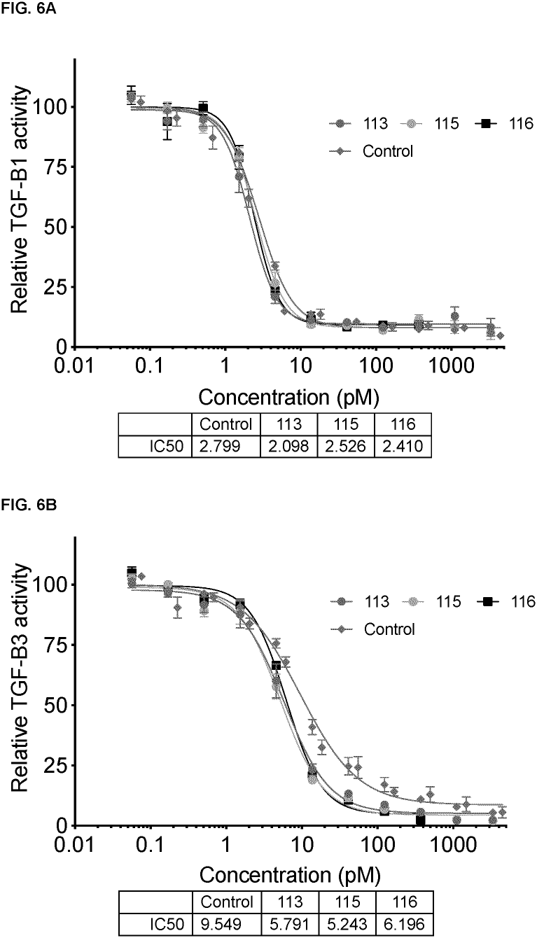

In the representative set of experiments shown in FIGS. 6A-6B, the neutralization potency of Proteins 113, 115 and 116 compared to positive control (SEQ ID NO: 6) is shown. Results are also shown in Table 5. The results show that the inhibition potency for these proteins was comparable to control for TGF β1 but significantly higher for TGFβ3, resulting in a significantly lower TGFβ3:TGFβ1 IC₅₀ ratio.

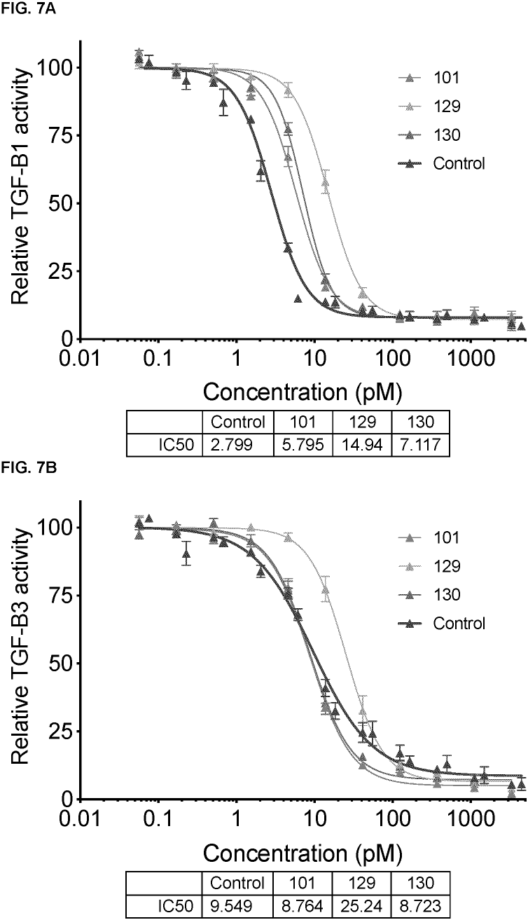

In the representative set of experiments shown in FIGS. 7A-7B, the neutralization potency of Proteins 101, 129, and 130 compared to positive control (SEQ ID NO: 6) is shown. Results are also given in Table 5. The results show that inhibition potency for these proteins was lower for TGF β1 compared to control, and about the same (P101, P130) or lower (P129) for TGFβ3, resulting in a significantly lower TGFβ3:TGFβ1 IC₅₀ ratio.

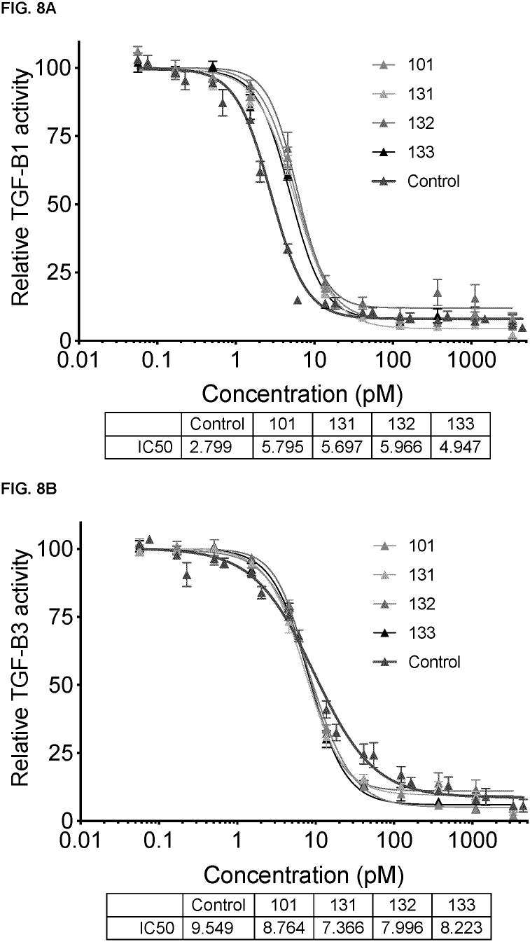

In the representative set of experiments shown in FIGS. 8A-8B, the neutralization potency of Proteins 101, 131, 132, and 133 compared to positive control (SEQ ID NO: 6) is shown. Results are also given in Table 5. The results show that inhibition potency for these proteins was lower for TGFβ1 compared to control, and about the same or higher for TGFβ3, resulting in a significantly lower TGFβ3:TGFβ1 IC₅₀ ratio.

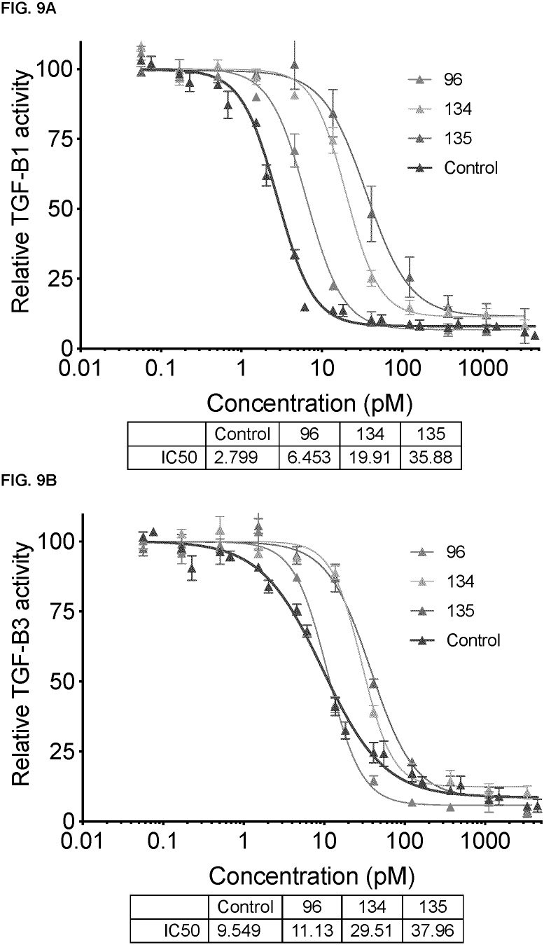

In the representative set of experiments shown in FIGS. 9A-9B, the neutralization potency of Proteins 96, 134, and 135 compared to positive control (SEQ ID NO: 6) is shown. Results are also given in Table 5. The results show that inhibition potency for these proteins was reduced more for TGFβ1 than for TGFβ3 compared to control, so that the TGFβ3:TGFβ1 IC₅₀ ratio was significantly lower than for control. Comparing Proteins 134 and 135 to Protein 96, the results show that replacing either the first linker or the second linker with a Gly-Ser linker significantly reduced inhibition potency for both TGFβ1 and TGFβ3, while maintaining a low TGFβ3:TGFβ1 IC₅₀ ratio.

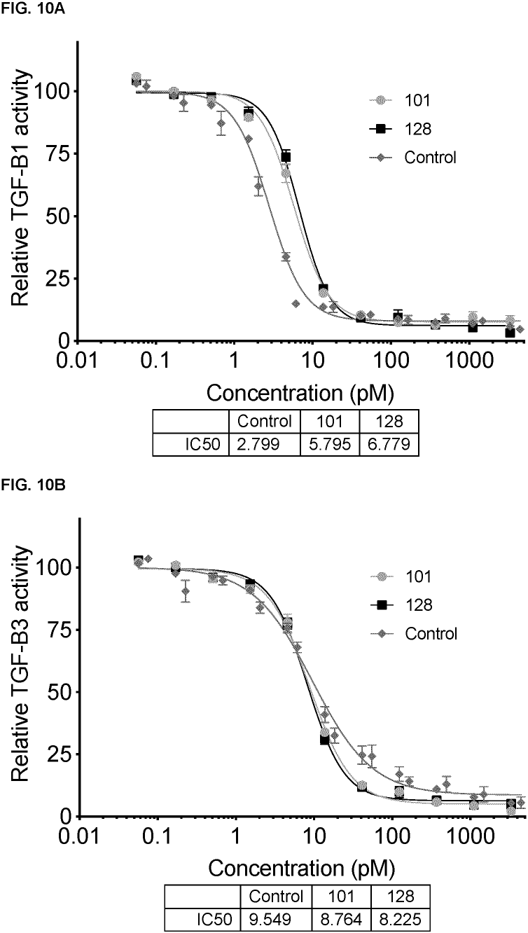

Multimerization domain does not affect TGFβ isoform specificity. TGFβ binding agents having the same TGFβ binding region and differing only in the multimerization domain were tested to see what effect, if any, the multimerization domain has on relative inhibition potency for TGFβ1 and TGFβ3 isoforms. Results are shown in FIGS. 10A-10B, which show neutralization potency of Proteins 101 and 128 compared to positive control (SEQ ID NO: 6), and in Table 5. FIGS. 10A-10B show one representative assay; results averaged from several assays are given in Table 5. The results show that changing the multimerization domain from IgG1 (Protein 101) to IgG4 (Protein 128) had no significant effect on inhibition potency for TGFβ1 and TGFβ3, as expected. The same result was obtained for Proteins 61 and 96, and for Proteins 113 and 115 (Table 5).

Neutralization of the TGFβ2 isoform. We also tested whether the relative inhibition of TGFβ2 was affected, compared to control, by TGFβ binding agents provided herein. A representative set of experiments is shown in FIG. 11. As shown in FIG. 11 for Proteins 61, 96, and 101, the neutralization potency for the TGFβ2 isoform was more than 1000-fold lower than for the TGFβ1 and TGFβ3 isoforms (in other words, IC₅₀ was more than 1000-fold higher), same as for control. The results demonstrate that equalization or shifting of the relative inhibition potencies for TGFβ1 and TGFβ3 isoforms did not have a significant effect on the very low inhibition of the TGFβ2 isoform.

Taken together, the results reported herein show that shortening the first linker region to less than 34 amino acids, and/or lengthening the second linker region to more than 10 amino acids, was effective to lower the TGFβ3:TGFβ1 IC₅₀ ratio for these binding agents, in some cases almost equalizing the inhibition potencies for the two isoforms. It should be noted that in some cases, the TGFβ3:TGFβ1 IC₅₀ ratio was lowered by increasing the inhibition potency for TGFβ3 without adversely affecting the potency for TGFβ1 (e.g., Proteins 113, 115, 116). In other cases, the ratio was lowered primarily by lowering the inhibition potency for TGFβ1 without adversely affecting potency for TGFβ3 (e.g., Proteins 61, 96, 101, 107, 128), although in some cases a slight reduction in TGFβ3 potency was also observed. Nevertheless, all binding agents maintained significantly higher inhibition potency for both TGFβ1 and TGFβ3 than for TGFβ2, consistent with their potential use as therapeutics for the treatment of TGFβ-associated disorders, particularly those mediated by TGFβ3.

### REFERENCES

Akhurst, R.J. and Hata, A. Targeting the TGFβ signaling pathway in disease. Nat. Rev. Drug Discov. 2012; 11: 790-811.

Akhurst, R.J. Targeting TGF-β signaling for therapeutic gain. Cold Spring Harbor Perspect. Biol. 2017; 3; 9: a022301.

Allanore, Y., Simms, R., and Distler. Systemic Sclerosis. Nat. Rev. Dis. Primers. 2015; 1:15002.

Baardsnes, J., Hinck, C.S., Hinck, A. P., O’Connor-McCourt, M.D. TbetaR-II Discriminates the High- And Low-Affinity TGF-beta Isoforms via Two Hydrogen-Bonded Ion Pairs. Biochemistry. 2009; 48(10): 2146-55.

Boocock, G.R., Morrison, J.A., and Popovic, M. Mutations in SBDS are associated with Shwachman-Diamond syndrome. Nature Genetics. 2003; 23:97-101.

Bragado, P., Estrada, Y., and Parikh, F. TGFβ2 dictates disseminated tumour cell fate in target organs through TGFβ-RIII and p38β/β signaling. Nat. Cell. Biol. 2013; 15:1351-1361.

De Crescenzo, G., Pham, P.L., Durocher, Y., Chao, H. and O’Connor-McCourt, M.D. Enhancement of the antagonistic potency of transforming growth factor-beta receptor extracellular domains by coiled coil-induced homo- and heterodimerization. J. Biol. Chem. 2004; 279(25), 26013-26018.

Desmouliere, A., Geinoz, A., Gabbiani, F., and Gabbiani, G. Transforming growth factor-beta 1 induces alpha-smooth muscle actin expression in granulation tissue myofibroblasts and in quiescent and growing cultured fibroblasts. The Journal of Cell Biology. 1993; 122(1):103-11.

Durocher, Y., Perret, S., and Kamen, A. High-level and High-Throughput Recombinant Protein Production by Transient Transfection of Suspension-Growing Human 293-EBNA1 Cells. Nucleic Acids Res. 2002; 30(2): E9.

Gabrielli, A., Avvedminento, E.V., and Krieg, T. Scleroderma. New England Journal of Medicine. 2009; 360: 1989-2003.

Gourh, P., Remmers, E.F., and Satpathy, A. Transforming Growth Factor Beta 3 (TGFB3) - a Novel Systemic Sclerosis Susceptibility Locus Involved in Fibrosis and Th17 Cell Development Identified By Genome-Wide Association Study in African Americans from the Genome Research in African American Scleroderma Patients Consortium. Arthritis Rheumatol. 2017; 69 (suppl 10).

Grutter, C., Wilkinson, T., Turner, R., Podichetty, S., Finch, D., McCourt, M., Loning, S., Jermutus, L., and Grutter, M.G. A Cytokine-Neutralizing Antibody as a Structural Mimetic of 2 Receptor Interactions. Proc. Natl. Acad. Sci. USA. 2008; 105(51): 20251-6.

Herbertz, S., Sawyer, J.S., and Stauber, A.J. Clinical development of galunisertib (LY2157299 monohydrate), a small molecule inhibitor of transforming growth factor-beta signaling pathway. Drug Design, Development and Therapy. 2015; 5: 4479-4499.

Hunzelmann, N. and Krieg, T. Scleroderma: from pathophysiology to novel therapeutic approaches. Experimental Dermatology. 2010; 19(5):393-400.

Komai, I.D. and Okamura, T. Reevaluation of Pluripotent Cytokine TGF-β3 in Immunity. Int. J. Mol. Sci. 2018; 19: 2261.

Kissin, E.Y., Merkel, P.A., and Lafyatis, R. Myofibroblasts and hyalinized collagen as markers of skin disease in systemic sclerosis. Arthritis and Rheumatism. 2006; 54(11):3655-60.

Lacouture, M.E. and Morris, J.C. Cutaneous keratoacanthomas/squamous cell carcinomas associated with neutralization of transforming growth factor β by the monoclonal antibody fresolimumab (GC1008). Cancer Immunol. Immunother. 2015; 64: 437-446.

Langer, J.C., Henckaerts, E., Orenstein, J., and Snoeck, H.W. Quantitative trait analysis reveals transforming growth factor-beta2 as a positive regulator of early hematopoietic progenitor and stem cell function. J. Exp. Med. 2004; 199(1): 5-14.

Lafyatis, R. Transforming growth factor β--at the centre of systemic sclerosis. Nature reviews Rheumatology. 2014; 10(12):706-19.

Leask, A. Targeting the TGFβ, endothelin-1 and CCN2 axis to combat fibrosis in scleroderma. Cell Signal. 2008; 20(8): 1409-14.

Li, M.O., Wan, Y.Y., Sanjabi, S., Robertson, A.K., and Flavell, R.A. Transforming growth factor-β regulation of immune responses. Annu. Rev. Immunol. 2006; 24, 99-146.

Martin, C.J., Datta, A., Littlefield, C., Kalra, A., Chapron, C., Wawersik, S., Dagbay, K.B., Brueckner, C.T., Nikiforov, A., Danehy Jr., F.T., Streich Jr., F.C., Boston, C., Simpson, A., Jackson, J.W., Lin, S., Danek, N., Faucette, R.R., Raman, P., Capili, A.D., Buckler, A., Carven, G.J., and Schurpf, T. Selective Inhibition of TGFβ1 Activation Overcomes Primary Resistance to Checkpoint Blockade Therapy by Altering Tumor Immune Landscape. Sci. Transl. Med. 2020; 12(536): eaay8456.

Mayes, M.D., Lacey, J.V., and Beebe-Dimmer, J. Prevalence, incidence, survival, and disease characteristics of systemic sclerosis in a large US population. Arthritis Rheum. 2003; 48: 2246-2255.

Meng, X.-M., Nikolic-Paterson, D.J., and Lan, H.Y. TGF β: the master regulator of fibrosis. Nat. Rev. Nephrology. 2016; 12: 325-338.

Midgley, A.C., Rogers, M., Hallett, M.B., Clayton, A., Bowen, T., and Phillips, A.O. Transforming growth factor-β1 (TGF-β1)-stimulated fibroblast to myofibroblast differentiation is mediated by hyaluronan (HA)-facilitated epidermal growth factor receptor (EGFR) and CD44 co-localization in lipid rafts. The Journal of Biological Chemistry. 2013; 288(21):14824-38.

Morris, J.C., Tan, A.R., Olencki, T.E., Shapiro, G.I., Dezube, B.J., Reiss, M., Hsu, F.J., Berzofsky, J.A., and Lawrence, D.P. Phase I Study of GC1008 (Fresolimumab): A Human Anti-Transforming Growth Factor-Beta (TGFβ) Monoclonal Antibody in Patients With Advanced Malignant Melanoma or Renal Cell Carcinoma. PLoS One. 2014; 9(3):e90353.

Nanthakumar, D.B., Hatley, R.J.D., and Lemma, S. Dissecting fibrosis: therapeutic insights from the small-molecule toolbox. Nat. Rev. Drug Disc. 2015; 14: 693-720.

Prud’homme, G.J. Pathobiology of transforming growth factor beta in cancer, fibrosis and immunologic disease, and therapeutic considerations. Lab Invest. 2007;87(11):1077-91.

Rice, L.M., Ziemek, J., and Stratton, E.A. A longitudinal biomarker for the extent of skin disease in patients with diffuse cutaneous systemic sclerosis. Arthritis Rheumatol. 2015; 67: 3004-3015.

Rio, P. and Bueren, J.A. TGF-β: a master regulatory of the bone marrow failure puzzle in Fanconi anemia. Stem Cell Investig. 2016; 3: 75 doi: 10.21037/sci.2016.09.17.

Roberts, A.B., Roche, N.S., and Winokur, T.S. Role of transforming growth factor-beta in maintenance of function of cultured neonatal cardiac myocytes. Autocrine action and reversal of damaging effects of interleukin-1. J. Clin. Invest. 1992; 90(5): 2056-2062.

Rogers, Z.R. Shwachman-Diamond Syndrome. 2018. UpToDate; https://www.uptodate.com/contents/shwachman-diamond-syndrome/print.

Tatusova, T.A. and Madden, T. L. Blast 2 sequences - a new tool for comparing protein and nucleotide sequences. FEMS Microbiol Lett. 1999; 174:247-250.

Todd, N.W., Atamas, S.P., Luzina, I.G., and Galvin, J.R. Permanent alveolar collapse is the predominant mechanism in idiopathic pulmonary fibrosis. Expert Review of Respiratory Medicine. 2015; 9(4):411-8.

Vannucchi, A.M., Bianchi, L., and Paoletti, F. A pathobiologic pathway linking thrombopoietin, GATA-1, and TGF-beta1 in the development of myelofibrosis. Blood. 2005; 105: 3493-501.

Varga, J. and Abraham, D. Systemic sclerosis: a prototypic multisystem fibrotic disorder. J. Clin. Invest. 2007; 117: 557-567.

Varga, J. and Pasche, B. Antitransforming growth factor-β therapy in fibrosis: recent progress and implications for systemic sclerosis. Current Opinion in Rheumatology. 2008; 20(6):720-8.

Varga, J. and Whitfield, M.L. Transforming growth factor-beta in systemic sclerosis scleroderma. Frontiers in Bioscience : a journal and virtual library. 2009; S1(1):226-35.

Vincenti, F., Fervenza, F.C., Campbell, K.N., Diaz, M., Gesualdo, L., Nelson, P., Praga, M., Radhakrishnan, J., Sellin, L., Singh, A., Thornley-Brown, D., Veronese, F.V., Accomando, B., Engstrand, S., Ledbetter, S., Lin, J., Neylan, J., Tumlin, J., Focal Segmental Glomerulosclerosis Study Group. A Phase 2, Double-Blind, Placebo-Controlled, Randomized Study of Fresolimumab in Patients With Steroid-Resistant Primary Focal Segmental Glomerulosclerosis. Kidney Int. Rep. 2017; 2(5):800-810.

Wang, J.C., Chang, T.H., and Goldberg, A. Quantitative analysis of growth factor production in the mechanism of fibrosis in agnogenic myeloid metaplasia. Exp. Hematol. 2006; 34(12):1617-23.

Wang, H., Wang, B., Zhang, A., Hassounah, F., Seow, Y., Wood, M., Ma, F., Klein, J.D., Price, S.R., and Wang, X.H. Exosome-Mediated miR-29 Transfer Reduces Muscle Atrophy and Kidney Fibrosis in Mice. Mol. Ther. 2019; 27(3): 571-583.

Zhang, H., Korono, D.E., and O’Connor, K.W. Stem cell defects and bone marrow failure in Fanconi anemia. Cell Stem Cell. 2016; 18: 668-681.

The contents of all documents and references cited herein are hereby incorporated by reference in their entirety.

Although this invention is described in detail with reference to embodiments thereof, these embodiments are offered to illustrate but not to limit the invention. It is possible to make other embodiments that employ the principles of the invention and that fall within its spirit and scope as defined by the claims appended hereto.

What is claimed is:

1. A polypeptide construct useful to inhibit an effect of a Transforming Growth Factor Beta (TGFβ) isoform, the construct comprising:
a TGFβ-binding region, and
a multimerization domain;
wherein the N-terminus of the multimerization domain is joined to the C-terminus of the TGFβ-binding region;
wherein the TGFβ-binding region comprises, in an N- to C- terminal orientation, an N-terminal region, a first TGFβ receptor ligand-binding domain (TGFβR-LBD), a first linker, a second TGFβR ligand-binding domain, and a second linker;
wherein the inhibitory potency of the polypeptide construct for both TGFβ1 isoform activity and TGFβ3 isoform activity is greater than for TGFβ2 isoform activity; and
wherein the first linker and the second linker are selected so that the relative inhibitory potency of the polypeptide construct for TGFβ3 isoform activity compared to TGFβ1 isoform activity (IC₅o ratio for TGFβ3:TGFβ1) is about 2.5:1 or less.

2. The polypeptide construct of claim 1, wherein the relative inhibitory potency of the polypeptide construct for TGFβ3 isoform activity compared to TGFβ1 isoform activity (IC₅₀ ratio for TGFβ3:TGFβ1) is: less than about 2.5:1, about 2.3:1 or less, about 2:1 or less, about 1.8:1 or less, about 1.5:1 or less, about 1.3:1 or less, about 1:1 or less, about 1:1 or less, about 0.8:1 or less, or about 0.5:1 or less.

3. The polypeptide construct of claim 1 or 2, wherein the relative inhibitory potency of the polypeptide construct for TGFβ3 isoform activity compared to TGFβ1 isoform activity (IC₅₀ ratio for TGFβ3:TGFβ1) is from about 1:1 to about 2:1.

4. The polypeptide construct of claim 3, wherein the relative inhibitory potency of the polypeptide construct for TGFβ3 isoform activity compared to TGFβ1 isoform activity (IC₅₀ ratio for TGFβ3:TGFβ1) is about 1.1:1, 1.2:1, 1.3:1, 1.4:1, 1.5:1, 1.6:1, 1.7:1, 1.8:1, or 1.9:1.

5. The polypeptide construct of claim 4, wherein the relative inhibitory potency of the polypeptide construct for TGFβ3 isoform activity compared to TGFβ1 isoform activity (IC₅₀ ratio for TGFβ3:TGFβ1) is from about 1.4:1 to about 1.6:1.

6. The polypeptide construct of claim 5, wherein the relative inhibitory potency of the polypeptide construct for TGFβ3 isoform activity compared to TGFβ1 isoform activity (IC₅₀ ratio for TGFβ3:TGFβ1) is about 1.4:1, about 1.5:1, or about 1.6:1.

7. The polypeptide construct of any one of claims 1 to 6, wherein the polypeptide construct inhibits both TGFβ1 isoform activity and TGFβ3 isoform activity with at least 20-fold, 100-fold, 200-fold, 300-fold, 400-fold, 500-fold, 600-fold, 700-fold, 800-fold, 900-fold, or 1000-fold greater potency than TGFβ2 isoform activity.

8. The polypeptide construct of any one of claims 1 to 7, wherein the first linker is 33 amino acids or shorter.

9. The polypeptide construct of any one of claims 1 to 8, wherein the second linker is 10 amino acids or longer.

10. The polypeptide construct of any one of claims 1 to 9, wherein one or more of the first linker and the second linker comprises or consists of an IDR linker, an IDR linker variant, a hybrid linker, a hybrid linker variant, a truncated linker, a truncated linker variant or an elongated linker.

11. The polypeptide construct of claim 10, wherein one of the first linker and the second linker comprises or consists of a non-IDR linker.

12. The polypeptide construct of any one of claims 1 to 10, wherein both the first linker portion and the second linker portion comprise or consist of an IDR linker, an IDR linker variant, a hybrid linker, a hybrid linker variant, a truncated linker, a truncated linker variant or an elongated linker.

13. The polypeptide construct of any one of claims 1 to 12, wherein the first linker is 10 amino acids or longer, 15 amino acids or longer, or 18 amino acids or longer.

14. The polypeptide construct of any one of claims 1 to 13, wherein the first linker is from about 15 to 33 amino acids long, or from about 18 to about 30 amino acids long.

15. The polypeptide construct of any one of claims 1 to 14, wherein the first linker is about 16, about 18, about 30, or about 32 amino acids long.

16. The polypeptide construct of claim 15, wherein the first linker is 18 amino acids long.

17. The polypeptide construct of claim 15, wherein the first linker is 16 amino acids long.

18. The polypeptide construct of claim 15, wherein the first linker is 30 amino acids long.

19. The polypeptide construct of claim 15, wherein the first linker is 32 amino acids long.

20. The polypeptide construct of any one of claims 1 to 19, wherein the second linker is 35 amino acids or shorter, or from 10 to 34 amino acids long.

21. The polypeptide construct of any one of claims 1 to 20, wherein the second linker is from about 15 to about 35 amino acids long.

22. The polypeptide construct of any one of claims 1 to 21, wherein the second linker is about 16, about 30, about 32, or about 34 amino acids long.

23. The polypeptide construct of claim 22, wherein the second linker is 30 amino acids long.

24. The polypeptide construct of claim 22, wherein the second linker is 16 amino acids long.

25. The polypeptide construct of claim 22, wherein the second linker is 32 amino acids long.

26. The polypeptide construct of claim 22, wherein the second linker is 34 amino acids long.

27. The polypeptide construct of any one of claims 1 to 26, wherein one or more of the first linker and the second linker comprises or consists of the amino acid sequence set forth in any one of SEQ ID NOs: 4 and 8-26, or a sequence at least 80%, at least 85%, at least 90%, at least 95%, at least 96%, at least 97%, at least 98%, or at least 99% identical thereto.

28. The polypeptide construct of claim 27, wherein the first linker comprises or consists of the amino acid sequence set forth in any one of SEQ ID NOs: 8, 9, 10, 11, 12, 13, 14, 16, 21, 22, 23, and 26, or a sequence at least 80%, at least 85%, at least 90%, at least 95%, at least 96%, at least 97%, at least 98%, or at least 99% identical thereto.

29. The polypeptide construct of claim 27 or 28, wherein the second linker comprises or consists of the amino acid sequence set forth in any one of SEQ ID NOs: 4, 9, 11, 15, 17, 18, 19, 20, 22, 23, 24, 25, and 26, or a sequence at least 80%, at least 85%, at least 90%, at least 95%, at least 96%, at least 97%, at least 98%, or at least 99% identical thereto.

30. The polypeptide construct of any one of claims 1 to 29, wherein the first linker comprises or consists of the amino acid sequence set forth in SEQ ID NO: 12.

31. The polypeptide construct of any one of claims 1 to 29, wherein the first linker comprises or consists of the amino acid sequence set forth in SEQ ID NO: 8.

32. The polypeptide construct of any one of claims 1 to 31, wherein the second linker comprises or consists of the amino acid sequence set forth in SEQ ID NO: 11.

33. The polypeptide construct of any one of claims 1 to 31, wherein the second linker comprises or consists of the amino acid sequence set forth in SEQ ID NO: 9.

34. The polypeptide construct of any one of claims 1 to 29, wherein the first linker comprises or consists of an amino acid sequence having:
(a) a deletion of at least one N-terminal amino acid residue in comparison with SEQ ID NO: 3, SEQ ID NO: 12 or SEQ ID NO: 8;
(b) a deletion of at least one C-terminal amino acid residue in comparison with SEQ ID NO: 3, SEQ ID NO: 12 or SEQ ID NO: 8;
(c) a deletion of at least one internal amino acid residue in comparison with SEQ ID NO: 3, SEQ ID NO: 12 or SEQ ID NO: 8; or
(d) one or more substitution in the amino acid sequence in comparison with SEQ ID NO: 3, SEQ ID NO: 12, SEQ ID NO: 8, or of any one of (a) to (c).

35. The polypeptide construct of claim 34, wherein the amino acid deletion is a deletion of 16 amino acids of SEQ ID NO: 3.

36. The polypeptide construct of any one of claims 1 to 35, wherein the second linker comprises or consists of an amino acid sequence having:
(a) a deletion of at least one N-terminal amino acid residue in comparison with SEQ ID NO: 9 or SEQ ID NO: 11;
(b) a deletion of at least one C-terminal amino acid residue in comparison with SEQ ID NO: 9 or SEQ ID NO: 11;
(c) a deletion of at least one internal amino acid residue in comparison with SEQ ID NO: 9 or SEQ ID NO: 11; or
(d) one or more substitution in the amino acid sequence in comparison with SEQ ID NO: 4, 9 or 11, or of any one of (a) to (c).

37. The polypeptide construct of any one of claims 1 to 36, wherein the N-terminal region comprises or consists of an IDR linker, an IDR linker variant, a hybrid linker, a hybrid linker variant, a truncated linker, a truncated linker variant or an elongated linker.

38. The polypeptide construct of any one of claims 1 to 37, wherein the N-terminal region comprises or consists of the amino acid sequence set forth in SEQ ID NO: 3, or a sequence at least 80%, at least 85%, at least 90%, at least 95%, at least 96%, at least 97%, at least 98%, or at least 99% identical thereto.

39. The polypeptide construct of any one of claims 1 to 38, wherein one or more of the first TGFβR-LBD and the second TGFβR-LBD comprises or consists of the amino acid sequence set forth in SEQ ID NO: 2, or a sequence at least 80%, at least 85%, at least 90%, at least 95%, at least 96%, at least 97%, at least 98%, or at least 99% identical thereto.

40. The polypeptide construct of any one of claims 1 to 39, wherein the first TGFβR-LBD and the second TGFβR-LBD are the same or substantially the same.

41. The polypeptide construct of claim 40, wherein both the first TGFβR-LBD and the second TGFβR-LBD comprise or consist of the amino acid sequence set forth in SEQ ID NO: 2, or a sequence at least 80%, at least 85%, at least 90%, at least 95%, at least 96%, at least 97%, at least 98%, or at least 99% identical thereto.

42. The polypeptide construct of any one of claims 1 to 41, wherein the multimerization domain allows dimerization of the polypeptide construct with a second polypeptide construct as defined in any one of claims 1 to 41, in a non-covalent manner.

43. The polypeptide construct of any one of claims 1 to 41, wherein the multimerization domain allows dimerization of the polypeptide construct with a second polypeptide construct as defined in any one of claims 1 to 41, in a covalent manner.

44. The polypeptide construct of any one of claims 1 to 43, wherein the multimerization domain comprises one or more constant region of an antibody.

45. The polypeptide construct of claim 44, wherein the multimerization domain comprises the second constant domain (C_{H}2) and/or the third constant domain (C_{H}3) of an antibody heavy chain.

46. The polypeptide construct of any one of claims 1 to 45, wherein the multimerization domain comprises an Fc region of an antibody heavy chain.

47. The polypeptide construct of any one of claims 44 to 46, wherein the antibody is an IgG antibody.

48. The polypeptide construct of claim 47, wherein the IgG antibody is an IgG1, IgG2, IgG3 or IgG4 antibody, optionally a human antibody.

49. The polypeptide construct of claim 48, wherein the multimerization domain has at least 80%, at least 85%, at least 90%, at least 95%, at least 96%, at least 97%, at least 98%, or at least 99% sequence identity with a human IgG1, IgG2, IgG3 or IgG4 constant region.

50. The polypeptide construct of any one of claims 1 to 49, wherein the multimerization domain comprises cysteine residues for crosslinking of the polypeptide construct with a second polypeptide construct as defined in any one of claims 1 to 49.

51. The polypeptide construct of claim 50, wherein the multimerization domain comprises at least two cysteine residues for forming a disulfide bridge with the second polypeptide construct.

52. The polypeptide construct of any one of claims 1 to 51, wherein the multimerization domain is engineered to reduce aggregation or to modulate stability of a dimer or multimer of the polypeptide construct.

53. The polypeptide construct of any one of claims 1 to 52, wherein the multimerization domain comprises or consists of the amino acid sequence set forth in SEQ ID NO:49.

54. The polypeptide construct of any one of claims 1 to 52, wherein the multimerization domain comprises or consists of the amino acid sequence set forth in SEQ ID NO:50.

55. The polypeptide construct of any one of claims 1 to 52, wherein the multimerization domain comprises or consists of the amino acid sequence set forth in any one of SEQ ID NOs: 49-80 or a sequence at least 80%, at least 85%, at least 90%, at least 95%, at least 96%, at least 97%, at least 98%, or at least 99% identical thereto.

56. The polypeptide construct of any one of claims 1 to 55, wherein the TGFβ-binding region comprises or consists of the amino acid sequence set forth in any one of SEQ ID NOs: 27-48, or a sequence at least 80%, at least 85%, at least 90%, at least 95%, at least 96%, at least 97%, at least 98%, or at least 99% identical thereto.

57. The polypeptide construct of any one of claims 1 to 56, wherein the TGFβ-binding region comprises or consists of the amino acid sequence set forth in SEQ ID NO: 27.

58. The polypeptide construct of any one of claims 1 to 56, wherein the TGFβ-binding region comprises or consists of the amino acid sequence set forth in SEQ ID NO: 29.

59. The polypeptide construct of any one of claims 1 to 56, wherein the TGFβ-binding region comprises or consists of the amino acid sequence set forth in SEQ ID NO: 32.

60. The polypeptide construct of any one of claims 1 to 56, wherein the TGFβ-binding region comprises or consists of the amino acid sequence set forth in SEQ ID NO: 40.

61. The polypeptide construct of any one of claims 1 to 56, wherein the TGFβ-binding region comprises or consists of the amino acid sequence set forth in SEQ ID NO: 41.

62. The polypeptide construct of any one of claims 1 to 61, wherein the polypeptide construct comprises or consists of the amino acid sequence set forth in any one of SEQ ID NOs: 81 to 103 and 105, or a sequence at least 80%, at least 85%, at least 90%, at least 95%, at least 96%, at least 97%, at least 98%, or at least 99% identical thereto.

63. The polypeptide construct of any one of claims 1 to 61, wherein the polypeptide construct comprises or consists of the amino acid sequence set forth in SEQ ID NO: 81, or a sequence at least 80%, at least 85%, at least 90%, at least 95%, at least 96%, at least 97%, at least 98%, or at least 99% identical thereto.

64. The polypeptide construct of any one of claims 1 to 61, wherein the polypeptide construct comprises or consists of the amino acid sequence set forth in SEQ ID NO: 84, or a sequence at least 80%, at least 85%, at least 90%, at least 95%, at least 96%, at least 97%, at least 98%, or at least 99% identical thereto.

65. The polypeptide construct of any one of claims 1 to 61, wherein the polypeptide construct comprises or consists of the amino acid sequence set forth in SEQ ID NO: 87, or a sequence at least 80%, at least 85%, at least 90%, at least 95%, at least 96%, at least 97%, at least 98%, or at least 99% identical thereto.

66. The polypeptide construct of any one of claims 1 to 61, wherein the polypeptide construct comprises or consists of the amino acid sequence set forth in SEQ ID NO: 95, or a sequence at least 80%, at least 85%, at least 90%, at least 95%, at least 96%, at least 97%, at least 98%, or at least 99% identical thereto.

67. The polypeptide construct of any one of claims 1 to 61, wherein the polypeptide construct comprises or consists of the amino acid sequence set forth in SEQ ID NO: 96, or a sequence at least 80%, at least 85%, at least 90%, at least 95%, at least 96%, at least 97%, at least 98%, or at least 99% identical thereto.

68. The polypeptide construct of any one of claims 1 to 67, wherein the polypeptide construct further comprises an amino acid sequence suitable for expression, detection and/or purification of the TGFβ binding agent.

69. The polypeptide construct of claim 68, wherein the polypeptide construct further comprises a signal peptide having the sequence set forth in SEQ ID NO: 104, or a sequence substantially identical thereto.

70. The polypeptide construct of any one of claims 1 to 69, wherein the polypeptide construct is a dimeric polypeptide comprising a first and a second polypeptide construct as defined in any one of claims 1 to 69 linked between respective multimerization domains by at least one disulfide bridge.

71. The polypeptide construct of claim 70, wherein the first and the second polypeptide construct comprise the same or substantially the same amino acid sequence.

72. The polypeptide construct of claim 70, wherein the first and the second polypeptide construct comprise different amino acid sequences.

73. The polypeptide construct of claim 72, wherein the first and the second polypeptide construct comprise the same or substantially the same multimerization domains, and different TGFβ-binding regions.

74. The polypeptide construct of any one of claims 70 to 73, wherein the first polypeptide and/or the second polypeptide construct further comprises a site for conjugation.

75. The polypeptide construct of claim 74, wherein the first polypeptide and/or the second polypeptide construct is conjugated with a targeting agent, a therapeutic moiety, a detectable moiety, or a diagnostic moiety.

76. The polypeptide construct of claim 75, wherein the targeting agent, the therapeutic moiety, the detectable moiety, or the diagnostic moiety comprises an antibody or antigen binding fragment thereof, a binding agent having affinity for another member of the TGFβ family or for another therapeutic target, a radiotherapy agent, an imaging agent, a fluorescent moiety, a cytotoxic agent, an anti-mitotic drug, a nanoparticle-based carrier, a polymer-conjugated drug, a nanocarrier, an imaging agent, a stabilizing agent, a drug, a nanocarrier, or a dendrimer.

77. A polypeptide construct comprising from N-terminus to C-terminus: (i) an amino acid sequence consisting of the amino acid sequence of SEQ ID NO:40; and (ii) an Fc region of human IgG1.

78. A nucleic acid molecule encoding the polypeptide construct of any one of claims 1 to 77.

79. The nucleic acid molecule of claim 78, wherein the nucleic acid molecule encodes the polypeptide construct in a form that is secretable by a selected expression host.

80. A nucleic acid molecule encoding at least one polypeptide having the amino acid sequence set forth in any one of SEQ ID NOs: 81 to 103 and 105, or a sequence substantially identical thereto.

81. A nucleic acid molecule having the sequence set forth in any one of SEQ ID NOs: 106-109, or a sequence substantially identical thereto.

82. The nucleic acid molecule of claim 80, further comprising, at the 5′ end, the sequence set forth in SEQ ID NO: 110 or SEQ ID NO: 111, or a sequence substantially identical thereto.

83. A vector comprising the nucleic acid molecule of any one of claims 78 to 82.

84. A cellular host comprising the nucleic acid molecule of any one of claims 78 to 82 or the vector of claim 83.

85. A TGFβ binding agent comprising:
a first polypeptide construct as defined in any one of claims 1 to 77, and
a second polypeptide construct as defined in any one of claims 1 to 77;
wherein the first polypeptide construct and the second polypeptide construct are associated together through their respective multimerization domains
wherein the inhibitory potency of the TGFβ binding agent for both TGFβ1 isoform activity and TGFβ3 isoform activity is greater than for TGFβ2 isoform activity; and
wherein the first linker and the second linker are selected so that the relative inhibitory potency of the TGFβ binding agent for TGFβ3 isoform activity compared to TGFβ1 isoform activity (IC₅o ratio for TGFβ3 TGFβ1) is about 2.5:1 or less.

86. The TGFβ binding agent of claim 85, wherein the relative inhibitory potency of the TGFβ binding agent for TGFβ3 isoform activity compared to TGFβ1 isoform activity (IC₅o ratio for TGFβ3:TGFβ1) is: less than about 2.5:1, about 2.3:1 or less, about 2:1 or less, about 1.8:1 or less, about 1.5:1 or less, about 1.3:1 or less, about 1:1 or less, about 1:1 or less, about 0.8:1 or less, or about 0.5:1 or less.

87. The TGFβ binding agent of claim 85 or 86, wherein the relative inhibitory potency of the TGFβ binding agent for TGFβ3 isoform activity compared to TGFβ1 isoform activity (IC₅o ratio for TGFβ3:TGFβ1) is from about 1:1 to about 2:1.

88. The TGFβ binding agent of claim 87, wherein the relative inhibitory potency of the TGFβ binding agent for TGFβ3 isoform activity compared to TGFβ1 isoform activity (IC₅o ratio for TGFβ3:TGFβ1) is about 1.1:1, 1.2:1, 1.3:1, 1.4:1, 1.5:1, 1.6:1, 1.7:1, 1.8:1, or 1.9:1.

89. The TGFβ binding agent of claim 88, wherein the relative inhibitory potency of the TGFβ binding agent for TGFβ3 isoform activity compared to TGFβ1 isoform activity (IC₅o ratio for TGFβ3:TGFβ1) is from about 1.4:1 to about 1.6:1.

90. The TGFβ binding agent of claim 89, wherein the relative inhibitory potency of the TGFβ binding agent for TGFβ3 isoform activity compared to TGFβ1 isoform activity (IC₅o ratio for TGFβ3:TGFβ1) is about 1.4:1, about 1.5:1, or about 1.6:1.

91. The TGFβ binding agent of any one of claims 85 to 90, wherein the TGFβ binding agent inhibits both TGFβ1 isoform activity and TGFβ3 isoform activity with at least 20-fold, 100-fold, 200-fold, 300-fold, 400-fold, 500-fold, 600-fold, 700-fold, 800-fold, or 900-fold greater potency than TGFβ2 isoform activity.

92. The TGFβ binding agent of any one of claims 85 to 91, wherein the TGFβ binding agent is a dimer wherein the first polypeptide construct and the second polypeptide construct are linked between their respective multimerization domains by at least one disulfide bridge.

93. The TGFβ binding agent of claim 92, wherein the TGFβ binding agent is a homodimer, the first polypeptide construct and the second polypeptide construct being the same or substantially the same.

94. The TGFβ binding agent of claim 93, wherein the first polypeptide construct and the second polypeptide construct comprise or consist of the sequence set forth in any one of SEQ ID NOs: 81, 84, 87, or 96, or a sequence at least 80%, at least 85%, at least 90%, at least 95%, at least 96%, at least 97%, at least 98%, or at least 99% identical thereto.

95. The TGFβ binding agent of claim 93 or 94, wherein the first polypeptide construct and the second polypeptide construct comprise or consist of the sequence set forth in SEQ ID NO:87.

96. The TGFβ binding agent of claim 93 or 94, wherein the first polypeptide construct and the second polypeptide construct comprise or consist of the sequence set forth in SEQ ID NO:96.

97. The TGFβ binding agent of claim 93, wherein the first polypeptide construct and the second polypeptide construct comprises or consists of the sequence set forth in SEQ ID NO:95, or a sequence at least 80%, at least 85%, at least 90%, at least 95%, at least 96%, at least 97%, at least 98%, or at least 99% identical thereto.

98. The TGFβ binding agent of claim 97, wherein the first polypeptide construct and the second polypeptide construct comprise or consist of the sequence set forth in SEQ ID NO:95.

99. The TGFβ binding agent of claim 92, wherein the TGFβ binding agent is a heterodimer, the first polypeptide construct and the second polypeptide construct comprising different amino acid sequences.

100. The TGFβ binding agent of claim 99, wherein the first polypeptide construct and the second polypeptide construct comprising the same or substantially the same multimerization domains, and different TGFβ-binding regions.

101. The TGFβ binding agent of claim 100, wherein the different TGFβ-binding regions comprise or consist of the amino acid sequence set forth in any one of SEQ ID NOs: 27, 29, 87 and 96, or a sequence at least 80%, at least 85%, at least 90%, at least 95%, at least 96%, at least 97%, at least 98%, or at least 99% identical thereto.

102. The TGFβ binding agent of claim 100, wherein the different TGFβ-binding regions comprise or consist of the amino acid sequence set forth in SEQ ID NO:95, or a sequence at least 80%, at least 85%, at least 90%, at least 95%, at least 96%, at least 97%, at least 98%, or at least 99% identical thereto.

103. A TGFβ binding agent comprising:
a first polypeptide construct comprising from N-terminus to C-terminus: (i) an amino acid sequence consisting of the amino acid sequence of SEQ ID NO:40; and (ii) a first Fc region of human IgG1, and
a second polypeptide construct comprising from N-terminus to C-terminus: (i) an amino acid sequence consisting of the amino acid sequence of SEQ ID NO:40; and (ii) a second Fc region of human IgG1;
wherein the first polypeptide construct and the second polypeptide construct are linked together through the first and second Fc region of human IgG1.

104. The TGFβ binding agent of claim 103, wherein the inhibitory potency of the TGFβ binding agent for both TGFβ1 isoform activity and TGFβ3 isoform activity is greater than for TGFβ2 isoform activity; and the relative inhibitory potency of the TGFβ binding agent for TGFβ3 isoform activity compared to TGFβ1 isoform activity (IC₅o ratio for TGFβ3 TGFβ1) is about 2.5:1 or less.

105. A TGFβ binding agent, wherein the TGFβ binding agent is a homodimer of the polypeptide construct of any one of claims 1 to 77.

106. A pharmaceutical composition comprising the polypeptide construct of any one of claims 1 to 77 or the TGFβ binding agent of any one of claims 85 to 105 and a pharmaceutically acceptable carrier, diluent or excipient.

107. The pharmaceutical composition of claim 106, wherein the composition comprises the polypeptide construct of claim 64 or 67 or the TGFβ binding agent of claim 95 or 96, or a combination thereof.

108. The pharmaceutical composition of claim 106, wherein the composition comprises the polypeptide construct of claim 66 or the TGFβ binding agent of claim 98, or a combination thereof.

109. The pharmaceutical composition of claim 106, wherein the composition comprises the polypeptide construct of claim 66 or the TGFβ binding agent of claim 103, or a combination thereof.

110. The pharmaceutical composition of any one of claims 106 to 109, wherein the composition is formulated for administration by injection or infusion.

111. The pharmaceutical composition of claim 110, wherein the composition is formulated for intravenous, subcutaneous, intraperitoneal, or intramuscular administration.

112. A method of manufacturing the polypeptide construct of any one of claims 1 to 77 or the TGFβ binding agent of any one of claims 85 to 105, the method comprising expressing the first polypeptide construct and/or the second polypeptide construct in a cell.

113. The method of claim 112, further comprising culturing the cell and isolating and/or purifying the polypeptide construct or the TGFβ binding agent expressed in the cell.

114. The method of claim 113, wherein the polypeptide construct and/or the TGFβ binding agent is secreted by the cell, and the polypeptide construct and/or the TGFβ binding agent is obtained from medium in which the cell is cultured.

115. A method of treating or preventing a TGFβ-associated disease or condition in a subject in need thereof, the method comprising administering the polypeptide construct of any one of claims 1 to 77 or the TGFβ binding agent of any one of claims 85 to 105 to the subject, such that the TGFβ-associated disease or condition is treated or prevented in the subject.

116. The method of claim 115, wherein the subject is a mammal.

117. The method of claim 116, wherein the mammal is a human.

118. The method of any one of claims 115 to 117, wherein the subject has, or is suspected of having, a disease or condition mediated by TGFβ1 and/or TGFβ3.

119. The method of any one of claims 115 to 118, wherein the subject has, or is suspected of having, a disease or condition mediated by TGFβ3.

120. A method of treating or preventing a disease or condition mediated by TGFβ1 and/or TGFβ3 in a subject, the method comprising administering the polypeptide construct of any one of claims 1 to 77 or the TGFβ binding agent of any one of claims 85 to 105 to the subject, such that the disease or condition mediated by TGFβ1 and/or TGFβ3 is treated or prevented in the subject.

121. The method of claim 120, wherein the disease is mediated by TGFβ3.

122. The method of any one of claims 115 to 121, wherein the disease or condition is characterized by overexpression or overactivation of TGFβ1 and/or TGFβ3.

123. The method of any one of claims 115 to 122, wherein the disease or condition is fibrosis.

124. The method of claim 123, wherein the fibrosis is pulmonary fibrosis, idiopathic pulmonary fibrosis, renal fibrosis, liver fibrosis, lung fibrosis, kidney fibrosis, bone marrow fibrosis, systemic sclerosis, skin fibrosis, heart fibrosis, myelofibrosis, a fibroproliferative disorder or a connective tissue disorder.

125. The method of any one of claims 115 to 122, wherein the disease or condition is a bone marrow failure disease.

126. The method of claim 125, wherein the disease or condition is Shwachman-Bodian-Diamond syndrome or Fanconi anemia.

127. A method of manufacturing of the polypeptide construct of any one of claims 1 to 77 or the TGFβ binding agent of any one of claims 85 to 105, comprising culturing the host cell of claim 84 under conditions suitable for protein expression; and harvesting the polypeptide construct of any one of claims 1 to 77 or the TGFβ binding agent of any one of claims 85 to 105.

128. A polypeptide construct or a TGFβ binding agent produced by the method of claim 127.


SEQUENCE LISTING (111 sequences)

SEQ ID NO: 1 - protein, 136 aa, Artificial Sequence
  hTGF-beta RII-ECD
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 2 - protein, 102 aa, Artificial Sequence
  TGF-beta R-LBD
PQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIF

SEQ ID NO: 3 - protein, 24 aa, Artificial Sequence
  N-terminal IDR
IPPHVQKSVNNDMIVTDNNGAVKF

SEQ ID NO: 4 - protein, 10 aa, Artificial Sequence
  C-terminal IDR
SEEYNTSNPD

SEQ ID NO: 5 - protein, 272 aa, Artificial Sequence
  TGF-beta -binding region from T22d35-Fc-IgG1-v1(CC)
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 6 - protein, 496 aa, Artificial Sequence
  T22d35-Fc-IgG1-v1(CC)(Control)
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 7 - protein, 34 aa, Artificial Sequence
  Linker
SEEYNTSNPDIPPHVQKSVNNDMIVTDNNGAVKF

SEQ ID NO: 8 - protein, 18 aa, Artificial Sequence
  Linker
SEEYNTSNPDDNNGAVKF

SEQ ID NO: 9 - protein, 16 aa, Artificial Sequence
  Linker
SEEYNTSNPDNGAVKF

SEQ ID NO: 10 - protein, 24 aa, Artificial Sequence
  Linker
SEEYNTSNPDNDMIVTDNNGAVKF

SEQ ID NO: 11 - protein, 30 aa, Artificial Sequence
  Linker
SEEYNTIPPHVQKSVNNDMIVTDNNGAVKF

SEQ ID NO: 12 - protein, 18 aa, Artificial Sequence
  Linker
SEEYNDMIVTDNNGAVKF

SEQ ID NO: 13 - protein, 26 aa, Artificial Sequence
  Linker
SEEYPHVQKSVNNDMIVTDNNGAVKF

SEQ ID NO: 14 - protein, 20 aa, Artificial Sequence
  Linker
SEEYNTSNPDVTDNNGAVKF

SEQ ID NO: 15 - protein, 16 aa, Artificial Sequence
  Linker
SEEYMIVTDNNGAVKF

SEQ ID NO: 16 - protein, 32 aa, Artificial Sequence
  Linker
SEEYNTSNPDPHVQKSVNNDMIVTDNNGAVKF

SEQ ID NO: 17 - protein, 30 aa, Artificial Sequence
  Linker
GGGGSGGGGSGGGGSGGGGSGGGGSGGGGS

SEQ ID NO: 18 - protein, 30 aa, Artificial Sequence
  Linker
SEEYNTGGGGSGGGGSGGGGSGGGGSGGGG

SEQ ID NO: 19 - protein, 30 aa, Artificial Sequence
  Linker
SEEYNTSNPDVQKSVNNDMIVTDNNGAVKF

SEQ ID NO: 20 - protein, 16 aa, Artificial Sequence
  Linker
GGGGSGGGGSGGGGSG

SEQ ID NO: 21 - protein, 18 aa, Artificial Sequence
  Linker
GGGGSGGGGSGGGGSGGG

SEQ ID NO: 22 - protein, 17 aa, Artificial Sequence
  Linker
SEEYDMIVTDNNGAVKF

SEQ ID NO: 23 - protein, 19 aa, Artificial Sequence
  Linker
SEEYNNDMIVTDNNGAVKF

SEQ ID NO: 24 - protein, 34 aa, Artificial Sequence
  Linker
GGGGSGGGGSGGGGSGGGGSGGGGSGGGGSGGGG

SEQ ID NO: 25 - protein, 34 aa, Artificial Sequence
  Linker
SEEYNTSNPDGGGGSGGGGSGGGGSGGGGSGGGG

SEQ ID NO: 26 - protein, 26 aa, Artificial Sequence
  Linker
SEEYNTSNPDVNNDMIVTDNNGAVKF

SEQ ID NO: 27 - protein, 262 aa, Artificial Sequence
  TGF-beta -binding region - Protein 61
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDNGAVKF

SEQ ID NO: 28 - protein, 268 aa, Artificial Sequence
  TGF-beta -binding region - Protein 93
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDNGAVKF

SEQ ID NO: 29 - protein, 262 aa, Artificial Sequence; identical to SEQ ID NO: 27
  TGF-beta -binding region - Protein 96

SEQ ID NO: 30 - protein, 262 aa, Artificial Sequence
  TGF-beta -binding region - Protein 97
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 31 - protein, 288 aa, Artificial Sequence
  TGF-beta -binding region - Protein 99
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTIPPHVQKSVNNDMIVTDNNGAVKF

SEQ ID NO: 32 - protein, 276 aa, Artificial Sequence
  TGF-beta -binding region - Protein 101
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTIPPHVQKSVNNDMIVTDNNGAVKF

SEQ ID NO: 33 - protein, 280 aa, Artificial Sequence
  TGF-beta -binding region - Protein 106
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYPHVQKSVNNDMIVTDNNGAVKF

SEQ ID NO: 34 - protein, 256 aa, Artificial Sequence
  TGF-beta -binding region - Protein 107
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 35 - protein, 260 aa, Artificial Sequence
  TGF-beta -binding region - Protein 108
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDNGAVKF

SEQ ID NO: 36 - protein, 264 aa, Artificial Sequence
  TGF-beta -binding region - Protein 109
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDNGAVKF

SEQ ID NO: 37 - protein, 262 aa, Artificial Sequence
  TGF-beta -binding region - Protein 112
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYMIVTDNNGAVKF

SEQ ID NO: 38 - protein, 292 aa, Artificial Sequence
  TGF-beta -binding region - Protein 113
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDPHVQKSVNNDMIVTDNNGAVKF

SEQ ID NO: 39 - protein, 292 aa, Artificial Sequence; identical to SEQ ID NO: 38
  TGF-beta -binding region - Protein 115

SEQ ID NO: 40 - protein, 294 aa, Artificial Sequence
  TGF-beta -binding region - Protein 116
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDIPPHVQKSVNNDMIVTDNNGAVKF

SEQ ID NO: 41 - protein, 276 aa, Artificial Sequence; identical to SEQ ID NO: 32
  TGF-beta -binding region - Protein 128

SEQ ID NO: 42 - protein, 276 aa, Artificial Sequence
  TGF-beta -binding region - Protein 112
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFGGGGSGGGGSGGGGSGGGGSGGGGSGGGGS

SEQ ID NO: 43 - protein, 276 aa, Artificial Sequence
  TGF-beta -binding region - Protein 130
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTGGGGSGGGGSGGGGSGGGGSGGGG

SEQ ID NO: 44 - protein, 276 aa, Artificial Sequence
  TGF-beta -binding region - Protein 131
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDVQKSVNNDMIVTDNNGAVKF

SEQ ID NO: 45 - protein, 276 aa, Artificial Sequence
  TGF-beta -binding region - Protein 132
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDVQKSVNNDMIVTDNNGAVKF

SEQ ID NO: 46 - protein, 276 aa, Artificial Sequence
  TGF-beta -binding region - Protein 133
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTIPPHVQKSVNNDMIVTDNNGAVKF

SEQ ID NO: 47 - protein, 262 aa, Artificial Sequence
  TGF-beta -binding region - Protein 134
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFGGGGSGGGGSGGGGSG

SEQ ID NO: 48 - protein, 262 aa, Artificial Sequence
  TGF-beta -binding region - Protein 135
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFGGGGSGGGGSGGGGSGGGPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDNGAVKF

SEQ ID NO: 49 - protein, 224 aa, Artificial Sequence
  hIgG1Fc(CC) region
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 50 - protein, 224 aa, Artificial Sequence
  hIgG4Fc region
GPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 51 - protein, 217 aa, Artificial Sequence
  hIgG1Fc
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 216 aa, Artificial Sequence
  hIgG1Fc variant
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 53 - protein, 220 aa, Artificial Sequence
  hIgG1Fc variant
PPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 54 - protein, 226 aa, Artificial Sequence
  hIgG1Fc variant
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 55 - protein, 231 aa, Artificial Sequence
  hIgG1Fc variant
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 56 - protein, 231 aa, Artificial Sequence
  hIgG1Fc variant
EPKSSDKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 57 - protein, 216 aa, Artificial Sequence
  hIgG2Fc
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 58 - protein, 223 aa, Artificial Sequence
  hIgG2Fc variant
VECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 59 - protein, 228 aa, Artificial Sequence
  hIgG2Fc variant
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 60 - protein, 227 aa, Artificial Sequence
  hIgG2Fc variant
ERKSSVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPG

SEQ ID NO: 61 - protein, 227 aa, Artificial Sequence
  hIgG2Fc variant
ERKSSVESPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPG

SEQ ID NO: 62 - protein, 227 aa, Artificial Sequence
  hIgG2Fc variant
ERKSSVESPPSPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPG

SEQ ID NO: 63 - protein, 215 aa, Artificial Sequence
  hIgG2Fc variant
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPG

SEQ ID NO: 64 - protein, 219 aa, Artificial Sequence
  hIgG2Fc variant
PPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPG

SEQ ID NO: 65 - protein, 222 aa, Artificial Sequence
  hIgG2Fc variant
VECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPG

SEQ ID NO: 66 - protein, 217 aa, Artificial Sequence
  hIgG3Fc
APELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 67 - protein, 216 aa, Artificial Sequence
  hIgG3Fc variant
APELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPG

SEQ ID NO: 68 - protein, 220 aa, Artificial Sequence
  hIgG3Fc variant
PRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPG

SEQ ID NO: 69 - protein, 226 aa, Artificial Sequence
  hIgG3Fc variant
DTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPG

SEQ ID NO: 70 - protein, 231 aa, Artificial Sequence
  hIgG3Fc variant
EPKSSDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPG

SEQ ID NO: 71 - protein, 231 aa, Artificial Sequence
  hIgG3Fc variant
EPKSSDTPPPSPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPG

SEQ ID NO: 72 - protein, 231 aa, Artificial Sequence
  hIgG3Fc variant
EPKSSDTPPPSPRSPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPG

SEQ ID NO: 73 - protein, 217 aa, Artificial Sequence
  hIgG4Fc
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 74 - protein, 216 aa, Artificial Sequence
  hIgG4Fc variant
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 75 - protein, 220 aa, Artificial Sequence
  hIgG4Fc variant
PSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 76 - protein, 228 aa, Artificial Sequence
  hIgG4Fc variant
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 77 - protein, 228 aa, Artificial Sequence
  hIgG4Fc variant
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 78 - protein, 228 aa, Artificial Sequence
  hIgG4Fc variant
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLG

SEQ ID NO: 79 - protein, 228 aa, Artificial Sequence
  hIgG4Fc variant
ESKYGPPSPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 80 - protein, 228 aa, Artificial Sequence
  hIgG4Fc variant
ESKYGPPSPSSPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 81 - protein, 486 aa, Artificial Sequence
  Protein 61
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDNGAVKFGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 82 - protein, 492 aa, Artificial Sequence
  Protein 93
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDNGAVKFGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 83 - protein, 500 aa, Artificial Sequence
  Protein 94
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDNDMIVTDNNGAVKFGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 84 - protein, 486 aa, Artificial Sequence
  Protein 96
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDNGAVKFTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 85 - protein, 486 aa, Artificial Sequence
  Protein 97
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 86 - protein, 512 aa, Artificial Sequence
  Protein 99
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTIPPHVQKSVNNDMIVTDNNGAVKFGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 87 - protein, 500 aa, Artificial Sequence
  Protein 101
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTIPPHVQKSVNNDMIVTDNNGAVKFTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 88 - protein, 504 aa, Artificial Sequence
  Protein 106
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYPHVQKSVNNDMIVTDNNGAVKFTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 89 - protein, 480 aa, Artificial Sequence
  Protein 107
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 90 - protein, 484 aa, Artificial Sequence
  Protein 108
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDNGAVKFTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 91 - protein, 488 aa, Artificial Sequence
  Protein 109
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDNGAVKFTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 92 - protein, 486 aa, Artificial Sequence
  Protein 112
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYMIVTDNNGAVKFTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 93 - protein, 516 aa, Artificial Sequence
  Protein 113
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDPHVQKSVNNDMIVTDNNGAVKFTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 94 - protein, 516 aa, Artificial Sequence
  Protein 115
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDPHVQKSVNNDMIVTDNNGAVKFGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 95 - protein, 518 aa, Artificial Sequence
  Protein 116
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDIPPHVQKSVNNDMIVTDNNGAVKFTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 96 - protein, 500 aa, Artificial Sequence
  Protein 128
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTIPPHVQKSVNNDMIVTDNNGAVKFGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 97 - protein, 500 aa, Artificial Sequence
  Protein 129
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFGGGGSGGGGSGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 98 - protein, 500 aa, Artificial Sequence
  Protein 130
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTGGGGSGGGGSGGGGSGGGGSGGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 99 - protein, 500 aa, Artificial Sequence
  Protein 131
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDVQKSVNNDMIVTDNNGAVKFTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 100 - protein, 500 aa, Artificial Sequence
  Protein 132
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDVQKSVNNDMIVTDNNGAVKFTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 101 - protein, 500 aa, Artificial Sequence
  Protein 133
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTIPPHVQKSVNNDMIVTDNNGAVKFTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 102 - protein, 486 aa, Artificial Sequence
  Protein 134
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFGGGGSGGGGSGGGGSGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 103 - protein, 486 aa, Artificial Sequence
  Protein 135
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFGGGGSGGGGSGGGGSGGGPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDNGAVKFTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 104 - protein, 19 aa, Artificial Sequence
  Signal peptide
MDWTWRILFLVAAATGTHA

SEQ ID NO: 105 - protein, 486 aa, Artificial Sequence
  Protein 70
IPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 106 - DNA, 1461 bp, Artificial Sequence
  cDNA encoding Protein 61
attcctccacacgtgcagaaatccgtgaacaacgacatgatcgtgaccgacaacaatggcgccgtgaagttccctcagctgtgcaagttctgcgacgtgcggttctctacctgcgacaaccagaaatcctgcatgtccaactgctccatcacctccatctgcgagaagccccaagaagtgtgcgtcgccgtctggcggaagaacgacgagaacatcaccctggaaaccgtgtgccacgatcctaagctgccctaccacgacttcatcctggaagatgccgcctctcctaagtgcatcatgaaggaaaagaagaagcccggcgagactttcttcatgtgcagctgctcctccgacgagtgcaacgacaacatcatcttctccgaagagtacaacaccagcaatcccgacgataacaacggggctgtgaaattcccacagctctgtaaattttgtgatgtgcggttcagcacctgtgacaatcaaaagtcttgcatgagcaactgcagcatcaccagcatctgtgaaaaacctcaagaagtctgcgttgcagtttggcgcaaaaatgatgagaatatcacactcgagacagtgtgtcatgaccccaagctgccttatcatgatttcatcttggaggacgctgctagccccaagtgtattatgaaggaaaaaaagaaacctggggaaaccttctttatgtgctcctgcagcagcgacgagtgtaatgataacattattttcagtgaagagtataatacctccaatcctgacaacggcgctgtcaagtttgggcccccttgtcctccttgtccagctcctgaatttctcggcggaccctccgtgttcctgtttcctccaaagcctaaggacaccctgatgatctctcggacccctgaagtgacctgcgtggtggtggatgtgtcccaagaggaccctgaggtgcagttcaattggtacgtggacggcgtggaagtgcacaacgccaagaccaagcctagagaggaacagttcaactccacctacagagtggtgtccgtgctgaccgtgctgcaccaggattggctgaacggcaaagaatacaagtgcaaggtgtccaacaagggcctgccttccagcatcgaaaagaccatctccaaggccaagggccagcctagggaaccccaggtgtacacactgcctccaagccaagaggaaatgaccaagaaccaggtgtccctgacctgcctggtcaagggcttctacccttccgatatcgccgtggaatgggagagcaatggccagcctgagaacaactacaagaccacacctcctgtgctggactccgacggctccttctttctgtactcccgcctgaccgtggacaagtccagatggcaagagggcaacgtgttctcctgctccgtgatgcacgaggccctgcacaatcactacacccagaagtccctgtctctaagcttaggctga

SEQ ID NO: 107 - DNA, 1461 bp, Artificial Sequence
  cDNA encoding Protein 96
atcccccctcatgtgcagaagtccgtgaacaatgacatgatcgtgaccgataacaatggcgccgtgaagtttccacagctgtgcaagttctgtgacgtgaggtttagcacctgcgataatcagaagtcctgcatgagcaactgttctatcacatccatctgcgagaagccacaggaggtgtgcgtggccgtgtggcggaagaacgacgagaatatcaccctggagacagtgtgccacgatccaaagctgccctaccatgacttcatcctggaggatgctgcctctcctaagtgtatcatgaaggagaagaagaagccaggcgagaccttctttatgtgctcctgttccagcgacgagtgcaacgataatatcatcttcagcgaggagtataacacatctaatcccgacgataacaatggcgctgtgaagtttcctcagctgtgcaaattttgtgacgtgagattttctacctgtgataatcagaagagctgcatgtctaactgttccatcacatctatttgtgaaaaacctcaggaagtgtgcgtggccgtgtggagaaaaaatgatgaaaacatcaccctggaaacagtgtgccacgatcctaagctgccatatcacgacttcatcctggaagacgctgccagcccaaaatgcattatgaaagagaagaagaagcccggtgagaccttctttatgtgcagctgttcttctgatgaatgtaatgacaatatcatcttctccgaggagtataacacaagcaatcctgacaacggcgctgtgaagtttacccacacatgcccaccatgtcctgctccagagctgctgggaggaccatccgtgttcctgtttcctccaaagcccaaggacaccctgatgatcagcaggacccccgaggtgacatgcgtggtggtggacgtgtctcacgaggaccccgaggtgaagtttaattggtacgtggatggcgtggaggtgcataacgctaagaccaagccaagggaggagcagtacaactctacctatcgggtggtgtccgtgctgacagtgctgcatcaggattggctgaatggcaaggagtataagtgcaaggtgtccaacaaggctctgcccgcccctatcgagaagaccatcagcaaggctaagggccagcccagagagcctcaggtgtacacactgcccccttctcgcgacgagctgaccaagaatcaggtgtccctgacatgtctggtgaagggcttctatccttccgatatcgctgtggagtgggagagcaacggacagccagagaacaattacaagaccacaccacccgtgctggactctgatggctccttctttctgtatagcaagctgaccgtggacaagtctaggtggcagcagggcaacgtgttctcctgttctgtgatgcacgaggccctgcacaaccattacacacagaagtccctgagcctgtctcctggctga

SEQ ID NO: 108 - DNA, 1503 bp, Artificial Sequence
  cDNA encoding Protein 101
atcccccctcatgtgcagaagtccgtgaacaatgacatgatcgtgacagataacaatggcgccgtgaagtttccccagctgtgcaagttctgtgacgtgaggtttagcacctgcgataaccagaagtcctgcatgagcaattgttctatcacatccatctgcgagaagcctcaggaggtgtgcgtggccgtgtggcggaagaacgacgagaatatcaccctggagacagtgtgccacgatcccaagctgccttaccatgacttcatcctggaggatgctgcctctccaaagtgtatcatgaaggagaagaagaagcccggcgagaccttctttatgtgctcctgttccagcgacgagtgcaacgataatatcatcttcagcgaggagtataacgatatgattgtcacagataacaatggcgctgtgaagtttccacagctgtgcaaattttgtgacgtgagattttccacctgtgataaccagaagagctgcatgtctaattgttccatcacaagcatctgcgagaagccacaggaagtgtgcgtggccgtgtggagaaaaaatgatgaaaacatcaccctggaaacagtgtgccacgatccaaagctgccctatcacgacttcatcctggaagacgctgccagccctaaatgcattatgaaagagaagaagaagccaggtgagaccttctttatgtgcagctgttcttctgatgaatgtaatgacaatatcatcttctccgaggagtataacaccatccctccacacgtgcagaagtctgtgaataacgatatgattgtgaccgacaataacggtgctgtgaagtttacccatacatgcccaccttgtccagctccagagctgctgggaggaccatccgtgttcctgtttccacccaagcctaaggacaccctgatgatcagcaggaccccagaggtgacatgcgtggtggtggacgtgtctcacgaggaccccgaggtgaagtttaactggtacgtggatggcgtggaggtgcataatgctaagaccaagcctagggaggagcagtacaactctacctatcgggtggtgtccgtgctgacagtgctgcaccaggactggctgaacggcaaggagtataagtgcaaggtgtccaataaggctctgcctgccccaattgagaagaccatcagcaaggctaagggccagcctagagagccacaggtgtacacactgcctccatctcgcgacgagctgaccaagaaccaggtgtccctgacatgtctggtgaagggcttctatccctccgatatcgctgtggagtgggagagcaacggacagcctgagaacaattacaagaccacaccccctgtgctggactctgatggctccttctttctgtatagcaagctgaccgtggataagtctaggtggcagcagggcaacgtgtttagctgttctgtgatgcatgaggccctgcacaatcattacacacagaagtccctgagcctgtctccaggctga

SEQ ID NO: 109 - DNA, 1503 bp, Artificial Sequence
  cDNA encoding Protein 128
atcccccctcatgtgcagaagagcgtcaataacgacatgatcgtgaccgataacaatggcgccgtgaagttcccacagctgtgcaagttctgtgacgtgaggtttagcacctgcgataatcagaagtcttgcatgtccaactgtagcatcacatctatctgcgagaagccacaggaggtgtgcgtggccgtgtggcggaagaacgacgagaatatcaccctggagacagtgtgccacgatccaaagctgccctaccatgactttatcctggaggatgccgctagccctaagtgtatcatgaaggagaagaagaagccaggcgagacattctttatgtgctcttgttccagcgacgagtgcaacgataatatcatcttctccgaggagtataacgatatgatcgtgacagacaataacggcgccgtgaagtttcctcagctgtgcaaattttgtgacgtgagattttctacctgtgataatcagaagtcctgcatgagcaactgttctatcacatccatttgtgaaaaacctcaggaagtgtgcgtggccgtgtggagaaagaatgacgagaacatcaccctggagacagtgtgccatgatcctaagctgccataccatgacttcatcctggaagacgccgcttcccccaaatgcattatgaaagagaagaagaagcctggcgagacattcttcatgtgcagctgttcttctgatgagtgcaacgataacatcatcttttctgaggagtacaacaccatcccccctcacgtgcagaagtctgtgaacaatgacatgatcgtgacagataacaatggagctgtgaagttcgggcccccatgccctccatgtccagctcctgagtttctgggcggcccatccgtgttcctgtttccccctaagcccaaggacaccctgatgatctccaggacccctgaggtgacatgcgtggtggtggacgtgagccaggaggaccccgaggtgcagttcaattggtacgtggatggcgtggaggtgcacaacgccaagacaaagcctagagaggagcagtttaatagcacctaccgcgtggtgtctgtgctgacagtgctgcatcaggattggctgaatggcaaggagtataagtgcaaggtgtctaacaagggcctgccaagctctatcgagaagaccatctccaaggctaagggacagccaagggagcctcaggtgtacacactgccacccagccaggaggagatgaccaagaatcaggtgtctctgacatgtctggtgaagggcttctatccatctgacatcgctgtggagtgggagtccaacggccagcccgagaacaattacaagaccacacctccagtgctggacagcgatggctctttctttctgtattccaggctgaccgtggataagagccggtggcaggagggcaacgtgttttcctgtagcgtgatgcacgaagcactgcacaaccactacactcagaaatcactgtcactgtccctgggctaa

SEQ ID NO: 110 - DNA, 63 bp, Artificial Sequence
  DNA encoding Kozak sequence and signal peptide
gccaccatggactggacctggagaatcctgttcctggtggctgctgctaccggaacacacgct

SEQ ID NO: 111 - DNA, 63 bp, Artificial Sequence
  DNA encoding Kozak sequence and signal peptide
gccaccatggactggacttggagaatcctgttcctggtcgccgccgcaactggtactcatgct